(19) United States
(12) Patent Application Publication
(10) Pub. No.: US 2018/0161272 A1
(43) Pub. Date: Jun. 14, 2018

(54) LIPOSOMAL NANOCONSTRUCTS AND METHODS OF MAKING AND USING THE SAME

(71) Applicant: The General Hospital Corporation, Boston, MA (US)

(72) Inventors: Girgis Obaid, Boston, MA (US); Tayyaba Hasan, Arlington, MA (US)

(21) Appl. No.: 15/576,583

(22) PCT Filed: May 26, 2016
(86) PCT No.: PCT/US16/34319

§ 371 Date: Nov. 22, 2017

Related U.S. Application Data
(60) Provisional application No. 62/166,353, filed on May 26, 2015.

(51) Int. Cl. A61K 9/127; A61K 47/54; A61K 47/69; A61K 41/00; A61P 35/00

(52) U.S. Cl. CPC A61K 9/127; A61K 47/544; A61P 35/00; A61K 41/0071; A61K 47/6913

(57) ABSTRACT

Provided herein is a liposome comprising: a) a conjugate comprising a lysophospholipid and a photosensitizer; b) a first derivatized phospholipid comprising a first phospholipid and a strained cyclooctyne moiety; c) a second derivatized phospholipid comprising a second phospholipid and a polyethylene glycol polymer; and d) a cationic or anionic lipid. Also provided herein is a liposome comprising: a) a conjugate comprising a lysophospholipid and a photosensitizer; b) a first derivatized phospholipid comprising a first phospholipid and a targeting moiety; c) a second derivatized phospholipid comprising a second phospholipid and a polyethylene glycol polymer; and d) a cationic or anionic lipid. The liposomes provided herein can be used, for example, in the treatment of cancer or in the imaging of cancer tumors.

### CLAIM OF PRIORITY

This application claims the benefit of U.S. Provisional Application Ser. No. 62/166,353, filed on May 26, 2015, which is incorporated herein by reference in its entirety.

### TECHNICAL FIELD

This disclosure relates to liposomes comprising a conjugated photosensitizer.

### BACKGROUND

Nanoscale drug delivery systems (e.g., liposomes) allow the release of nutrients and drugs selectively to molecular targets. Liposomes are often composed of a phospholipid-containing outer layer (containing, for example, phosphatidylcholine and/or egg phosphatidylethanolamine) and an aqueous core. Examples of types of liposomes include multilamellar vesicles (which include several lamellar phase lipid bilayers), small unilamellar vesicles (which includes one lipid bilayer), large unilamellar vesicles, and cochleate vesicles (see, e.g, WO/09032716). Due to the presence of both hydrophobic and hydrophilic regions, liposomes can be loaded with hydrophobic and/or hydrophilic molecules, including for example, drugs, DNA, and/or other bioactive molecules. The surface of liposomes can be accessorized with ligands to bind to specific targets, thus enabling selective delivery of molecules loaded in the liposome to the targets. Liposomes release molecules by a variety of means, including, for example, fusion with the bilayer of a cell membrane (see, e.g., Advanced Drug Delivery Reviews 38(3):207-232, 1993) or macrophage phagocytosis.

### SUMMARY

Provided herein is a liposome comprising: a) a conjugate comprising a lysophospholipid and a photosensitizer; b) a first derivatized phospholipid comprising a first phospholipid and a strained cyclooctyne moiety; c) a second derivatized phospholipid comprising a second phospholipid and a polyethylene glycol polymer; and d) a cationic or anionic lipid. Also provided herein is a liposome comprising: a) a conjugate comprising a lysophospholipid and a photosensitizer; b) a first derivatized phospholipid comprising a first phospholipid and a targeting moiety; c) a second derivatized phospholipid comprising a second phospholipid and a polyethylene glycol polymer; and d) a cationic or anionic lipid. The liposomes provided herein can be used, for example, in the treatment of cancer or in the imaging of cancer tumors.

Further provided herein are methods of treating a cancer in a patient, the method comprising administering to the patient a therapeutically effective amount of a liposome provided herein; and irradiating the patient to break the liposome.

The liposomes provided herein can also be used to image a cancer in a patient. In some embodiments, the methods include administering to the patient an effective amount of a liposome provided herein; irradiating the patient to break the liposome; and imaging the patient.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 depicts median BPD fluorescence intensity vs. concentration of BPD in the liposome formulation in OVCAR-5 cells for (1) liposomes with BPD embedded in the lipid bilayer, (2) liposomes with BPD conjugated to 16:0 Lyso PC-BPD, and (3) liposomes with BPD conjugated to 20:0 Lyso PC-BPD.

FIG. 2 depicts Z-average diameters and polydispersity indices for liposomes containing 16:0 Lyso PC-BPD, sterically stabilized by the incorporation of 5%, 4%, 3%, 2%, 1% and 0.5% of the phospholipid 1,2-dioleoyl-sn-glycero-3-phosphoethanolamine-N-[methoxy(polyethylene glycol)-2000] (DSPE-mPEG₂₀₀₀).

FIGS. 3A-3C show ζ-potential, Z average diameter, and polydispersity indices (PDI), respectively, for ADIBO-modified, 16:0 Lyso PC-BPD-containing liposomes containing DOTAP, DOPG, or no added charge.

FIG. 4 shows the quantitation of cellular update (pmol 16:0 Lyso PC-BPD/μg cell protein) for non-targeted DOTAP- and DOPG-containing liposomes in OVCAR-5 cells, A431 cells, and T47D cells.

FIG. 5 shows the fold release of liposomes with (1) Lyso PC-BPD (uppermost plot), (2) Lyso PC-BPD and azide (middle plot), and (3) no Lyso PC-PBD (lowest plot) vs. fluence (light dose) with 690 nm light.

FIG. 6A is a schematic representation of a liposome surface bound to cetuximab-protein Z through copper-free click chemistry. Protein Z is site-specifically bound to the Fc region of any IgG molecule (e.g., cetuximab) and is photocross-linked through an unnatural benzoylphenylalanine amino acid. The terminal azide on the peptide-bound protein Z is click conjugated to the optimal surface molar ratio of DSPE-PEG₂₀₀₀-ADIBO, a strained-cyclooctyne derivatized phospholipid. FIG. 6B is a schematic representation of a liposome surface bound to azido-cetuximab Z through copper-free click chemistry. The terminal azide on the PEG4 molecules stochastically attached to the targeting protein (e.g., cetuximab) is click conjugated to the optimal molar ratio of DSPE-PEG₂₀₀₀-ADIBO, a strained-cyclooctyne derivatized phospholipid.

FIGS. 7A-B show the UV-visible spectrum of the site-specific azide conjugate and a structural schematic, respectively. Stochastic introduction of azide moieties to cetuximab was achieved using an N-hydroxysuccinimidyl ester of PEG4-azide. A single fluorophore (5-FAM for site-specific cetuximab and AlexaFluor® 488 for stochastic cetuximab) was introduced into each antibody conjugate. The ratio of protein Z to cetuximab is 1.13±0.17.

FIGS. 8A-B show the UV-visible spectrum of the stochastic azide conjugate incorporating AlexaFluor® 488 and its structural schematic, respectively. The ratio of AlexaFluor® 488 to Cetuximab is 1.00±0.04.

FIG. 9 shows comparisons of liposome size (uppermost plots in each figure) and polydispersity index (lower plots in each figure) for site-specific (FIG. 9A) and stochastic (FIG. 9B) conjugation.

FIG. 10 depicts a transmission electron microscope (TEM) image of liposomes site-specifically click conjugated to cetuximab-Protein Z imaged at an accelerating voltage of 80 kV and a magnification of 46,000×.

FIG. 11 shows comparisons of ζ-potential vs. conjugation efficiency for site-specific (uppermost plot, at left) and stochastic (lower plot, at left) conjugation.

FIG. 12 shows comparisons of the number of cetuximab conjugated per liposome vs. conjugation efficiency for site-specific (uppermost plot) and stochastic (lower plot) conjugation.

FIG. 13 shows comparisons of binding selectivity in A431 cells (high EGFR; uppermost plot), T47D cells (low EGFR; middle plot) and CHO-WT cells (no EGFR; lower plot).

FIG. 14 shows comparisons of binding selectivity of a liposome-cetuximab conjugate with AlexaFluor® 488 in A431 cells (high EGFR; uppermost plot), T47D cells (low EGFR; middle plot) and CHO-WT cells (no EGFR; lower plot).

FIG. 15 shows flow cytometry data for targeted and non-specific liposomes, as well as targeted and non-specific liposomes when EGFR was blocked.

FIG. 16 shows the cell viabilities following selective photodynamic therapy using 16:0 Lyso PC-BPD liposomes conjugated site-specifically and stochastically to cetuximab.

FIG. 17 demonstrates that stochastic azido modification of Trastuzumab (anti-HER-2 antibody, Herceptin®) and transferrin (natural ligand of transferrin receptor) exhibit cellular selectivity of binding when clicked to ADIBO-modified, 16:0 Lyso PC-BPD-containing liposomes.

FIG. 18A shows ADIBO-modified liposomes with no membrane entrapped 16:0 Lyso PC-BPD were loaded with the water-soluble photosensitizer chlorin e6 monoethylene diamine monoamide (CMA) and shown to offer cellular selectivity when stochastically conjugated to cetuximab. A schematic of the liposome is shown in FIG. 18B.

FIG. 19 shows confocal fluorescence microscopy images of non-targeted and stochastically targeted liposomes in OVCAR-5 cells at progressive time intervals, indicating cellular internalization of the targeted liposomes.

FIG. 20 shows 0.2% DSPE-PEG₂₀₀₀-NH₂ was incorporated within ADIBO-modified liposomes and conjugated with amine-reactive NHS-esters of the near-infrared dyes to prepare a panel of stable click-reactive liposomes capable of use in in vivo imaging.

FIG. 21A shows size and polydispersity indices of liposomes that include the various dyes, and FIG. 21B shows UV-visible spectra of each.

FIG. 22 shows median Lissamine Rhodamine B intensity as a function of the Cetuximab-Protein Z (Cet-Pz) density per liposome, demonstrating that the fluorescently labeled liposomes selectively bind to A431 cells (high EGFR; upper plot) compared to T47D cells (low EGFR; lower plot).

FIG. 23 shows the average corrected fluorescence of IRDye® 680RD (upper plot) and IRDye® 800CW (lower plot) vs. time, demonstrating selective binding of the IRDye680RD labeled liposome click conjugated to Cetuximab (Erbitux) to subcutaneous U251 tumors, compared to co-injected IRDye800CW labeled liposome click conjugated to a sham human IgG1 control.

FIG. 24 shows that the targeted liposome (right bar in each pair) was more selective for the A431 tumor than the non-targeted liposome (left bar in each pair of bars).

FIG. 25 shows that the fluorescence of 16:0 Lyso PC-BPD can be used to quantitatively image the biodistribution and confirm the tumor selectivity of stochastically click conjugated liposomes in mice with subcutaneous A431 (high EGFR) tumors 24 hours after administration of 0.25 mg/kg BPD equivalent, compared to non-conjugated DIBO-containing 16:0 Lyso PC-BPD liposome controls.

FIG. 26 depicts a structural schematic of a liposome including the 16:0 Lyso PC-BPD photosensitizer with PEG chains shown.

FIG. 27 is a conceptual representation of a targeted liposome containing agents within the bilayer or within a core-entrapped PEG-PLGA nanoparticle.

FIG. 28 provides flow cytometry data and shows the median photosensitizer (BPD) intensity from liposomes reacted with v0, 10, 50 or 100 Cetuximab-Protein Z conjugates (Cet-Pz) per liposome.

FIG. 29A provides flow cytometry data depicting the median photosensitizer (BPD) intensity originating from the liposome membrane. FIG. 29B shows the median 5-FAM fluorescence intensity originating from the fluorescently labeled peptide attached to Protein Z.

FIGS. 30A-30B show a correlation between the median fluorescence intensity of BPD and the 5-FAM Protein Z bound to Cetuximab in A431 cells is depicted in FIG. 30A (high EGFR) and in T47D cells in FIG. 30B (low EGFR). The high correlation coefficient is indicative of the integrity of the Cetuximab click conjugated to the liposome.

FIGS. 31A-C depict flow cytometry data, where the median BPD intensity is the average of 50,000 individually measured cells. The cells were incubated for 30 min, 90 min or 180 minutes with liposomes click conjugated to 50 Cetuximab/liposome (FIG. 31A) versus non-conjugated liposomes (FIG. 31B). Selectivity of the liposomes (FIG. 31C) is representative of the median BPD intensity of cells incubated with targeted liposomes divided by the median BPD intensity of cells incubated with non-specific liposomes.

FIG. 32 depicts the results of an MTT assay of A431 and OVCAR-5 cells (high EGFR) and T47D cells (low EGFR) following PDT treatment with 20 J/cm² of 690 nm laser light. The stochastically targeted liposomes were incubated for 24 hours with the cells.

FIG. 33 shows median 5-FAM fluorescence intensity as a function of Cetuximab-Protein Z density per liposome, also demonstrating high selectivity for A431 cells (upper plot) compared to T47D cells (lower plot). Optimal density occurs at about 100 Cet-Pz/liposome.

FIG. 34 shows optimal Cetuximab-Protein Z density for A431 cells at 500 nM and 100 nM concentrations of Lissamine Rhodamine B (uppermost plot and next plot down, respectively) and for T47D cells at 500 nM and 100 nM concentrations of Lissamine Rhodamine B (lower two plots).

FIG. 35 shows fold selectivity for a variety of cancer cell lines using GOA_111-CetPz (10 CetPz/liposome) median Lissamine Rhodamine B intensity, at 500 nM Lissamine Rhodamine B equivalent. 100 μL liposome samples were incubated for 30 minutes with 50,000 cells at 37° C.

FIG. 36 shows the transmission electron microscope (TEM) image of a liposome processed according to the foregoing procedure.

FIG. 37 shows fold selectivity vs. nM equivalent of Rhodamine, showing that liposomes that are click conjugated to Cetuximab and include both a lipid anchored Rhodamine dye and hydrophobically entrapped free BPD show differential levels of selectivity compared to liposomes without antibody conjugation.

FIG. 38 shows confocal fluorescence microscope images of MGG6 cell neurospheres (nuclei stained with Hoechst 33324, blue) incubated with Rhodamine labelled liposomes (red) for 30 minutes. FIG. 38 shows that at 30 minutes, targeted liposomes that are click conjugated to Cetuximab-anti EGFR show preferential binding (FIG. 38A), compared to non-targeted liposomes (FIG. 38B).

FIG. 39A provides an example formulation incorporating a lipid anchored fluorophore Lissamine Rhodamine B-DPPE. Size and polydispersity indices for three samples (50% v/v GOA_111+5×ADIBO 1 (uppermost plot), 50% v/v GOA_111+5×ADIBO 2 (middle plot), and 50% v/v GOA_111+5×ADIBO 3 (lowest plot) are shown in FIG. 39B.

FIG. 40 shows size and polydispersity indices for each sample.

### DETAILED DESCRIPTION

Provided herein is a stable copper-free click chemistry liposome. The liposome has been chemically optimized to stably incorporate a lipid-anchored photosensitizer molecule that can act as a photodynamic therapy agent, a fluorophore, and/or a mediator for the spatiotemporally-controlled phototriggered release of a liposomal cargo. The liposomal cargo could include secondary biological or chemical therapeutics, free or nanoparticle-bound, that can synergize with the photosensitizer. The liposome provided herein is chemically tuned for stability and selectivity of binding and phototoxicity. For example, the liposome can include a targeting moiety such as an antibody to selectively target the photosensitizer and the optional cargo. The liposome can be further tuned for the stable modular and non-interfering conjugation of near-infrared imaging agents to allow for the paralleled deep-tissue tracing and imaging of the targeted liposome.

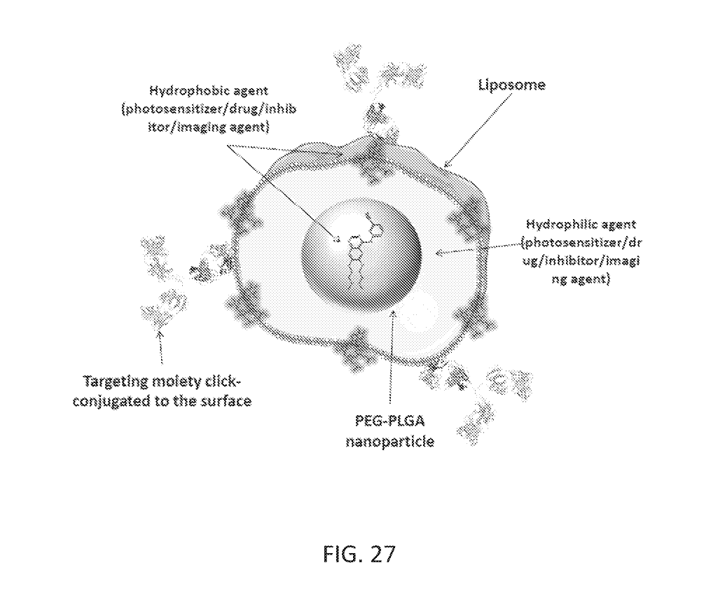

For example, FIG. 27 is a conceptual representation of a targeted liposome as provided herein. In some embodiments, the liposome contains agents within the bilayer or within a core-entrapped PEG-PLGA nanoparticle. These agents can include photosensitizers, imaging agents, drugs, or kinase inhibitors. Hydrophilic photosensitizers, imaging agents, drugs, or kinase inhibitors can be encapsulated within the aqueous core. The surface of the liposome is conjugated to a targeting moiety, such as an antibody, through copper-free click chemistry using, for example, a liposomal composition of DSPE-PEG₂₀₀₀-ADIBO.

Provided herein is a liposome comprising: a) a conjugate comprising a lysophospholipid and a photosensitizer; b) a first derivatized phospholipid comprising a first phospholipid and a strained cyclooctyne moiety; c) a second derivatized phospholipid comprising a second phospholipid and a polyethylene glycol polymer; and d) a cationic or anionic lipid.

Also provided herein is a liposome comprising: a) a conjugate comprising a lysophospholipid and a photosensitizer; b) a first derivatized phospholipid comprising a first phospholipid and a targeting moiety; c) a second derivatized phospholipid comprising a second phospholipid and a polyethylene glycol polymer; and d) a cationic or anionic lipid.

A photosensitizer, as used herein, refers to a small molecule photodynamic therapy agent, a fluorophore, and/or a mediator for the spatiotemporally-controlled phototriggered break the liposome and/or release a liposomal cargo. In some embodiments, the photosensitizer is hydrophobic. In some embodiments, the photosensitizer is hydrophilic. In some embodiments, a photosensitizer is a porphyrin photosensitizer. For example, the photosensitizer comprises a benzoporphyrin moiety. In some embodiments, the photosensitizer is a benzoporphyrin derivative (BPD).

In some embodiments, the photosensitizer is selected from a class of photosensitizers including porphyrins, chlorins, bateriochlorins, phthalocyanines, naphthalocyanines, texaphrins, anthraquinones, anthracyclins, perylenequinones, xanthenes, cyanines, acridines, phenoxazines, phenothiazines, triarylmethanes, chalcogenapyrylium dyes, and phthalein dyes. In some embodiments, the photosensitizer is selected from verteporfin (3-[(23S,24R)-14-ethenyl-5-(3-methoxy-3-oxopropyl)-22,23-bis(methoxycarbonyl)-4,10,15,24-tetramethyl-25,26,27,28-tetraazahexacyclo[16.6.1.13,6.18,11.113,16.019,24]octacosa-1,3,5,7,9,11(27),12,14,16,18(25),19,21-dodecaen-9-yl]propanoic acid); protoporphyrin IX; temoporfin (3,3′,3″,3′″-(2,3-dihydroporphyrin-5,10,15,20-tetrayl)tetraphenol); motoxafin lutetium; 9-Acetoxy-2,7,12,17-tetrakis-(β-methoxyethyl)-porphycene (ATMPn); chlorin e6; chlorin monoethylene diamine monoamide; protoporphyrin IX; zinc phthalocyanine; silicon phthalocyanine Pc 4; and naphthalocyanines.

In some embodiments, the photosensitizer is conjugated to a lysophospholipid. See, for example, J. Lovell, C. Jin, E. Huynh, H. Jin, C. Kim, J. Rubinstein, W. Chan, W. Cao, L. Wang and G. Zheng. Porphysome nanovesicles generated by porphyrin bilayers for use as multimodal biophotonic contrast agents 2011 Nature Materials, 10, 324-332. In some embodiments, the photosensitizer is aqueously encapsulated. In some embodiments, the photosensitizer is conjugated to a second derivatized phospholipid as described herein (e.g., through conjugation to the polyethylene glycol polymer). In some embodiments, a photosensitizer is conjugated to a phospholipid as provided herein through reaction with the phosphate head group of the phospholipid (see K. A. Riske, T. P. Sudbrack, N. L. Archilha, A. F. Uchoa, A. P. Schroder, C. M. Marques, M. S. Baptista, R. Itri, Biophys. J. 97 (2009) 1362-1370). In some embodiments, a photosensitizers is conjugated to the acyl chains of the phospholipid (see T. Komatsu, M. Moritake, A. Nakagawa, E. Tsuchida, Chemistry 8 (2002) 5469-5480). In some embodiments, a photosensitizer is conjugated to another component of the liposome such as a cholesterol.

As used herein, a lysophospholipid is any derivative of a phospholipid in which one or both acyl derivatives have been removed by hydrolysis. In some embodiments, a lysophospholipid includes 14 to 22 carbons in the fatty acid chain. For example, the lysophospholipid can include 16 to 20 carbons in the fatty acid chain. In some embodiments, the lysophospholipid includes an unsaturated fatty acid chain. In some embodiments, the lysophospholipid is selected from 16:0 lysophospholipid, 20:0 lysophospholipid, and combinations thereof.

The conjugate comprising a lysophospholipid and a photosensitizer can be present in the liposome at about 0.01 mol percent to about 1 mole percent. For example, about 0.05 mol percent to about 0.5 mole percent in the liposome; about 0.08 mol percent to about 0.12 mole percent in the liposome. In some embodiments, the conjugate comprising a lysophospholipid and a photosensitizer is present at about 0.1 to about 0.2 mol percent in the liposome. For example, the conjugate comprising a lysophospholipid and a photosensitizer can be present at about 0.1 mol percent in the liposome.

In some embodiments, a conjugate (e.g., a BPD conjugate of 16:0 lysophospholipid (LysoPC) and/or 20:0 LysoPC and formed stable liposomes with the 16:0 LysoPC-BPD and 20:0 LysoPC-BPD) can be stably embedded into the phospholipid bilayer with the surface fully optimized for click conjugation of targeting ligands. Not all conjugations are as suitable as others. For example, conjugation of BPD's carboxylate to cholesterol's hydroxyl removes any polarity that either molecule exhibited, and no stable conformation can exist within the amphiphilic lipid bilayer and encapsulation is near 0%. Similarly, amide conjugation of hydrophobic BPD to the hydrophilic terminal of DSPE-PEG₂₀₀₀-amine anchors BPD into the membrane but yields liposomes that are unstable due to the peripheral hydrophobic stacking of inter-liposomal surface BPD moieties. A shown herein, stable liposomes can be formed with conjugation of a photosensitizer to a lysophospholipid (e.g., the 16:0 LysoPC-BPD and 20:0 LysoPC-BPD). The liposomes provided herein block non-specific leakage of the photosensitizers to surrounding cells like that observed with hydrophobic BPD entrapment in a lipid bilayer.

Conjugation of the lysophospholipid with a hydrophobic photosensitizer containing chemically reactive functional groups such —OH, —COOH, —NH₂, and —SH can be achieved using standard conjugation methods to lysophospholipids such as those provided herein (e.g., lysophospholipids and lysophospholipid-linker derivatives (e.g., lysophospholipid-PEG derivatives). Conjugation of a hydrophillic photosensitizer containing chemically reactive functional groups such —OH, —COOH, —NH₂ and —SH can be achieved through conjugation to derivatives of the phosphate head groups of phospholipids such as DPPC, DPPE, DSPE-PEG-NH2, DSPE-PEG-COOH, DSPE-PEG-OH, DOPG or to the polar —OH group of cholesterol.

A first phospholipid as described herein can be selected from the group consisting of: a phosphatidylcholine; a phosphatidic acid; a phosphatidylethanolamime; a phosphatidylglycerol; and a phosphatidylserine. Non-limiting examples of a first phospholipid of the liposomes provided herein include hydrogenated soy phosphotidylcholine (HSPC); 1,2-didecanoyl-sn-glycero-3-phosphocholine (DDPC); 1,2-dierucoyl-sn-glycero-3-phosphate (DEPA); 1,2-dielaidoyl-sn-glycero-3-phosphocholine (DEPC); 1,2-dierucoyl-sn-glycero-3-phosphoethanolamine (DEPE); 1, 2-dielaidoyl-phosphatidylglycerol (DEPG); 1,2-dilinoleoyl-sn-glycero-3-phosphocholine (DLOPC); 1,2-dilauroyl-sn-glycero-3-phosphate (DLPA); 1,2-dilauroyl-sn-glycero-3-phosphocholine (DLPC); 1,2-dilauroyl-sn-glycero-3-phosphoethanolamine (DLPE); 1,2-dilauroyl-sn-glycero-3-phosphatidylglycerol (DLPG); 1,2-dilauroyl-sn-glycero-3-phosphoserine (DLPS); 1,2-dimyristoyl-sn-glycero-3-phosphate (DMPA); 1,2-dimyristoyl-sn-glycero-3-phosphocholine (DMPC); 1,2-dimyristoyl-sn-glycero-3-phosphoethanolamine (DMPE); 1,2-dimyristoyl-sn-glycero-3-phosphatidylglycerol (DMPG); 1,2-dimyristoyl-sn-glycero-3-phosphoserine (DMPS); 1,2-dioleoyl-sn-glycero-3-phosphate (DOPA); 1,2-dioleoyl-sn-glycero-3-phosphocholine (DOPC); 1,2-dioleoyl-sn-glycero-3-phosphoethanolamine (DOPE); 1,2-dioleoyl-sn-glycero-3-phosphatidylglycerol (DOPG); 1,2-dioleoyl-sn-glycero-3-phosphoserine (DOPS); 1,2-dipalmitoyl-sn-glycero-3-phosphate (DPPA); 1,2-dipalmitoyl-sn-glycero-3-phosphocholine (DPPC); 1,2-dipalmitoyl-sn-glycero-3-phosphoethanolamine (DPPE); 1,2-dipalmitoyl-sn-glycero-3-phosphatidylglycerol (DPPG); 1,2-dipalmitoyl-sn-glycero-3-phosphoserine (DPPS); 1,2-distearoyl-sn-glycero-3-phosphate (DSPA); 1,2-distearoyl-sn-glycero-3-phosphocholine (DSPC); 1,2-distearoyl-sn-glycero-3-phosphoethanolamine (DSPE); 1,2-distearoyl-sn-glycero-3-phosphatidylglycerol (DSPG); 1,2-distearoyl-sn-glycero-3-phosphoserine (DSPS); 1-myristoyl-2-palmitoyl-sn-glycero 3-phosphocholine (MPPC); 1-myristoyl-2-stearoyl-sn-glycero-3-phosphocholine (MSPC); 1-palmitoyl-2-myristoyl-sn-glycero-3-phosphocholine (PMPC); 1-palmitoyl-2-oleoyl-sn-glycero-3-phosphocholine (POPC); 1-palmitoyl-2-oleoyl-sn-glycero-3-phosphoethanolamine (POPE); 1-palmitoyl-2-oleoyl-sn-glycero-3-phosphatidylglycerol (POPG); 1-palmitoyl-2-stearoyl-sn-glycero-3-phosphocholine (PSPC); 1-stearoyl-2-myristoyl-sn-glycero-3-phosphocholine (SMPC); and 1-stearoyl-2-oleoyl-sn-glycero-3-phosphocholine (SOPC); 1-stearoyl-2-palmitoyl-sn-glycero-3-phosphocholine (SPPC), and combinations thereof. In some embodiments, the first phospholipid is 1,2-distearoyl-sn-glycero-3-phosphoethanolamine (DSPE).

In some embodiments, the first derivatized phospholipid further comprises a linker. For example, the linker can be a polyethylene glycol, wherein the polyethylene glycol is divalent and is a linker between the first phospholipid and the strained cyclooctene moiety or is a linker between the first phospholipid and the targeting moiety. In some embodiments, the linker can at as a linker between the first phospholipid and the copper-free click chemistry reaction product of the strained cyclooctyne and the targeting moiety.

In some embodiments, the polyethylene glycol has a molecular weight of about 350 g/mol to about 30,000 g/mol. For example, the polyethylene glycol can have a molecular weight selected from the group consisting of 350 g/mol, 550 g/mol, 750 g/mol, 1000 g/mol, 2000 g/mol, 3000 g/mol, 5000 g/mol, 10,000 g/mol, 20,000 g/mol, or 30,000 g/mol. In some embodiments, the polyethylene glycol has a molecular weight of 2000 g/mol.

In some embodiments, the first derivatized phospholipid comprises DSPE-PEG₂₀₀₀.

Non-limiting examples of a strained cyclooctyne include an aza-dibenzocyclooctyne (ADIBO), dibenzocyclooctyne (DIBO), (OCT), aryl-less octyne (ALO), monofluorinated cyclooctyne (MOFO), difluorinated cyclooctyne (DIFO), biarylazacyclooctynone (BARAC), or a dimethoxyazacyclooctyne (DIMAC) moiety. Prior to reaction with the first phospholipid or the linker, the strained cycloocytne can be selected from the group consisting of: dibenzocyclooctyne-N-hydroxysuccinimidyl ester (ADIBO-NHS), dibenzocyclooctyne-C6-N-hydroxysuccinimidyl ester, dibenzocyclooctyne-sulfo-N-hydroxysuccinimidyl ester, dibenzocyclooctyne-PEG(4, 5, 13 or n)-N-hydroxysuccinimidyl ester, dibenzocyclooctyne-S—S—N-hydroxysuccinimidyl ester, dibenzocyclooctyne-maleimide, dibenzocyclooctyne-PEG(4, 5, 13 or n)-maleimide, and (1R,8S,9s)-bicyclo[6.1.0]non-4-yn-9-ylmethyl N-succinimidyl carbonate.

A targeting moiety as used herein a targeting moiety can include one more of a folate, a RGD peptide, a natural protein ligand (such as TNF, EGF, transferrin), an affibody, an antibody, an antibody fragment, an engineered antibody-based protein, a cancer associated receptor, a folate re captor, a transferring receptor, a HER-2 receptor, avb5 inegrins, and somatostatin receptors. In some embodiments, the targeting moiety is an antibody.

In some embodiments, the targeting moiety comprises an azide moiety prior to conjugating with the first derivatized phospholipid. In some embodiments, the targeting moiety comprises a strained cyclooctyne as provided herein prior to conjugating with the first phospholipid. In some embodiments, the targeting moiety is conjugated to the first phospholipid using Cu-free click chemistry. For example, the azide or strained cyclooctyne on the targeting moiety can react with the strained cyclooctyne or azide, respectively, on the first phospholipid via Cu-free click chemistry. In some embodiments, the first derivatized phospholipid comprises the first phospholipid and the reaction product of a strained cyclooctyne and the targeting moiety comprising an azide.

For the copper-free click conjugation of targeting moieties to the liposomal surface, pre-modified with strained cyclooctynes, the targeting moieties are modified with azido moieties. For example, site-specific introduction of azide moieties to cetuximab can be achieved using a bioengineered Protein Z molecule, which binds only to Fc portions of IgG molecules (see, e.g., Hui, J. Z., Al Zaki, A., Cheng, Z., Popik, V., Zhang, H., Prak, E. T. L and Tsourkas, A. Facile Method for the Site-Specific, Covalent Attachment of Full-Length IgG onto Nanoparticles (2014) 10(16):3354-63. doi: 10.1002/sm11.201303629.) Stochastic introduction of azide moieties to cetuximab can be achieved using an N-hydroxysuccinimidyl ester of PEG4-azide. A single fluorophore (5-FAM for site-specific cetuximab and AlexaFluor® 488 for stochastic Cetuximab) can be introduced into each antibody conjugate. In some embodiments, micellized azido targeting ligands pre-clicked to strained cyclooctynes lipids can also be post-inserted into pre-formed liposomes carrying any therapeutic/imaging cargo. Targeting ligands can also be modified with a strained cyclooctyne for the click conjugation to azido-modified liposomes (e.g., a first derivatized phospholipid comprising a first phospholipid and an azido moiety).

The targeting moieties provided herein increase the selectivity of the liposomes provided herein. For example, selective binding and phototoxicity can be achieved for any click-conjugated targeting moiety with its respective target-overexpressing cell line. Azido modified ligands that are potential candidates for copper-free click conjugation to the liposomal platform include folate, RGD peptides, natural protein ligands (e.g., TNF, EGF, transferrin), affibodies, or any variant of antibody fragments. Selective binding and phototoxicity can also be achieved for any targeted cyclooctyne-modified liposomes encapsulating an aqueous photosensitizer (e.g., chlorin e6, methylene blue, sulfonated aluminium phthalocyanine, Rose Bengal). In some embodiments, water-soluble PEG-PLGA nanoparticles entrapping any hydrophobic photosensitizer can also be encapsulated within the targeted DIBO-modified liposomes for selective PDT-based modalities. Selective binding and phototoxicity can also be achieved for a liposome provided herein which further comprises an entrapped hydrophobic lipid-anchored photosensitizer such as protoporphyrin IX.

In some embodiments, the first derivatized phospholipid is present in an amount up to about 0.5 mol percent in the liposome. For example, the first derivatized phospholipid can be present in an amount of about 0.05 mol percent to about 0.5 mol percent (e.g., about 0.1 mol percent, about 0.2 mol percent, about 0.25 mol percent, about 0.3 mol percent, and about 0.4 mol percent).

A second phospholipid as described herein can be selected from the group consisting of: a phosphatidylcholine; a phosphatidic acid; a phosphatidylethanolamime; a phosphatidylglycerol; and a phosphatidylserine. Non-limiting examples of a second phospholipid include hydrogenated soy phosphotidylcholine (HSPC); 1,2-didecanoyl-sn-glycero-3-phosphocholine (DDPC); 1,2-dierucoyl-sn-glycero-3-phosphate (DEPA); 1,2-dielaidoyl-sn-glycero-3-phosphocholine (DEPC); 1,2-dierucoyl-sn-glycero-3-phosphoethanolamine (DEPE); 1, 2-dielaidoyl-phosphatidylglycerol (DEPG); 1,2-dilinoleoyl-sn-glycero-3-phosphocholine (DLOPC); 1,2-dilauroyl-sn-glycero-3-phosphate (DLPA); 1,2-dilauroyl-sn-glycero-3-phosphocholine (DLPC); 1,2-dilauroyl-sn-glycero-3-phosphoethanolamine (DLPE); 1,2-dilauroyl-sn-glycero-3-phosphatidylglycerol (DLPG); 1,2-dilauroyl-sn-glycero-3-phosphoserine (DLPS); 1,2-dimyristoyl-sn-glycero-3-phosphate (DMPA); 1,2-dimyristoyl-sn-glycero-3-phosphocholine (DMPC); 1,2-dimyristoyl-sn-glycero-3-phosphoethanolamine (DMPE); 1,2-dimyristoyl-sn-glycero-3-phosphatidylglycerol (DMPG); 1,2-dimyristoyl-sn-glycero-3-phosphoserine (DMPS); 1,2-dioleoyl-sn-glycero-3-phosphate (DOPA); 1,2-dioleoyl-sn-glycero-3-phosphocholine (DOPC); 1,2-dioleoyl-sn-glycero-3-phosphoethanolamine (DOPE); 1,2-dioleoyl-sn-glycero-3-phosphatidylglycerol (DOPG); 1,2-dioleoyl-sn-glycero-3-phosphoserine (DOPS); 1,2-dipalmitoyl-sn-glycero-3-phosphate (DPPA); 1,2-dipalmitoyl-sn-glycero-3-phosphocholine (DPPC); 1,2-dipalmitoyl-sn-glycero-3-phosphoethanolamine (DPPE); 1,2-dipalmitoyl-sn-glycero-3-phosphatidylglycerol (DPPG); 1,2-dipalmitoyl-sn-glycero-3-phosphoserine (DPPS); 1,2-distearoyl-sn-glycero-3-phosphate (DSPA); 1,2-distearoyl-sn-glycero-3-phosphocholine (DSPC); 1,2-distearoyl-sn-glycero-3-phosphoethanolamine (DSPE); 1,2-distearoyl-sn-glycero-3-phosphatidylglycerol (DSPG); 1,2-distearoyl-sn-glycero-3-phosphoserine (DSPS); 1-myristoyl-2-palmitoyl-sn-glycero 3-phosphocholine (MPPC); 1-myristoyl-2-stearoyl-sn-glycero-3-phosphocholine (MSPC); 1-palmitoyl-2-myristoyl-sn-glycero-3-phosphocholine (PMPC); 1-palmitoyl-2-oleoyl-sn-glycero-3-phosphocholine (POPC); 1-palmitoyl-2-oleoyl-sn-glycero-3-phosphoethanolamine (POPE); 1-palmitoyl-2-oleoyl-sn-glycero-3-phosphatidylglycerol (POPG); 1-palmitoyl-2-stearoyl-sn-glycero-3-phosphocholine (PSPC); 1-stearoyl-2-myristoyl-sn-glycero-3-phosphocholine (SMPC); and 1-stearoyl-2-oleoyl-sn-glycero-3-phosphocholine (SOPC); 1-stearoyl-2-palmitoyl-sn-glycero-3-phosphocholine (SPPC); and combinations thereof. In some embodiments, the second phospholipid is 1,2-distearoyl-sn-glycero-3-phosphoethanolamine (DSPE).

In some embodiments, the polyethylene glycol polymer present in the second derivatized phospholipid can have a molecular weight of about 350 g/mol to about 30,000 g/mol. For example, the polyethylene glycol polymer can have a molecular weight selected from the group consisting of 350 g/mol, 550 g/mol, 750 g/mol, 1000 g/mol, 2000 g/mol, 3000 g/mol, 5000 g/mol, 10,000 g/mol, 20,000 g/mol and 30,000 g/mol. In some embodiments, the polyethylene glycol polymer has a molecular weight of 2000 g/mol. In some embodiments, the polyethylene glycol polymer is terminated with an alkoxyl, carboxyl, amine, biotin, maleimide, succinyl, N-hydroxysuccinimidyl ester, sulfo-N-hydroxysuccinimidyl ester, silane, pyridyldithiol or cyanur moiety. In some embodiments, the second derivatized phospholipid is DSPE-PEG₂₀₀₀, DSPE-mPEG₂₀₀₀, or a combination thereof.

In some embodiments, the second derivatized phospholipid is present in an amount of about 0.5 mol percent to about 5 mol percent in the liposome. In some embodiments, the molar ratio of the conjugate to the second derivatized phospholipid is about 1:10. In some embodiments, the second derivatized phospholipid is present in an amount about 10-fold higher than the amount of the conjugate in the liposome.

Without being bound by any theory, the second derivatized phospholipid can impart stability to the liposome.

In some embodiments, the liposome comprises an anionic lipid. For example, the liposome can include an anionic phospholipid. Non-limiting examples of an anionic phospholipid include 1,2-dioleoyl-sn-glycero-3-phospho-(1′-rac-glycerol) (DOPG); phosphatidyl glycerol (PG); 1,2-dimyristoyl-sn-glycero-3-phospho-(1′-rac-glycerol) (DMPG); phosphatidylserine (PS); 1,2-dioleoyl-sn-glycero-3-phospho-L-serine (DOPS); and dicetylphosphate (DCP). An anionic lipid (e.g., an anionic phospholipid) can be present in an amount up to about 10 mol percent in the liposome (e.g., about 0.1 mol percent to about 10 mol percent). For example, the anionic lipid is present in an amount of about 5 to about 10 mol percent in the liposome. In some embodiments, the anionic lipid is present in an amount of about 7 to about 9 mol percent in the liposome.

In some embodiments, the liposome comprises a cationic lipid. For example, the liposome can include a cationic phospholipid. Non-limiting examples of a cationic phospholipid include 1,2-dioleoyl-3-trimethylammonium-propane (DOTAP). A cationic lipid (e.g., a cationic phospholipid) can be present in an amount up to about 10 mol percent in the liposome (e.g., about 0.1 mol percent to about 10 mol percent). For example, the anionic lipid is present in an amount of about 5 to about 10 mol percent in the liposome. In some embodiments, the anionic lipid is present in an amount of about 7 to about 9 mol percent in the liposome.

Without being bound by any theory, it is believed that the cationic or anionic lipid provides electrostatic stabilization of the liposome. For example, the lack of electrostatic stabilization can lead to precipitation of the liposomes. In some embodiments, the presence of a cationic or anionic lipid can contribute to increased cellular uptake of a liposome as provided herein as compared to a liposome lacking the cationic or anionic lipid.

In some embodiments, a liposome provided herein can further include a cargo selected from the group consisting of one or more chemotherapeutic compounds; one or more polymeric nanoparticles; one or more protein-based nanoparticles; one or more dendrimeric structures; one or more inorganic nanoparticles; one or more imaging agents; one or more kinase inhibitors; one or more biologics; and combinations of two or more thereof.

In some embodiments, the kinase inhibitor is a receptor tyrosine kinase inhibitor. The cargo can be biologics, (S. Tangutoori, B. Q. Spring, Z. Mai, A. Palanisami, L. Mensah and T. Hasan, Nanomedicine, 2015, DOI: 10.1016/j.nano.2015.08.007); chemotherapeutics (H. C. Huang, S. Mallidi, J. Liu, C. T. Chiang, Z. Mai, R. Goldschmidt, N. Ebrahim-Zadeh, I. Rizvi and T. Hasan, Cancer Res, 2016, 76, 1066-1077); or small molecule receptor tyrosine kinase inhibitors (B. Q. Spring, R. B. Sears, L. K. Zheng, Z. Mai, R. Watanabe, M. E. Sherwoord, D. A. Schoenfeld, B. W. Pogue, S. P. Pereira, E. Villa and T. Hasan, Nat. Nanotechnol., 2016, in press). In some embodiments, one or more of the one or more chemotherapeutic compounds exhibit synergy with the photosensitizer.

In some embodiments, the cargo can be entrapped in the liposomal bilayer (e.g., a hydrophobic chemotherapeutic or small molecule inhibitor).

A liposome provided herein can also include one or more phospholipids, sphingolipids, bioactive lipids, natural lipids, cholesterol, sterols or a combination thereof.

A phospholipid as described herein can be selected from the group consisting of: a phosphatidylcholine; a phosphatidic acid; a phosphatidylethanolamime; a phosphatidylglycerol; and a phosphatidylserine. Non-limiting examples of a phospholipid include hydrogenated soy phosphotidylcholine (HSPC); 1,2-didecanoyl-sn-glycero-3-phosphocholine (DDPC); 1,2-dierucoyl-sn-glycero-3-phosphate (DEPA); 1,2-dielaidoyl-sn-glycero-3-phosphocholine (DEPC); 1,2-dierucoyl-sn-glycero-3-phosphoethanolamine (DEPE); 1, 2-dielaidoyl-phosphatidylglycerol (DEPG); 1,2-dilinoleoyl-sn-glycero-3-phosphocholine (DLOPC); 1,2-dilauroyl-sn-glycero-3-phosphate (DLPA); 1,2-dilauroyl-sn-glycero-3-phosphocholine (DLPC); 1,2-dilauroyl-sn-glycero-3-phosphoethanolamine (DLPE); 1,2-dilauroyl-sn-glycero-3-phosphatidylglycerol (DLPG); 1,2-dilauroyl-sn-glycero-3-phosphoserine (DLPS); 1,2-dimyristoyl-sn-glycero-3-phosphate (DMPA); 1,2-dimyristoyl-sn-glycero-3-phosphocholine (DMPC); 1,2-dimyristoyl-sn-glycero-3-phosphoethanolamine (DMPE); 1,2-dimyristoyl-sn-glycero-3-phosphatidylglycerol (DMPG); 1,2-dimyristoyl-sn-glycero-3-phosphoserine (DMPS); 1,2-dioleoyl-sn-glycero-3-phosphate (DOPA); 1,2-dioleoyl-sn-glycero-3-phosphocholine (DOPC); 1,2-dioleoyl-sn-glycero-3-phosphoethanolamine (DOPE); 1,2-dioleoyl-sn-glycero-3-phosphatidylglycerol (DOPG); 1,2-dioleoyl-sn-glycero-3-phosphoserine (DOPS); 1,2-dipalmitoyl-sn-glycero-3-phosphate (DPPA); 1,2-dipalmitoyl-sn-glycero-3-phosphocholine (DPPC); 1,2-dipalmitoyl-sn-glycero-3-phosphoethanolamine (DPPE); 1,2-dipalmitoyl-sn-glycero-3-phosphatidylglycerol (DPPG); 1,2-dipalmitoyl-sn-glycero-3-phosphoserine (DPPS); 1,2-distearoyl-sn-glycero-3-phosphate (DSPA); 1,2-distearoyl-sn-glycero-3-phosphocholine (DSPC); 1,2-distearoyl-sn-glycero-3-phosphoethanolamine (DSPE); 1,2-distearoyl-sn-glycero-3-phosphatidylglycerol (DSPG); 1,2-distearoyl-sn-glycero-3-phosphoserine (DSPS); 1-myristoyl-2-palmitoyl-sn-glycero 3-phosphocholine (MPPC); 1-myristoyl-2-stearoyl-sn-glycero-3-phosphocholine (MSPC); 1-palmitoyl-2-myristoyl-sn-glycero-3-phosphocholine (PMPC); 1-palmitoyl-2-oleoyl-sn-glycero-3-phosphocholine (POPC); 1-palmitoyl-2-oleoyl-sn-glycero-3-phosphoethanolamine (POPE); 1-palmitoyl-2-oleoyl-sn-glycero-3-phosphatidylglycerol (POPG); 1-palmitoyl-2-stearoyl-sn-glycero-3-phosphocholine (PSPC); 1-stearoyl-2-myristoyl-sn-glycero-3-phosphocholine (SMPC); and 1-stearoyl-2-oleoyl-sn-glycero-3-phosphocholine (SOPC); 1-stearoyl-2-palmitoyl-sn-glycero-3-phosphocholine (SPPC); and a combination thereof. In some embodiments, at least one of the one or more phospholipids is 1,2-dipalmitoyl-sn-glycero-3-phosphocholine (DPPC).

In some embodiments, the one or more phospholipids are present at about 40 mol percent to about 80 mol percent in the liposome. For example, the one or more phospholipids are present at about 50 to about 70 mol percent in the liposome; about 55 to about 65 mol percent in the liposome; about 15 mol percent to about 45 mol percent in the liposome; or about 20 mol percent to about 30 mol percent in the liposome.

In some embodiments, the cholesterol is present in an amount of about 10 mol percent to about 50 mol percent of the liposome. For example, about 20 mol percent to about 40 mol percent of the liposome; about 25 to about 35 mol percent; and about 27 mol percent to about 29 mol percent of the liposome.

In some embodiments, a liposome provided herein comprises about 50 mol percent to about 65 mol percent of one or more phospholipids; about 5 mol percent to about 10 mol percent of an anionic or cationic lipid; about 20 to about 35 mol percent of cholesterol; about 2 mol percent to about 8 mol percent of a second derivatized phospholipid; and about 0.05 mol percent to about 0.25 mol percent of a conjugate. For example, a liposome provided herein comprises about 55 mol percent to about 60 mol percent of one or more phospholipids; about 7 mol percent to about 9 mol percent of an anionic or cationic lipid; about 25 to about 30 mol percent of cholesterol; about 4 mol percent to about 6 mol percent of a second derivatized phospholipid; and about 0.08 mol percent to about 0.12 mol percent of a conjugate.

In some embodiments, the liposome further comprises a fluorophore, a contrast agent (e.g., an MRI, PET, or SPECT contrast agent), or a combination thereof.

A “fluorophore”, as described herein, can be any small molecule that can re-emit light upon light excitation (e.g., light in the visible spectrum). For example, fluorophores can include rhodamines, fluoresceins, boron-dypyrromethanes, coumarins, pyrenes, cyanines, oxazines, acridines, auramine Os, and derivatives thereof. In some cases, derivatives include sulfonated derivatives such as sulfonated pyrenes, sulfonated coumarins, sulfonated rhodamines, and sulfonated cyanines (e.g., ALEXAFLUOR® dyes).

In some embodiments, lipid anchoring of any hydrophobic fluorophore containing chemically reactive functional groups such —OH, —COOH, —NH₂, —SH can be achieved through conjugation to lysophospholipids and lysophospholipid derivatives (e.g., lysophospholipids bound to a polyethylene glycol polymer). These liposomes can be click conjugated to any targeting moiety and will selectively bind to their targets. Lipid anchoring of any hydrophilic fluorophore containing chemically reactive functional groups such —OH, —COOH, —NH₂ and —SH can be achieved through conjugation to derivatives of the phosphate head groups of phospholipids such as DPPC, DPPE, DSPE-PEG-NH2, DSPE-PEG-COOH, DSPE-PEG-OH, DOPG or to the polar —OH group of cholesterol. These liposomes can be click conjugated to any targeting moiety and will selectively bind to their targets.

Liposomes as provided herein can be prepared using methods known to those having ordinary skill in the art. For example, the liposomes may be prepared in a suspension form or may be formed upon reconstitution of a lyophilized powder containing the liposome compositions provided herein with an aqueous solution. In some embodiments, the liposomes provided herein have a mean particle size distribution of less than 200 nm. For example, the liposomes provided herein can have a mean particle size distribution of about 100 nm to about 150 nm.

Also provided herein are pharmaceutical compositions comprising a liposome provided herein and a pharmaceutically acceptable excipient.

The liposomes provided herein can be administered either alone or in combination with a conventional pharmaceutical carrier, excipient or the like. Pharmaceutically acceptable excipients include, but are not limited to, surfactants used in pharmaceutical dosage forms such as Tweens, poloxamers or other similar polymeric delivery matrices, buffer substances such as phosphates, tris, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium-chloride, polyethylene glycol, and sodium carboxymethyl cellulose. Cyclodextrins such as α-, β, and γ-cyclodextrin, or chemically modified derivatives such as hydroxyalkylcyclodextrins, including 2- and 3-hydroxypropyl-β-cyclodextrins, or other solubilized derivatives can also be used. Dosage forms or compositions containing a liposome as described herein in the range of 0.005% to 100% with the balance made up from non-toxic carrier may be prepared. The contemplated compositions may contain 0.001%-100% of a liposome provided herein, in one embodiment 0.1-95%, in another embodiment 75-85%, in a further embodiment 20-80%. Actual methods of preparing such dosage forms are known, or will be apparent, to those skilled in this art; for example, see Remington: The Science and Practice of Pharmacy, 22^{nd} Edition (Pharmaceutical Press, London, U K. 2012).

Liquid pharmaceutically administrable compositions can, for example, be prepared by dissolving, dispersing, etc. a compound provided herein and optional pharmaceutical adjuvants in a carrier (e.g., water, saline, aqueous dextrose, glycerol, glycols, ethanol or the like) to form a solution, colloid, liposome, emulsion, complexes, coacervate or suspension. If desired, the pharmaceutical composition can also contain minor amounts of nontoxic auxiliary substances such as wetting agents, emulsifying agents, co-solvents, solubilizing agents, pH buffering agents and the like (e.g., sodium acetate, sodium citrate, cyclodextrin derivatives, sorbitan monolaurate, triethanolamine acetate, triethanolamine oleate, and the like).

Injectables can be prepared in conventional forms, either as liquid solutions, colloid, liposomes, complexes, coacervate or suspensions, as emulsions, or in solid forms suitable for reconstitution in liquid prior to injection. The percentage of a liposome provided herein contained in such parenteral compositions is highly dependent on the specific nature of the liposome and the needs of the patient.

It is to be noted that concentrations and dosage values may vary depending on the specific liposome and the severity of the condition to be alleviated. It is to be further understood that for any particular patient, specific dosage regimens should be adjusted over time according to the individual need and the professional judgment of the person administering or supervising the administration of the compositions.

Further provided herein are methods of treating a cancer in a patient, the method comprising administering to the patient a therapeutically effective amount of a liposome provided herein; and irradiating the patient to break the liposome.

The liposomes provided herein can also be used to image a cancer in a patient. In some embodiments, the methods include administering to the patient an effective amount of a liposome provided herein; irradiating the patient to break the liposome; and imaging the patient.

Non-Limiting Cancers Include, but are not Limited to, the Following:

1) Breast cancers, including, for example ER+ breast cancer, ER− breast cancer, her2− breast cancer, her2+ breast cancer, stromal tumors such as fibroadenomas, phyllodes tumors, and sarcomas, and epithelial tumors such as large duct papillomas; carcinomas of the breast including in situ (noninvasive) carcinoma that includes ductal carcinoma in situ (including Paget's disease) and lobular carcinoma in situ, and invasive (infiltrating) carcinoma including, but not limited to, invasive ductal carcinoma, invasive lobular carcinoma, medullary carcinoma, colloid (mucinous) carcinoma, tubular carcinoma, and invasive papillary carcinoma; and miscellaneous malignant neoplasms. Further examples of breast cancers can include luminal A, luminal B, basal A, basal B, and triple negative breast cancer, which is estrogen receptor negative (ER−), progesterone receptor negative, and her2 negative (her2−). In some embodiments, the breast cancer may have a high risk Oncotype score.

2) Cardiac cancers, including, for example sarcoma, e.g., angiosarcoma, fibrosarcoma, rhabdomyosarcoma, and liposarcoma; myxoma; rhabdomyoma; fibroma; lipoma and teratoma.

3) Lung cancers, including, for example, bronchogenic carcinoma, e.g., squamous cell, undifferentiated small cell, undifferentiated large cell, and adenocarcinoma; alveolar and bronchiolar carcinoma; bronchial adenoma; sarcoma; lymphoma; chondromatous hamartoma; and mesothelioma.

4) Gastrointestinal cancer, including, for example, cancers of the esophagus, e.g., squamous cell carcinoma, adenocarcinoma, leiomyosarcoma, and lymphoma; cancers of the stomach, e.g., carcinoma, lymphoma, and leiomyosarcoma; cancers of the pancreas, e.g., ductal adenocarcinoma, insulinoma, glucagonoma, gastrinoma, carcinoid tumors, and vipoma; cancers of the small bowel, e.g., adenocarcinoma, lymphoma, carcinoid tumors, Kaposi's sarcoma, leiomyoma, hemangioma, lipoma, neurofibroma, and fibroma; cancers of the large bowel, e.g., adenocarcinoma, tubular adenoma, villous adenoma, hamartoma, and leiomyoma.

5) Genitourinary tract cancers, including, for example, cancers of the kidney, e.g., adenocarcinoma, Wilm's tumor (nephroblastoma), lymphoma, and leukemia; cancers of the bladder and urethra, e.g., squamous cell carcinoma, transitional cell carcinoma, and adenocarcinoma; cancers of the prostate, e.g., adenocarcinoma, and sarcoma; cancer of the testis, e.g., seminoma, teratoma, embryonal carcinoma, teratocarcinoma, choriocarcinoma, sarcoma, interstitial cell carcinoma, fibroma, fibroadenoma, adenomatoid tumors, and lipoma.

6) Liver cancers, including, for example, hepatoma, e.g., hepatocellular carcinoma; cholangiocarcinoma; hepatoblastoma; angiosarcoma; hepatocellular adenoma; and hemangioma.

7) Bone cancers, including, for example, osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochrondroma (osteocartilaginous exostoses), benign chondroma, chondroblastoma, chondromyxofibroma, osteoid osteoma and giant cell tumors.

8) Nervous system cancers, including, for example, cancers of the skull, e.g., osteoma, hemangioma, granuloma, xanthoma, and osteitis deformans; cancers of the meninges, e.g., meningioma, meningiosarcoma, and gliomatosis; cancers of the brain, e.g., astrocytoma, medulloblastoma, glioma, ependymoma, germinoma (pinealoma), glioblastoma multiform, oligodendroglioma, schwannoma, retinoblastoma, and congenital tumors; and cancers of the spinal cord, e.g., neurofibroma, meningioma, glioma, and sarcoma.

9) Gynecological cancers, including, for example, cancers of the uterus, e.g., endometrial carcinoma; cancers of the cervix, e.g., cervical carcinoma, and pre tumor cervical dysplasia; cancers of the ovaries, e.g., ovarian carcinoma, including serous cystadenocarcinoma, mucinous cystadenocarcinoma, unclassified carcinoma, granulosa theca cell tumors, Sertoli Leydig cell tumors, dysgerminoma, and malignant teratoma; cancers of the vulva, e.g., squamous cell carcinoma, intraepithelial carcinoma, adenocarcinoma, fibrosarcoma, and melanoma; cancers of the vagina, e.g., clear cell carcinoma, squamous cell carcinoma, botryoid sarcoma, and embryonal rhabdomyosarcoma; and cancers of the fallopian tubes, e.g., carcinoma.

10) Hematologic cancers, including, for example, cancers of the blood, e.g., acute myeloid leukemia, chronic myeloid leukemia, acute lymphoblastic leukemia, chronic lymphocytic leukemia, myeloproliferative diseases, multiple myeloma, and myelodysplastic syndrome, Hodgkin's lymphoma, non-Hodgkin's lymphoma (malignant lymphoma) and Waldenström's macroglobulinemia.

11) Skin cancers and skin disorders, including, for example, malignant melanoma and metastatic melanoma, basal cell carcinoma, squamous cell carcinoma, Kaposi's sarcoma, moles dysplastic nevi, lipoma, angioma, dermatofibroma, keloids, and scleroderma.

12) Adrenal gland cancers, including, for example, neuroblastoma.

Cancers may be solid tumors that may or may not be metastatic. Cancers may also occur, as in leukemia, as a diffuse tissue. Thus, the term “tumor cell,” as provided herein, includes a cell afflicted by any one of the above identified disorders.

A method of treating cancer using a compound or composition as described herein may be combined with existing methods of treating cancers, for example by chemotherapy, irradiation, or surgery (e.g., oophorectomy). In some embodiments, a compound or composition can be administered before, during, or after another anticancer agent or treatment.

A therapeutic effect relieves, to some extent, one or more of the symptoms of the disease.

“Treat,” “treatment,” or “treating,” as used herein refers to administering a compound or pharmaceutical composition as provided herein for therapeutic purposes. The term “therapeutic treatment” refers to administering treatment to a patient already suffering from a disease thus causing a therapeutically beneficial effect, such as ameliorating existing symptoms, ameliorating the underlying metabolic causes of symptoms, postponing or preventing the further development of a disorder, and/or reducing the severity of symptoms that will or are expected to develop.

The liposomes provided herein are labile to photoxidation. In some embodiments, the liposomes provided herein can be used for light-triggered release of liposomal payloads. See, for example, B. Q. Spring, R. B. Sears, L. K. Zheng, Z. Mai, R. Watanabe, M. E. Sherwoord, D. A. Schoenfeld, B. W Pogue, S. P Pereira, E. Villa and T. Hasan, Nat. Nanotechnol., 2016, in press. For activation of the photosensitizer of the liposomes provided herein, any suitable absorption wavelength is used. This absorption wavelength can be supplied using the various methods known to the art for mediating cytotoxicity or fluorescence emission, such as visible radiation, including incandescent or fluorescent light sources or photodiodes, such as light emitting diodes. Laser light is also used for in situ delivery of light to the localized liposomes.

In some embodiments, a liposome as provided herein is imaged in vivo using fluorescent imaging. For example, the use of such methods permits the facile, real-time imaging and localization of cells or tissues labeled with a liposome provided herein. In some embodiments, a liposome provided herein is imaged in vivo using laparoscopy and/or endomiscroscopy. For example, the use of laparoscopy permits the facile, real-time imaging and localization of cells or tissues labeled with a liposome provided herein. In some embodiments, a liposome can be imaged using fiber optic endomicroscopy.

A number of preclinical and clinical applications for a liposome provided herein can be envisioned. For example, a liposome described here can be used: 1) for the early detection cancers; 2) as an aid to surgeons during surgery (e.g., by allowing for real-time detection of cancer cells); and 3) as a method for monitoring the progress of a cancer treatment (e.g., by quantifying the cancer cells present before, during, and after treatment).

For example, the liposomes provided herein can be administered to a subject in combination with surgical methods, for example, resection of tumors. The liposomes can be administered to the individual prior to, during, or after surgery. The liposomes can be administered parenterally, intravenous or injected into the tumor or surrounding area after tumor removal, e.g., to image or detect residual cancer cells. For example, the liposomes can be used to detect the presence of a tumor and to guide surgical resection. In some embodiments, the liposomes can be used to detect the presence of residual cancer cells and to guide continued surgical treatment until at least a portion (e.g., all) such cells are removed from the subject. Accordingly, there is provided a method of guided surgery to remove at least a portion of a tumor from a subject comprising providing a liposome provided herein; causing the liposome to be present in at least some cancer cells; observing the image following activation of the liposome (e.g., fluorescence); and performing surgery on the subject to remove at least a portion of the tumor that comprises detected cancer cells.

With respect to in vitro imaging methods, the liposomes and compositions described herein can be used in a variety of in vitro assays. An exemplary in vitro imaging method comprises: contacting a sample, for example, a biological sample (e.g., a cell), with one or more liposomes provided herein; allowing the liposomes to interact with a biological target in the sample; illuminating the sample with light of a wavelength absorbable by the photosensitizers or imaging agents.

Stable anchoring of hydrophilic fluorophores can be achieved through conjugation of the fluorophore to cholesterol, to the phosphate head group of phospholipids like 1,2-dipalmitoyl-sn-glycero-3-phosphoethanolamine (DPPE) or to the terminal end of PEGylated phospholipids such as DSPE-PEG₂₀₀₀-NH2.

The high chemoselectivity of copper-free click chemistry enables the modular conjugation of different reactive entities to the surface of the liposomes without cross-reactivity with the click chemistry reactants (e.g., the strained cyclooctyne or the azide moiety). For example, a liposome containing 0.2% DSPE-PEG₂₀₀₀-NH2 and DIBO-modified lysophospholipids were used to conjugate amine-reactive NHS-esters of near-infrared dyes to the terminal amine of the DSPE-PEG₂₀₀₀-NH2 to prepare a panel of stable click-reactive liposomes capable of deep tissue in vivo imaging. Such dyes can include AlexaFluor® 633, AlexaFluor® 647, AlexaFluor® 680, AlexaFluor®700, IRDye® 680RD and IRDye® 800CW. Liposomes as provided herein can contain 16:0 LysoPC-BPD (or any other lipid-anchored hydrophobic photosensitizer for photodynamic therapy) in the membrane prior to reaction of amine-containing lipids with amine-reactive dyes without affecting fluorophore labeling, conjugation of targeting moiety, or selectivity. The liposomes provided herein can also contain aqueous therapeutic agents in the core prior to reaction of amine-containing lipids with amine-reactive dyes without affecting fluorophore labeling, conjugation of targeting moiety or selectivity.

Administration of the liposomes disclosed herein can be via any of the accepted modes of administration. In some embodiments, the liposomes are administered parenterally (e.g., intravenously).

### Examples

### Materials and Methods

### Coupling of BPD Photosensitizer to Phospholipids 16:0 Lyso PC and 20:0 Lyso PC

The carboxylate of the benzoporphyrin derivative photosensitizer was coupled to the hydroxyl moiety of the phospholipid 1-palmitoyl-2-hydroxy-sn-glycero-3-phosphocholine (16:0 Lyso PC) or the phospholipid 1-arachidoyl-2-hydroxy-sn-glycero-3-phosphocholine (20:0 Lyso PC) through an esterification, according to a modified synthetic protocol. See J. Lovell, C. Jin, E. Huynh, H. Jin, C. Kim, J. Rubinstein, W. Chan, W. Cao, L. Wang and G. Zheng. Porphysome nanovesicles generated by porphyrin bilayers for use as multimodal biophotonic contrast agents 2011 Nature Materials, 10, 324-332. 16:0 Lyso PC (99.13 μl, 25 mg/ml stock in chloroform) or 20:0 Lyso PC (110.35 μl, 25 mg/ml stock in chloroform) was placed in a 13×100 mm Pyrex® tube and the chloroform was evaporated using a flow of nitrogen gas through a 16 gauge needle. The photosensitizer benzoporphyrin derivative monoacid ring A (BPD, verteporfin, 17.97 mg, 718.79 g/mol) was added to the dried 16:0 Lyso PC or 20:0 Lyso PC. 1-ethyl-3-(3-dimethylaminopropyl) carbodiimide (EDC, 38.81 mg, 155.24 g/mol; Sigma-Aldrich), 4-(dimethylamino)pyridine (DMAP, 15.27 mg, 122.17 g/mol; Sigma-Aldrich), and N,N-diisopropylethylamine (DIPEA, 52.25 μl, 129.24 g/mol. 0.742 g/ml; Sigma-Aldrich) were then added to the dried mixture of 16:0 Lyso PC or 20:0 Lyso PC with the BPD mixture. The molar ratio of 16:0 Lyso PC/20:0 Lyso PC:BPD:EDC:DMAP:DIPEA was 1:5:50:25:300. The mixtures was dissolved in dichloromethane (DCM, 5 ml) and rigorously stirred at 2500 rotations per minute for 24 hours at room temperature in the dark using a magnetic stir plate. The 16:0 Lyso PC-BPD and 20:0 Lyso PC-BPD lipid conjugates were purified using Analtech Preparative Thin Layer Chromatography Silica Uniplates eluting with 10% methanol in DCM. The most polar 16:0 Lyso PC-BPD or 20:0 Lyso PC-BPD-containing silica fraction (Rf˜0.144) was removed from the TLC plate and placed in a 50 ml polypropylene tube. The 16:0 Lyso PC-BPD or 20:0 Lyso PC-BPD was extracted from the silica fraction by sonication in 33% methanol in DCM (30 ml) for 10 min. The silica was sedimented by centrifugation at 3,700×g for 10 min and the supernatant containing the extracted 16:0 Lyso PC-BPD or 20:0 Lyso PC-BPD was collected in a 250 ml round-bottom flask. The silica fraction was washed with 33% methanol in DCM two additional times and all 16:0 Lyso PC-BPD or 20:0 Lyso PC-BPD solutions were separately combined into the 250 ml round bottom flask. The solvent mixtures were removed from the extracted 16:0 Lyso PC-BPD or 20:0 Lyso PC-BPD by rotary evaporation under reduced pressure at 40° C. connected to a liquid nitrogen trap condenser. Residual silica that previously dissolved in the methanol-DCM solvent mixture was removed by redissolving the dried 16:0 Lyso PC-BPD or 20:0 Lyso PC-BPD extracts in 100% DCM. The insoluble silica precipitate was removed by filtration using a Fisherbrand™ poly(tetrafluoroethylene) (PTFE) filter (0.22 μm pore size, 13 mm diameter) affixed to the end of a polypropylene syringe. The DCM was removed from the filtered 16:0 Lyso PC-BPD or 20:0 Lyso PC-BPD solutions using rotary evaporation. The purified conjugates were then redissolved in chloroform (5 ml) and stored in the dark at −20° C. The concentrations of the 16:0 Lyso PC-BPD or 20:0 Lyso PC-BPD were determined by diluting the phospholipid conjugate in DMSO and measuring the UV-Visible absorption spectrum using an extinction coefficient ε_{687 nm} of 34,895 M⁻¹·cm⁻¹.

### Liposome Synthesis

All liposome formulations were prepared using hydrated lipid film processes. Lipid films were initially prepared in 13×100 mm Pyrex® tubes from chloroform solutions of all lipids and dopants. Lipid mixtures were prepared from 1,2-dipalmitoyl-sn-glycero-3-phosphocholine (DPPC, 734.04 g/mol), 1,2-dioleoyl-3-trimethylammonium-propane (chloride salt) (DOTAP, cationic, 698.54 g/mol) or 1,2-di-(9Z-octadecenoyl)-sn-glycero-3-phospho-(1′-rac-glycerol) (sodium salt) (DOPG, anionic, 797.026 g/mol), cholesterol (386.65 g/mol), 1,2-distearoyl-sn-glycero-3-phosphoethanolamine-N-[methoxy(polyethyleneglycol)-2000] (ammonium salt) (DSPE-mPEG₂₀₀₀, 2805.50 g/mol) and 1,2-distearoyl-sn-glycero-3-phosphoethanolamine-N-[dibenzocyclooctyl(polyethylene glycol)-2000] (ammonium salt) (DSPE-PEG₂₀₀₀-ADIBO, 3077.80 g/mol). All of the foregoing reagents were purchased from Avanti® Polar Lipids, Inc., and were prepared to a total of 34.043 μmol lipid. DPPC, DOTAP(cationic)/DOPG(anionic), cholesterol, DSPE-mPEG₂₀₀₀ and DSPE-PEG₂₀₀₀-ADIBO were mixed at mole percent ratios of 58.2:7.9:28.9:4.5:0.5, respectively, unless otherwise stated elsewhere. PEGylated DSPE was consistently kept at a total of 5%, with 0.5% substituted for DSPE-PEG₂₀₀₀-ADIBO to mediate the copper-free click conjugation to azide-derivatized antibodies. In one experiment, the mole percent of PEGylated DSPE was varied to 5%, 4%, 3%, 2%, 1% and 0.5% whilst retaining a 1:10 ratio of DSPE-PEG₂₀₀₀-ADIBO:DSPE-mPEG₂₀₀₀. The photosensitizer-phospholipid conjugates, 16:0 Lyso PC-BPD or 20:0 Lyso PC-BPD, replaced a portion of the DPPC 200 nmol (0.6 mol %). For liposomal formulations requiring free BPD hydrophobic incorporation, a chloroform solution of unconjugated BPD was added to the lipid mixture in chloroform. All lipids were briefly vortexed and the chloroform was evaporated using a flow of nitrogen gas through a 16-gauge needle with continuous rotation to form a thin lipid film. Residual chloroform was removed by storing the lipid film under vacuum for 24 h. The dried lipid film was hydrated using 1×phosphate buffered saline (PBS) using the freeze-thaw-vortex method. 1×DPBS (1 ml) was added to the lipid film and the Pyrex® tube was tightly sealed and wrapped in Parafilm®. For experiments with aqueous photosensitizers such as the chlorin e6 monoethylene diamine monoamide (CMA), lipid film void 16:0 Lyso PC-BPD was hydrated with 1 ml of PBS containing 400 μM CMA and 1% PEG₃₀₀ as an excipient to prevent CMA stacking. The tube was then incubated in a darkened water bath at 42° C. for 10 min, vortexed at maximum speed for 30 seconds, then incubated in an ice bath for 10 min. The cycle was repeated a further 4 times to prepare multilamellar vesicles. To prepare monodisperse unilamellar liposomes, the multilamellar vesicle suspensions (1 ml) were extruded 5 times at 42° C. through two polycarbonate membranes, (0.1 μm pore size, 19 mm diameter) using an Avanti® Mini-Extruder kit. The liposomes were stored at 4° C. in a darkened container. The concentration of the 16:0 Lyso PC-BPD within the liposomal formulations was determined by diluting and homogenizing the liposomes in DMSO and measuring the UV-Visible absorption spectrum, using an extinction coefficient ε_{687 nm} of 34,895 M⁻¹·cm⁻¹.

### Phototriggered Release of Liposomal Payload

Liposomes containing optimal DSPE-PEG₂₀₀₀-ADIBO concentrations and either 200 nmol of 16:0 Lyso PC-BPD or no photosensitizer as a control were prepared as described above but hydrated with 1 ml of PBS containing 100 mM calcein disodium salt. The liposomes were extruded 5 times at 42° C. through two polycarbonate membranes, (0.1 μm pore size, 19 mm diameter) as described above. Unencapsulated calcein disodium salt was removed by dialysis of the 1 ml liposome aliquots in Float-A-Lyzer® dialysis tubes (300 kDa molecular weight cut-off) against 1×PBS at 4° C. for 48 hours. The liposomes were then run through illustra NAP Columns pre-packed with Sephadex G-25 to remove residual extra-liposomal calcein disodium salt. The concentration of calcein disodium salt was measured using ε_{492 nm} of 70,000 M⁻¹·cm⁻¹ and diluted to 2 μM in 1×PBS or 1×PBS containing 10 mM sodium azide as a quencher of reactive molecular species. The liposomes were irradiated using a 690 nm diode laser at an irradiance of 150 mW/cm² for incrementally increasing fluences up to 100 J/cm². Release of the calcein surrogate of liposomal payload was measured as a function of the degree of fluorescence dequenching, which was measured by a plate reader using a 450 nm excitation filter, 475 nm cut-off, and an emission profile from 500-70 nm.

### Site-Specific Protein Z Conjugation to Cetuximab

Site-specific conjugation of Protein Z to Cetuximab was performed according to a previously published protocol. See J. Hui, S. Tamsen, Y. Song and A. Tsourkas. LASIC: Light Activated Site-Specific Conjugation of Native IgGs, 2015 Bioconjugate Chemistry, 26, 1456-1460. The bioengineered protein Z molecule contained the unnatural amino acid benzoylphenylalanine (BPA) and the IgG binding site to mediate covalent cross-linking through 365 nm UV photocross-linking. The Protein Z construct also contained a terminal custom peptide with a 5-carboxyfluorescein molecule (5-FAM) and an azide moiety for click chemistry. The concentration of Cetuximab was determined using UV-visible spectrophotometry and an extinction coefficient ε_{280 nm} of 217,315 M⁻¹·cm⁻¹ (information obtained using Expasy Protoparam Tools). The concentration of 5-FAM corresponding to the site-specifically conjugated Protein Z molecule was determined using UV-Visible spectrophotometry and an extinction coefficient ε_{492 nm} of 82,000 M⁻¹·cm⁻¹. The purified azido Cetuximab-Protein Z was stored in the dark at 4° C. until needed for click conjugation to the liposomes.

### Stochastic Azido Modification to Antibodies and Targeting Moieties

An azido moiety was stochastically introduced to the Cetuximab through the stochastic conjugation of N-hydroxysuccinimidyl azido poly(ethylene glycol)4 (NHS-PEG4-Azide, 388.37 g/mol) to the antibody's lysine residues. Simultaneously, Cetuxumab was stochastically conjugated to the N-hydroxysuccinimidyl ester of Alexa Fluor® 488 (AF488-NHS, 643.4 g/mol). Stock solutions of NHS-PEG4-Azide (10 mg/ml) and AF488-NHS (1 mg/ml) in anhydrous dimethyl sulfoxide (DMSO) were both mixed at quantities corresponding to a 2.5-fold molar excess of each molecule to the Cetuximab prior to the addition of the antibody solution. Cetuximab (2 mg/ml in 1×DPBS, 145781.6 g/mol (FASTA sequence analysis), Erbitux®; Bristol-Myers Squibb) was then added to the NHS-PEG4-Azide and AF488-NHS mixture and was mixed by orbital rotation for 24 h at 4° C. in the dark. Unreacted NHS-PEG4-Azide and AF488-NHS were removed from the conjugated Cetuximab by illustra NAP Columns pre-packed with Sephadex G-25 and equilibrated with 1×DPBS. The purified Cetuximab conjugated to AF488 and PEG4-Azide (AF488-Cet-PEG4-Azide) was collected and concentrated by centrifuging in 30 kDa ultrafiltration tubes at 2,500×g for 20 min at 4° C. The concentration of Cetuximab was determined using UV-visible spectrophotometry and an extinction coefficient E280 nm of 217,315 M⁻¹·cm⁻¹ (information obtained using Expasy Protoparam Tools). The concentration of Alexa Fluor® 488 conjugated to the AF488-Cet-PEG4-Azide construct was determined using UV-visible spectrophotometry and an extinction coefficient ε_{494 nm} of 71,000 M⁻¹·cm⁻¹. The purified AF488-Cet-PEG4-Azide was stored in the dark at 4° C. until needed for click conjugation to the liposomes. The same approach was taken for the anti-HER-2 antibody, Trastuzumab, and transferrin, the natural ligand for the transferrin receptor. All stochastic azido ligands were stored in the dark at 4° C. until needed for click conjugation to the liposomes.

### Copper-Free Click Conjugation to Liposomes

All liposomes were conjugated site-specifically to Cetuximab or stochastically to Cetuximab, Trastuzumab by copper-free click conjugation of the liposomal surface ADIBO with the azido functionality on the targeting moieties. The click conjugation between the liposomes and the targeting moieties was performed overnight at room temperature. Excess targeting moieties were removed from the click conjugated liposomes by size exclusion Sepharose CL-4B gel filtration chromatography and were stored in the dark at 4° C. following full physical and spectroscopic characterization.

### Dynamic Light Scattering and Potential Characterization

Z-average diameters, polydispersity indices and potential of all liposomes following each modification were measured using a Zetasizer Nano ZS (Malvern Instruments Ltd.). For simultaneous z-average diameter and polydispersity index measurements, 2 μl of liposomes were placed in a 4 ml polystyrene 4× optical cuvette and 1 ml of 1×DPBS was added to the liposomes. Temperature equilibration for a duration of 120 seconds was performed prior to the three individual measurements performed for each sample. ζ potential measurements were performed using a Folded Zeta Capillary Cell. 10 μl of liposomes were diluted with 1 ml of 3 mM NaCl, loaded into the cell and three individual measurements were performed for each sample.

### Flow Cytometry for Selectivity of Cellular Binding

All flow cytometry analyses were performed using BD FACSAria following the same procedure. Briefly, 75% confluent monolayer cell cultures were washed once in 1×PBS (5 ml), once in 1×PBS containing 5 mM ethylenediaminetetraacetic acid (EDTA) and 5 mM ethylene glycol tetraacetic acid (EGTA), and incubated for 15 minutes in fresh EDTA and EGTA-containing 1×PBS (5 ml) at 37° C. with frequent agitation. The trypsin-free cell suspensions were then centrifuged at 1000 rotations per minute for 5 minutes, the EDTA and EGTA containing 1×PBS was aspirated, and the cells were redispersed in their respective culture media and counted using a Coulter counter. 50,000 cells were placed into individual 1.5 ml centrifuge tubes for each condition and were centrifuged at 1000×g for 5 minutes to pellet the cells. The media was aspirated and the cell pellets were redispersed in 100 μl of their respective culture media including the liposome formulations to be tested at its respective concentration. Following incubation at 37° C. for time durations of 30 min, 90 min or 180 min, the cells were centrifuged at 1000×g for 5 minutes to pellet the cells, the media was aspirated, and the cell pellets were redispersed in 200 μl of ice cold PBS. The cells were kept on ice until BD FACSAria cytometry analysis was performed. BPD fluorescence emission from the cells was detected using excitation from a 405 nm laser, 5-carboxyfluorescein (5-FAM) and AlexaFluor® 488 emission from the cells was achieved using excitation from a 488 nm laser, and Lissamine Rhodamine B emission from the cells was achieved using excitation from a 488 nm laser. Competitive blocking assays were performed using 1 mg/ml final concentration of free Cetuximab during liposome incubation with the cells.

### Transmission Electron Microscopy

ADIBO-modified liposomes were prepared as described previously containing either 200 nmol 16:0 Lyso PC-BPD or 0.1% DPPE-Lissamine Rhodamine B. Both liposomes were site-specifically click conjugated to Cetuximab-Protein Z, purified using Sepharose CL-4B gel filtration chromatography and concentrated using ultrafiltration centrifugation using a 30 kD molecular weight cut-off 4 ml cellulose ultrafiltration tube centrifuged at 3000×g for 30 minutes at room temperature. 10 μl of concentrated liposomes were loaded onto copper mesh carbon coated grid for 1 minute, followed by staining with phosphotungstic acid for 10 seconds. The samples were dried overnight then imaged at an accelerating voltage of 80 kV and a magnification of 46,000×using a Philips CM10 Transmission Electron Microscope (TEM).

### Confocal Microscopy

OVCAR-5 (High EGFR) cells were seeded onto glass bottom 96 well plates for 48 hours in serum-containing RPMI media at a density of 2000 cells per well. The media was replaced with fresh serum-containing RPMI media with 100 nM 16:0 Lyso PC-BPD equivalent of stochastically conjugated or non-conjugated liposomes. The cells were imaged using confocal fluorescence microscopy at 1 hour, 6 hours and 24 hours following incubation using A Leica TCS NT confocal microscope.

### Cellular Uptake Studies

Cells of interest were seeded in their respective serum-containing media for 24 hours in 96 well plates at a density of 100,000 cells per well. The media was replaced with fresh media containing desired liposomes of different charges and was incubated at 37° C. At the required time-point, the media containing liposomes was aspirated, the cells were washed 3 times in 100 μl of 1×PBS then lysed in Solvable® tissue digestion solution. A 25 μl aliquot was taken and used to quantify the cell protein concentration in each well using the colorimetric BCA® Assay. The remaining cell digest was used to quantify internalized Lyso PC-BPD concentrations using fluorescence, which was ultimately normalized to the derived protein concentrations.

### Targeted Phototoxicity In Vitro

Anionic liposomes containing 200 nmol of 16:0 Lyso PC-BPD and the optimized amount of DSPE-PEG₂₀₀₀-ADIBO were synthesized as described above and click conjugated to either 114 site-specific Cetuximab-Protein Z molecules per liposome or 11 stochastic Cetuximab molecules per liposome. Unconjugated antibodies were purified using Sepharose CL-4B gel filtration chromatography and the concentration of 16:0 Lyso PC-BPD was determined using a Thermo Fisher UV-visible spectrophotometer. All liposomes were stored at 4° C. in the dark prior to use.

A431 human epidermoid carcinoma cells (high EGFR) and wild-type Chinese hamster ovary cells (CHO-WT, EGFR null) were seeded on transparent bottom, black walled 96 well plates at a density of 3000 cells per well in serum-containing E-MEM media (A431 cells) and 2500 cells per well in serum-containing F12K media (CHO-WT). 24 hours following seeding, the media in the well plates was replaced with fresh media containing desired concentrations of liposomes site-specifically or stochastically click conjugated to Cetuximab and incubated for 3 hours. The media containing liposomes was then replaced with fresh media and the cells were irradiated with a 690 nm diode laser at an irradiance of 150 mW/cm² and a fluence of 40 J/cm². 24 hours after irradiation, the media was replaced with fresh media containing 3-(4,5-dimethylthiazol-2-yl)-2,5-diphenyltetrazolium Bromide (MTT) and the cells were incubated for 1 hour at 37° C. The media was then removed, the cells and formazan MTT product were dissolved with DMSO, and the absorbance was measured at 576 nm using a plate reader. Cell viability was depicted as a function of metabolic activity, normalized to untreated control cells.

### Modular Near-Infrared Dye Labeling of Liposomal Platform

Anionic lipid films were prepared as described above with 4.3% DSPE-mPEG₂₀₀₀, 0.5% DSPE-PEG₂₀₀₀-ADIBO and 0.2% DSPE-PEG₂₀₀₀-NH2 in the absence of the photosensitizing 16:0 Lyso PC-BPD conjugate. The lipid films were hydrated in 1×PBS and extruded through two 100 nm polycarbonate membranes as described above. To directly conjugate the near infrared dyes to amino-terminal DSPE-PEG₂₀₀₀-NH2, DMSO solutions (10 mg/ml) of N-hydroxysuccinimidyl (NHS) esters of the dyes AlexaFluor® 633, AlexaFluor® 647, AlexaFluor® 680, AlexaFluor®700, IRDye® 680RD and IRDye® 800CW were reacted with the liposomes at a 5-fold molar excess to the surface-accessible amino phospholipids. Briefly, the 0.034 μmoles of surface-accessible DSPE-PEG₂₀₀₀-NH2 moieties in 1 ml of the liposomal formulation were reacted with a 5-fold molar excess (0.171 μmoles) of dye NHS esters. The liposome dye mixtures were stirred at 2500 rotations per minute using a magnetic stirrer for 24 hours at room temperature, and then dialyzed against 1×PBS at 4° C. for 48 hours. Finally, the liposomes were click conjugated to 50 stochastic azido Cetuximab molecules per liposome or 50 azido IgG molecules per liposome for 24 hours at room temperature through the surface DSPE-PEG₂₀₀₀-ADIBO moieties. Unconjugated antibodies were purified using Sepharose CL-4B gel filtration chromatography and the concentration of near infrared dyes conjugated to the liposomes was determined using a Thermo Fisher UV-visible spectrophotometer. All liposomes were stored at 4° C. in the dark.

### Dynamic Dual Tracer Imaging In Vivo

ADIBO-modified liposomes modularly labeled with IRDye®680RD or IRDye®800CW were stochastically click conjugated to 50 Cetuximab molecules per liposome or 50 IgG molecules per liposome, respectively. Following purification using Sepharose CL-4B gel filtration chromatography, the concentration of IRDye®680RD or IRDye®800CW was determined using a Thermo Fisher UV-visible spectrophotometer. Female Swiss nude mice (6 weeks old) were implanted subcutaneously on the lower left flank with 1×10⁶ U251 human glioblastoma cells (High EGFR) in growth factor reduced Matrigel and allowed to grow for two weeks. The mice were co-injected with a mixture of Cetuximab targeted IRDye®680RD liposomes and IgG non-targeted IRDye®800CW liposomes at an amount of 0.1 nmol dye equivalent per liposomal conjugate. The relative tumoral fluorescence intensities were dynamically and longitudinally monitored for both the targeted IRDye®680RD liposomes and IgG non-targeted IRDye®800CW control liposomes using the Pearl® Impulse Small Animal Imaging System. Tumors were imaged up to 120 hours following co-administration of the dual tracer nanoplatforms.

### Biodistribution of Targeted Photosensitizing Liposomal Platform

6-Week old female Swiss nude mice were implanted subcutaneously on the lower right flank with 1×10⁶ A431 human epidermoid carcinoma cells (High EGFR) in growth factor reduced Matrigel and the cells allowed to grow for two weeks. The mice were then injected with 0.25 mg/kg BPD equivalent of 16:0 Lyso PC-BPD liposomes either (1) click conjugated to 10 stochastic Cetuximab molecules per liposome or (2) unconjugated. 24 hours following intravenous administration, the animals were sacrificed and the organs were imaged using an IVIS® Lumina III in vivo preclinical imaging station. The fluorescence intensity of 16:0 Lyso PC-BPD liposomes was analyzed and quantified using the Living Image® Software and intensities in tumors were normalized to respective intensities in organs to quantify tissue selectivity.

### Optimization Studies

### 16:0 Lyso PC Lipid-Anchored Benzoporphyrin Derivative Photosensitizer (16:0 Lyso PC-BPD)

It has been observed that photosensitizer or fluorophore leeches from the membrane of nanoliposomal carriers if the photosensitizer is not stably fixed to the liposome, resulting in a reduced selectivity. Stable anchoring of hydrophobic fluorophores or photosensitizers into the membrane can be achieved by conjugating the fluorophores or photosensitizers to lysophospholipids.

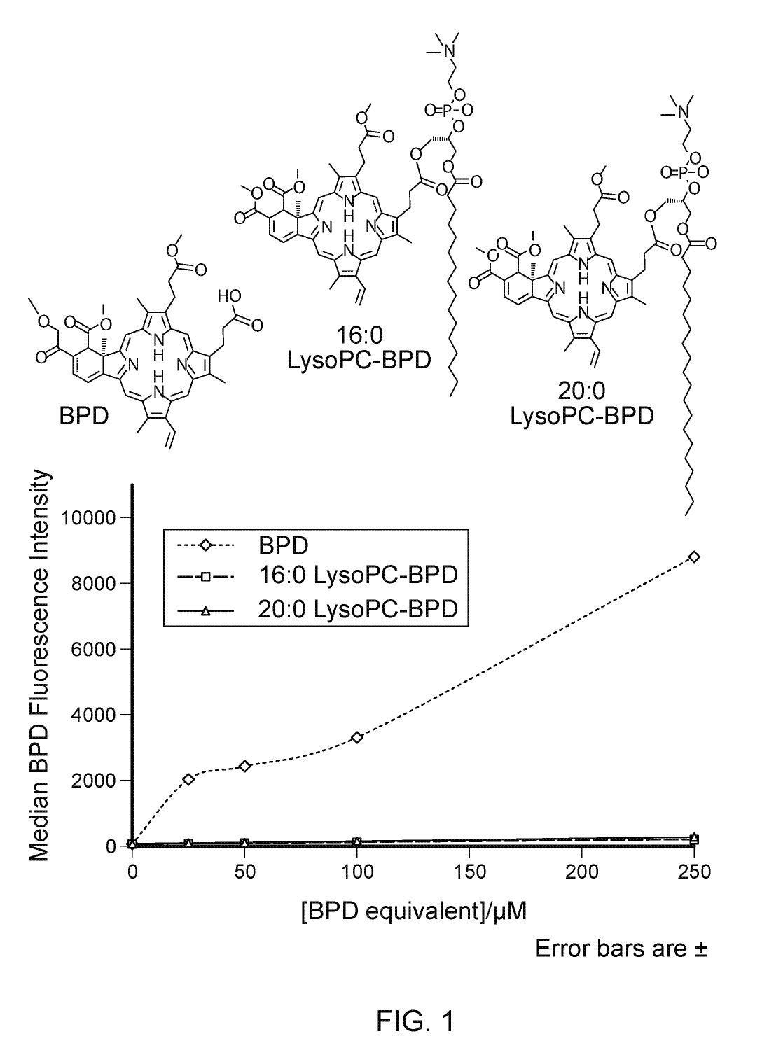

Benzoporphyrin derivative (BPD) conjugates of 16:0 Lyso PC and 20:0 Lyso PC were synthesized and stable liposomes were formed with the 16:0 Lyso PC-BPD and 20:0 Lyso PC-BPD conjugates embedded into the phospholipid bilayer, with the surface fully optimized for click conjugation of azido-modified targeting ligands. To assess non-specific BPD leakage form the liposomes, OVCAR-5 cell suspensions (50,000 cells/condition) were incubated for 30 minutes with varying concentrations of BPD equivalent of liposomes formulated with (1) unconjugated BPD, (2) 16:0 Lyso PC-BPD, and (3) 20:0 Lyso PC-BPD conjugates, then analyzed for BPD updake by flow cytometry. FIG. 1 depicts median BPD fluorescence intensity vs. concentration of BPD in the liposome formulation in OVCAR-5 cells for (1) liposomes with BPD embedded in the lipid bilayer, (2) liposomes with BPD conjugated to 16:0 Lyso PC-BPD, and (3) liposomes with BPD conjugated to 20:0 Lyso PC-BPD. Non-Lyso PC conjugated BPD showed an increase in median fluorescence intensity with increasing concentration of BPD equivalent, indicating non-specific leakage of the BPD into the cells. Lyso PC-conjugated BPD containing lysosomes showed no such leakage, demonstrating that stable liposomes formed with the 16:0 Lyso PC-BPD and 20:0 Lyso PC-BPD block all non-specific leakage of photosensitizers to cells that is observed with hydrophobic BPD entrapment in the bilayer.

### Optimized Surface Functionalization for Click Chemistry

Copper-free click chemistry is possible when the surface of the liposome is derivatized with strongly hydrophobic strained cyclooctyne moieties, such as dibenzocyclooctyne (DIBO).

### PEGylation

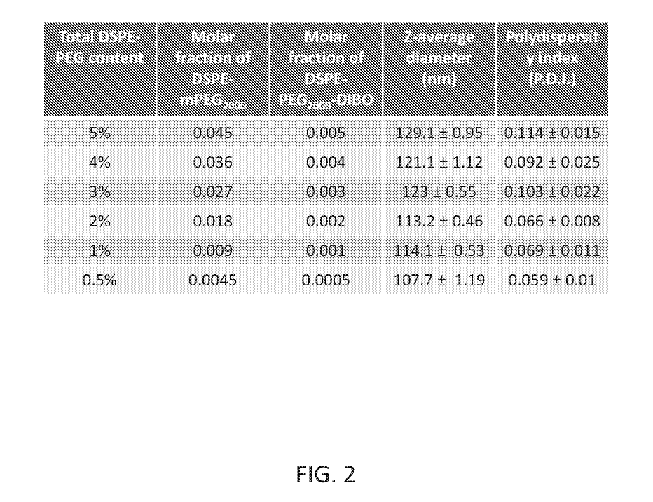

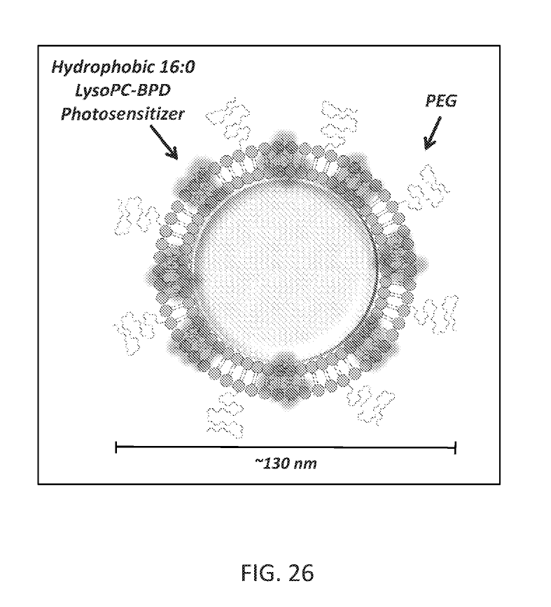

FIG. 2 depicts Z-average diameters and polydispersity indices for liposomes containing 16:0 Lyso PC-BPD, sterically stabilized by the incorporation of 5%, 4%, 3%, 2%, 1% and 0.5% of the phospholipid 1,2-dioleoyl-sn-glycero-3-phosphoethanolamine-N-[methoxy(polyethylene glycol)-2000] (DSPE-mPEG₂₀₀₀). For click chemistry functionality through a strained cyclooctyne (ADIBO)-conjugated lipid 1,2-distearoyl-sn-glycero-3-phosphoethanolamine-N-[dibenzocyclooctyl(polyethylene glycol)-2000] (DSPE-PEG₂₀₀₀-ADIBO), the liposomes remained stable when a 1:10 molar ratio of DSPE-PEG₂₀₀₀-ADIBO:DSPE-mPEG₂₀₀₀ was introduced and maintained. A 9-fold molar counter-stabilization of the hydrophobicity of the DSPE-PEG₂₀₀₀-ADIBO using DSPE-mPEG₂₀₀₀ is believed to support overall liposomal stability. The N-hydroxysuccinimidyl (NHS) ester of ADIBO was coupled with the phospholipid 1,2-distearoyl-sn-glycero-3-phosphoethanolamine-N-[amino(polyethylene glycol)-2000] (DSPE-PEG-NH2). The DSPE-PEG-NH2 was integrated into the liposomes and was counter-stabilized with a 9-fold molar quantity of DSPE-mPEG₂₀₀₀. Copper-free click conjugation of targeting ligands to the stable ADIBO-functionalized liposomes was also performed. FIG. 26 depicts a structural schematic of a liposome including the 16:0 Lyso PC-BPD photosensitizer with PEG chains shown. An example formulation includes 57.6% DPPC, 7.9% DOPG, 28.9% cholesterol, 4.5% DSPE-mPEG₂₀₀₀, 0.5% DSPE-mPEG₂₀₀₀-ADIBO, and 0.6% 16:0 Lyso PC-BPD.

### Electrostatics

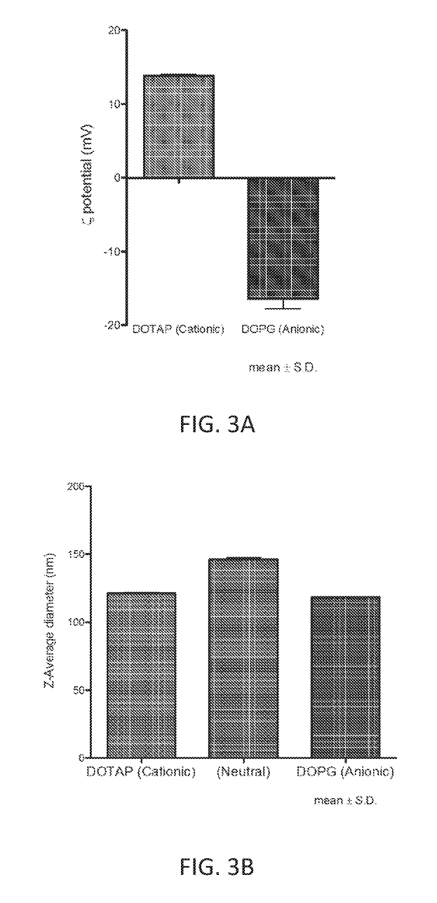

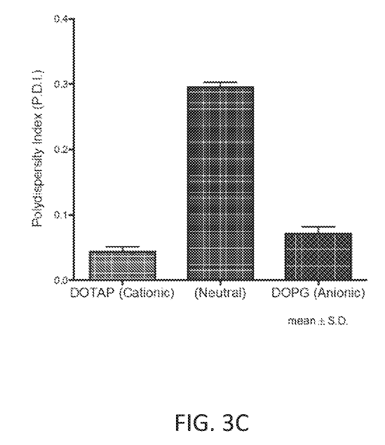

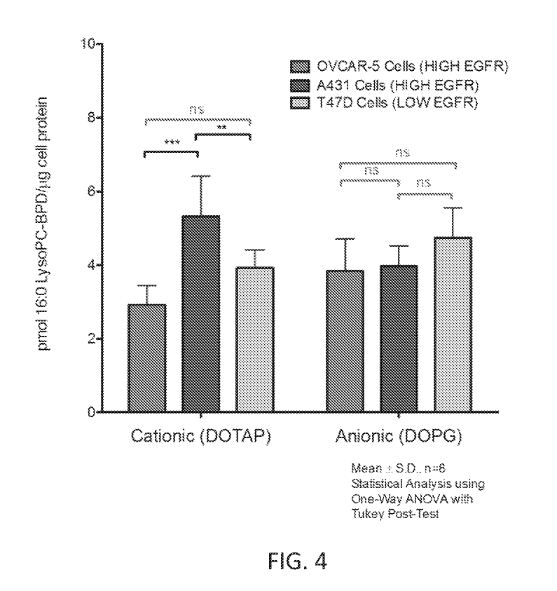

Anionic and cationic charges were introduced into liposomes containing the benzoporphyrin derivative photosensitizer through the incorporation of 7.9% 1,2-dioleoyl-3-trimethylammonium-propane (DOTAP, cationic) or 1,2-dioleoyl-sn-glycero-3-phospho-(1′-rac-glycerol) (DOPG, anionic). FIGS. 3A-3C show ζ-potential, Z average diameter, and polydispersity indices (PDI), respectively, for ADIBO-modified, 16:0 Lyso PC-BPD-containing liposomes containing DOTAP, DOPG, or no added charge. It was found that an anionic or cationic charge provides further electrostatic stabilization of ADIBO-modified, 16:0 Lyso PC-BPD-containing liposomes. The relative polydispersity indices of the charged vs. the neutral liposomes shown in FIG. 3C indicate that without the introduction of charged lipids, the liposomes aggregate and precipitate. FIG. 4 shows the quantitation of cellular update (pmol 16:0 Lyso PC-BPD/μg cell protein) for non-targeted DOTAP- and DOPG-containing liposomes in OVCAR-5 cells, A431 cells, and T47D cells. Non-specific uptake of ADIBO-modified, 16:0 Lyso PC-BPD-containing liposomes was found to be more uniform among the cell lines when the anionic charged lipid DOPG was incorporated into the liposomes. Uniform non-specific uptake profiles promote accurate cellular targeting.

### Phototriggered Release

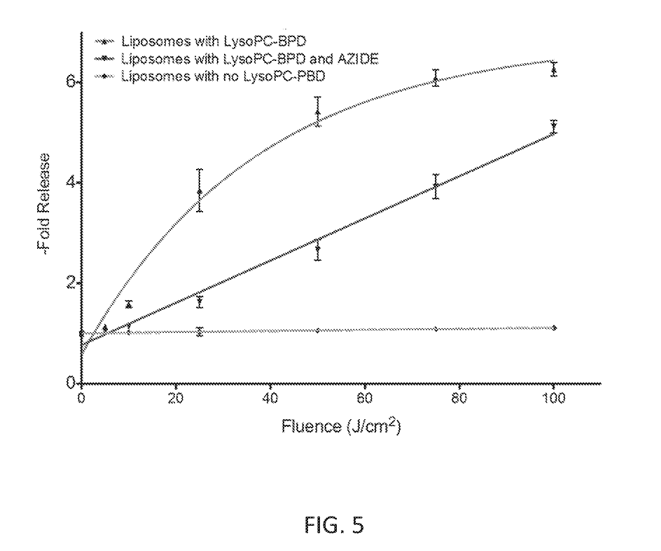

Calcein was loaded within liposomes at concentrations high enough for self-quenching of the fluorophore (100 mM). The liposome formulation either had 16:0 Lyso PC-BPD in the membrane or no photosensitizer as a control. After liposome formation, all extraliposomal calcein fluorophore was purified by dialysis and gel filtration and liposomes were placed into 96 well plates. The liposomes were irradiated with 690 nm light to release intraliposomal calcein which dequenches on release. The dequenching (and increase in fluorescence) of calcein after release was measured using a platereader and normalized to controls prior to irradiation. FIG. 5 shows the fold release of liposomes with (1) Lyso PC-BPD (uppermost plot), (2) Lyso PC-BPD and azide (middle plot), and (3) no Lyso PC-PBD (lowest plot) vs. fluence (light dose) with 690 nm light. Quenched concentrations of the fluorophore calcein disodium salt, a surrogate for liposomally encapsulated therapeutics, was released upon irradiation of liposomes containing 16:0 Lyso PC-BPD in the membrane and surface DSPE-PEG-ADIBO. No release is seen with 16:0 Lyso PC-BPD in the membrane, and release is inhibited in the presence of 10 mM sodium azide. This is believed to show that ADIBO-modified, 16:0 Lyso PC-BPD-containing liposomes are labile to photoxidation and light-triggered release of liposomal payloads. Not wishing to be bound by theory, it is believed that the rate inhibition of phototriggered release in the presence of sodium azide suggests that the process is photochemical and dependent on reactive molecular species.

### Copper-Free Click Conjugation of Targeting Ligands to the Liposomes

### Azido Modification of Antibodies

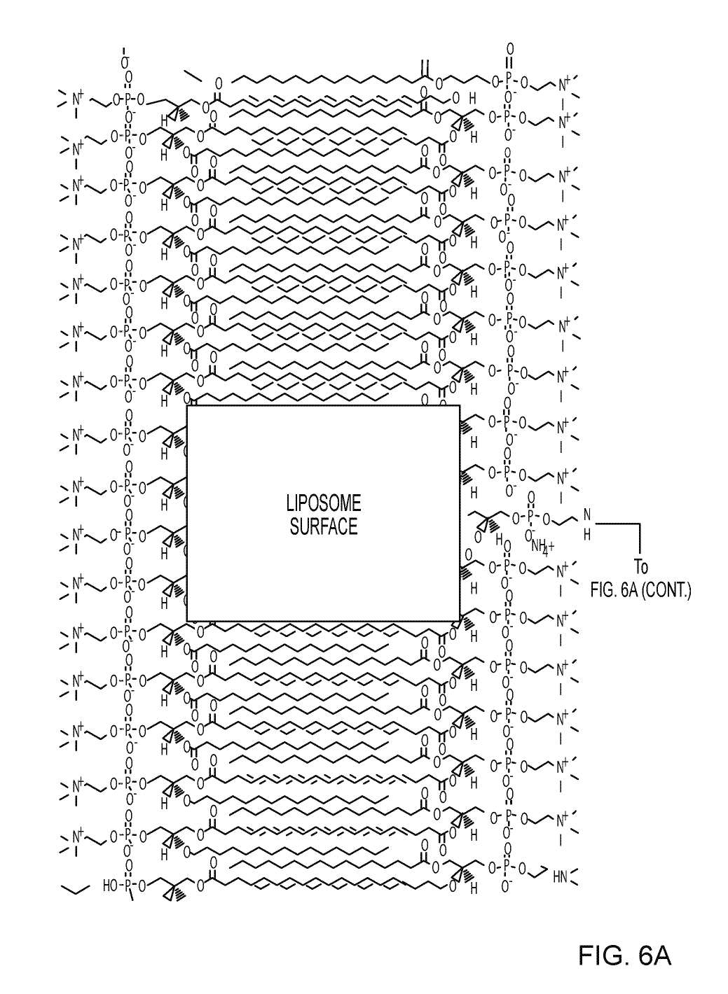

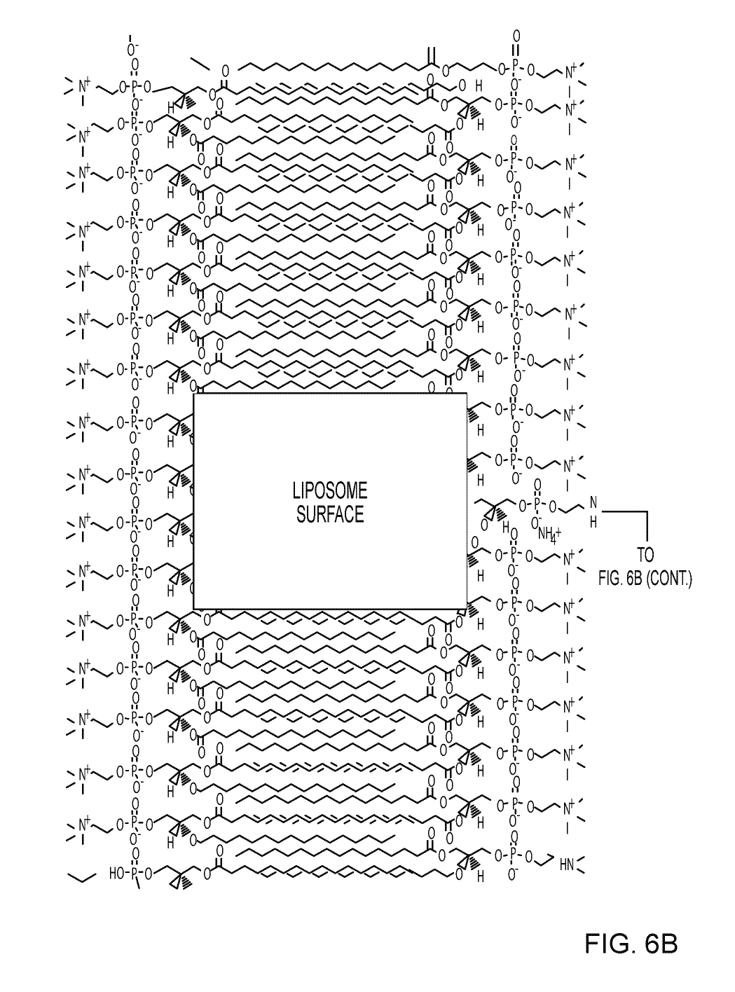

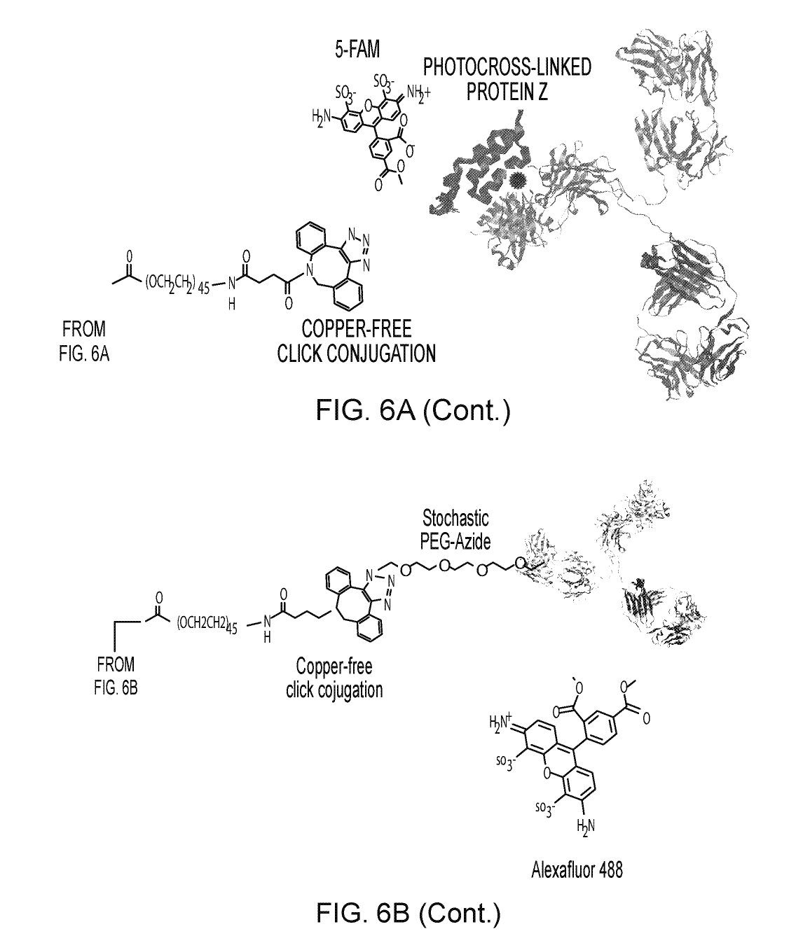

For the copper-free click conjugation of targeting moieties to the liposomal surface that was pre-modified with strained cyclooctynes, the targeting moieties were accessorized with azido groups. Site-specific or stochastic azide modification of Cetuximab (anti-EGFR antibody, Erbitux®) as a proof-of-principle for IgG click conjugation to the optimized liposomal platform containing photosensitizers and/or imaging agents was performed. Site-specific introduction of azide moieties to Cetuximab was performed using a bioengineered Protein Z molecule, which binds only to Fc portions of IgG molecules. (Hui, J. Z., Al Zaki, A., Cheng, Z., Popik, V., Zhang, H., Prak, E. T. L and Tsourkas, A. Facile Method for the Site-Specific, Covalent Attachment of Full-Length IgG onto Nanoparticles (2014) Small 10(16):3354-63. doi: 10.1002/sm11.201303629.) FIG. 6A is a schematic representation of a liposome surface bound to Cetuximab-protein Z through copper-free click chemistry. Protein Z is site-specifically bound to the Fc region of any IgG molecule (e.g., Cetuximab) and is photocross-linked through an unnatural benzoylphenylalanine amino acid. The terminal azide on the peptide-bound protein Z is click conjugated to the optimal surface molar ratio of DSPE-PEG₂₀₀₀-ADIBO, a strained-cyclooctyne derivatized phospholipid. FIG. 6B is a schematic representation of a liposome surface bound to azido-Cetuximab Z through copper-free click chemistry. The terminal azide on the PEG4 molecules stochastically attached to the targeting protein (eg. Cetuximab) is click conjugated to the optimal molar ratio of DSPE-PEG2000-ADIBO, a strained-cyclooctyne derivatized phospholipid.

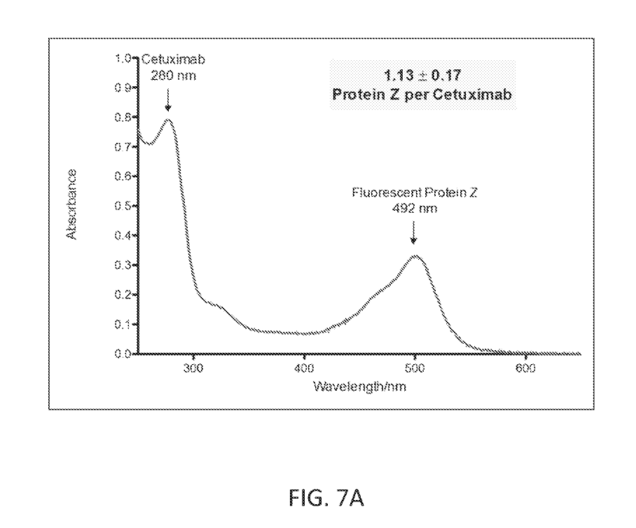

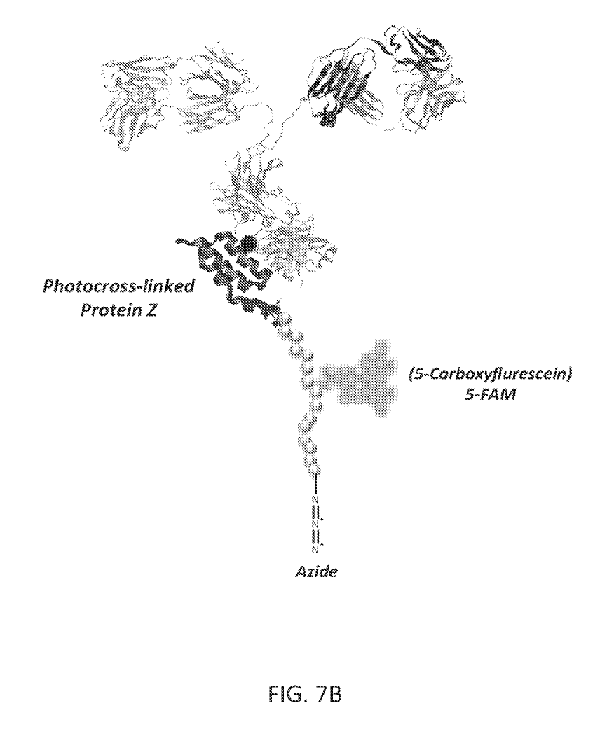

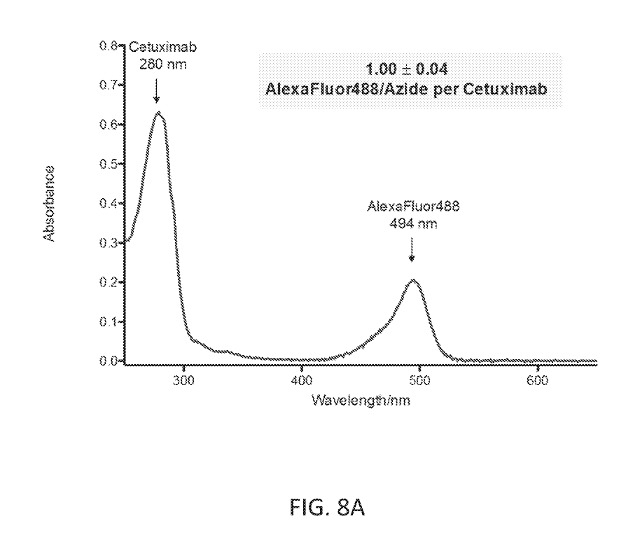

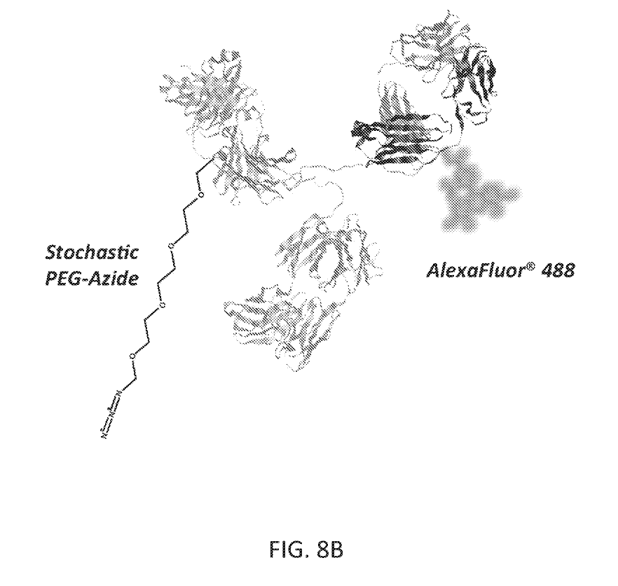

FIGS. 7A-B show the UV-visible spectrum of the site-specific azide conjugate and a structural schematic, respectively. Stochastic introduction of azide moieties to Cetuximab was achieved using an N-hydroxysuccinimidyl ester of PEG4-azide. A single fluorophore (5-FAM for site-specific Cetuximab and AlexaFluor® 488 for stochastic Cetuximab) was introduced into each antibody conjugate. The ratio of protein Z to Cetuximab is 1.13±0.17. FIGS. 8A-B show the UV-visible spectrum of the stochastic azide conjugate incorporating AlexaFluor® 488 and its structural schematic, respectively. The ratio of AlexaFluor® 488 to Cetuximab is 1.00±0.04.

### Click Conjugation

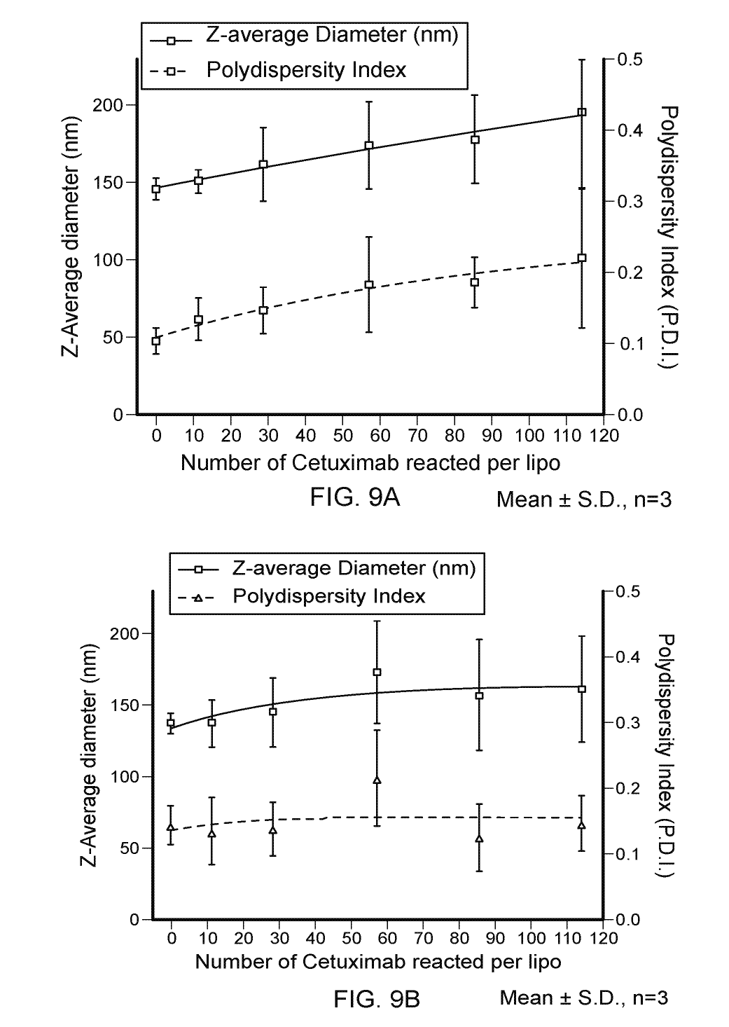

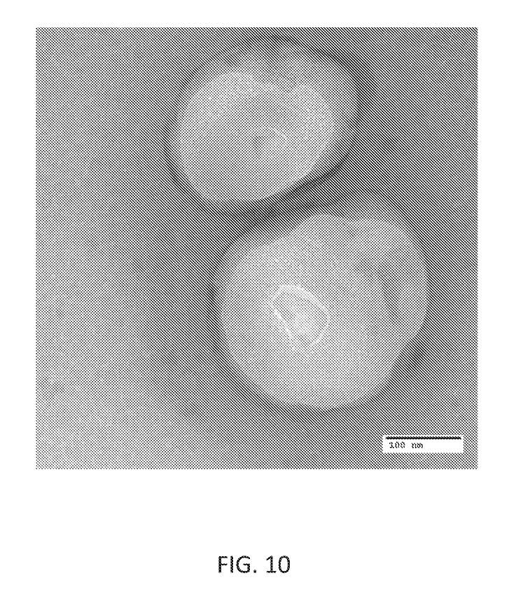

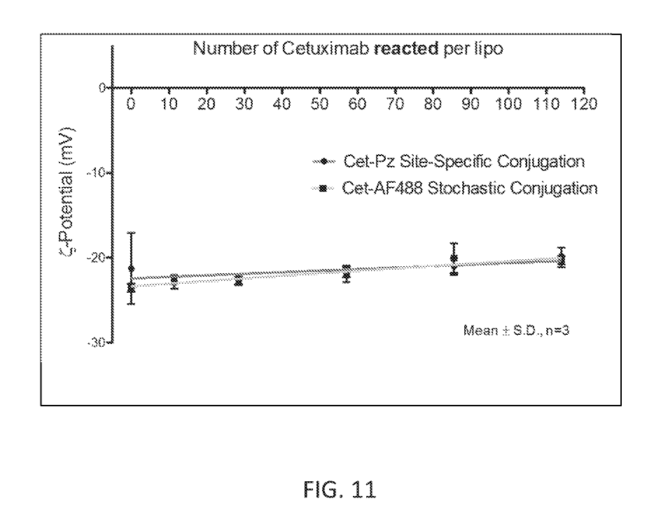

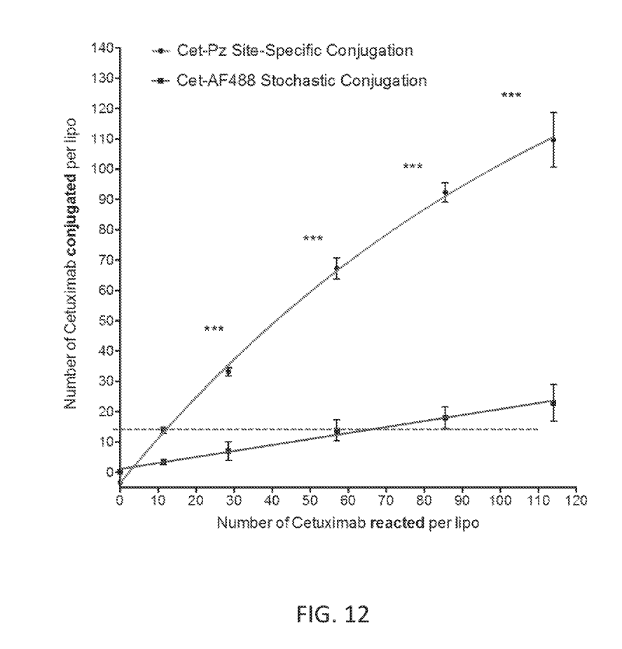

The site-specific and stochastic copper-free click conjugation of the azido modified Cetuximab to ADIBO-modified, 16:0 Lyso PC-BPD-containing anionic liposomes was performed at varying surface densities of conjugated antibodies. Size and dispersity data were collected using dynamic light scattering. FIG. 9 shows comparisons of liposome size (uppermost plots in each figure) and polydispersity index (lower plots in each figure) for site-specific (FIG. 9A) and stochastic (FIG. 9B) conjugation. According to FIGS. 9A and 9B, both site-specific and stochastic conjugation yield stable liposomes. FIG. 10 depicts a transmission electron microscope (TEM) image of liposomes site-specifically click conjugated to Cetuximab-Protein Z imaged at an accelerating voltage of 80 kV and a magnification of 46,000×. FIG. 11 shows comparisons of ζ-potential vs. conjugation efficiency for site-specific (uppermost plot, at left) and stochastic (lower plot, at left) conjugation. FIG. 12 shows comparisons of the number of Cetuximab conjugated per liposome vs. conjugation efficiency for site-specific (uppermost plot) and stochastic (lower plot) conjugation. Based at least on the data shown in FIG. 12, it is believed that that site-specific click conjugation of Cetuximab to liposomes is significantly more efficient than stochastic click conjugation to the liposomes. Not wishing to be bound by theory, it is believed that the lower efficiency observed with stochastic conjugation is an outcome of the lower accessibility of some azide groups to the DIBO moieties, which may be a result of the random introduction of the azide to any part of the antibody.

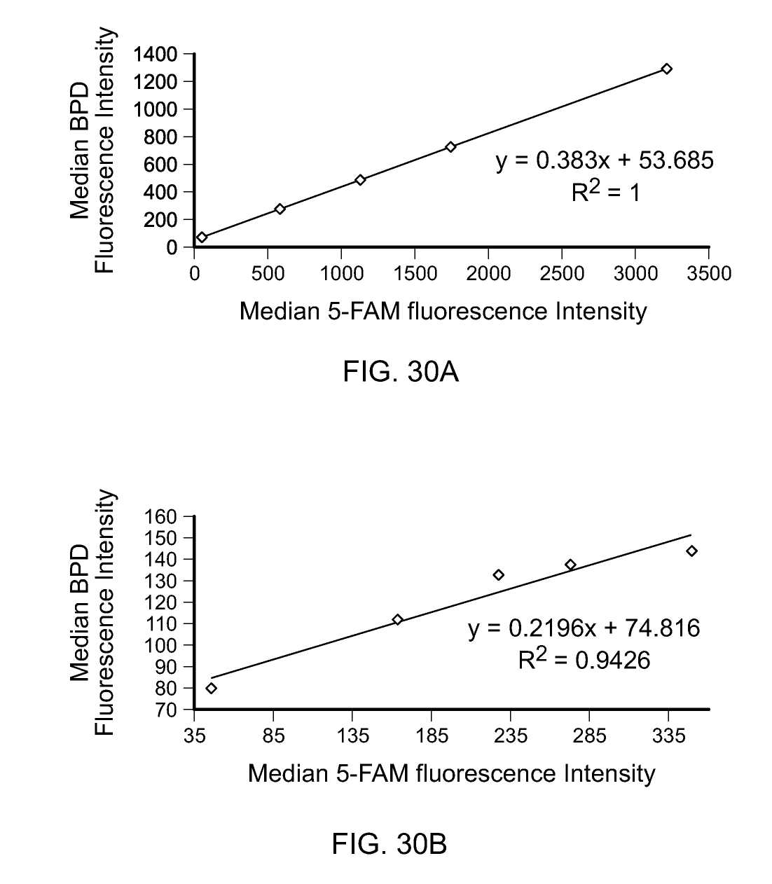

Correlation between the median fluorescence intensity of BPD and the 5-FAM Protein Z bound to Cetuximab in A431 cells is depicted in FIG. 30A (high EGFR) and in T47D cells in FIG. 30B (low EGFR). The high correlation coefficient is indicative of the integrity of the Cetuximab click conjugated to the liposome.

### In Vitro Selectivity of Binding and Phototoxicity

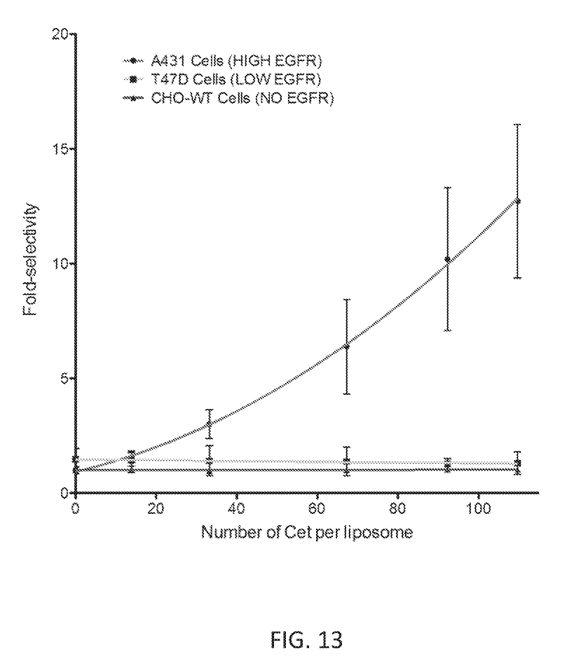

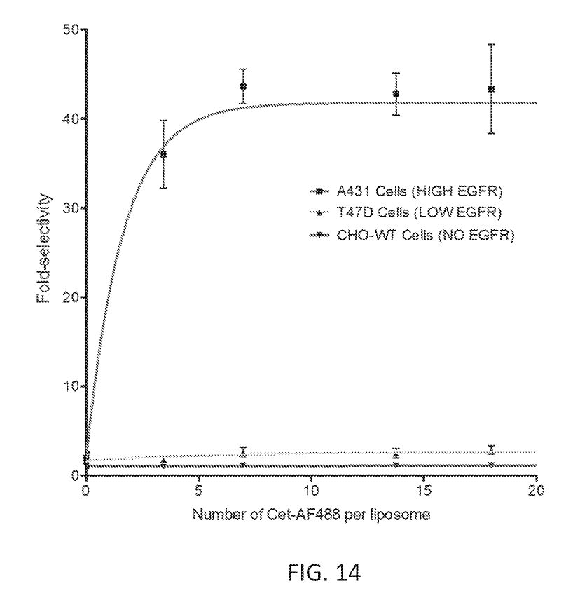

The EGFR selectivity of the targeted liposomes prepared from site-specific or stochastic Cetuximab conjugation at all the densities prepared, was assessed using flow cytometry using BPD fluorescence as a marker for liposome-cell binding. FIG. 13 shows comparisons of binding selectivity in A431 cells (high EGFR; uppermost plot), T47D cells (low EGFR; middle plot) and CHO-WT cells (no EGFR; lower plot). FIG. 14 shows comparisons of binding selectivity of a liposome-Cetuximab conjugate with AlexaFluor® 488 in A431 cells (high EGFR; uppermost plot), T47D cells (low EGFR; middle plot) and CHO-WT cells (no EGFR; lower plot). In FIGS. 13 and 14, selectivity increases with increasing number of Cetuximab per liposome in A431 cells, while exhibiting little change in the T47D and CHO-WT cells. FIG. 14 shows that the selectivity of the stochastic method of conjugation is more efficient at cell binding than the site-specific method.

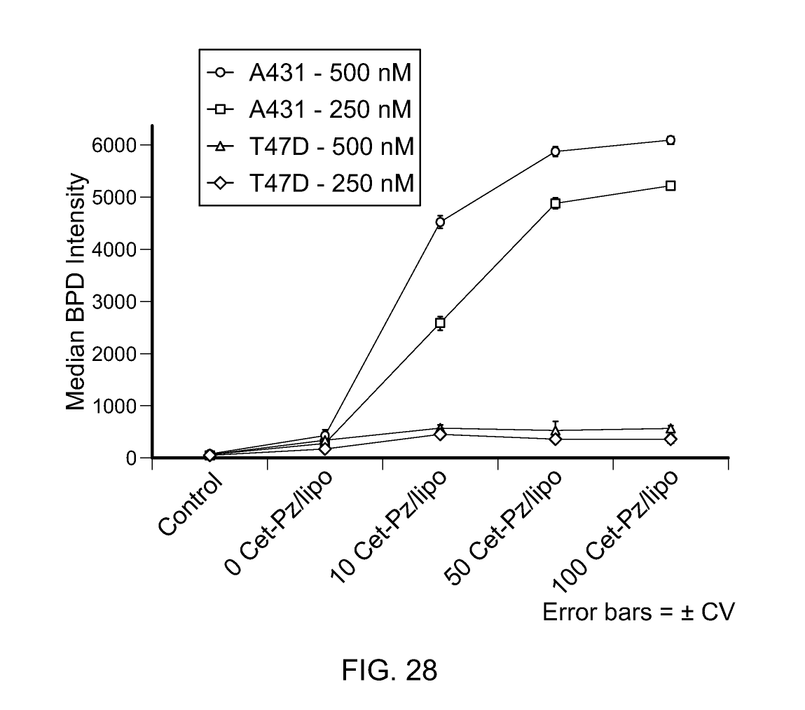

Flow cytometry data is depicted in FIG. 28, and shows the median photosensitizer (BPD) intensity from liposomes reacted with v0, 10, 50 or 100 Cetuximab-Protein Z conjugates (Cet-Pz) per liposome. The liposomes were incubated with either A431 (high EGFR) or T47D (low EGFR) cells at 37° C. for 30 minutes at either 250 nM or 500 nM BPD equivalent of targeted liposome. It is apparent that more reacting 100 Cet-Pz per liposome offers no significant advantage in targeted cell binding over liposomes reacted with 50 Cet-Pz per liposome.

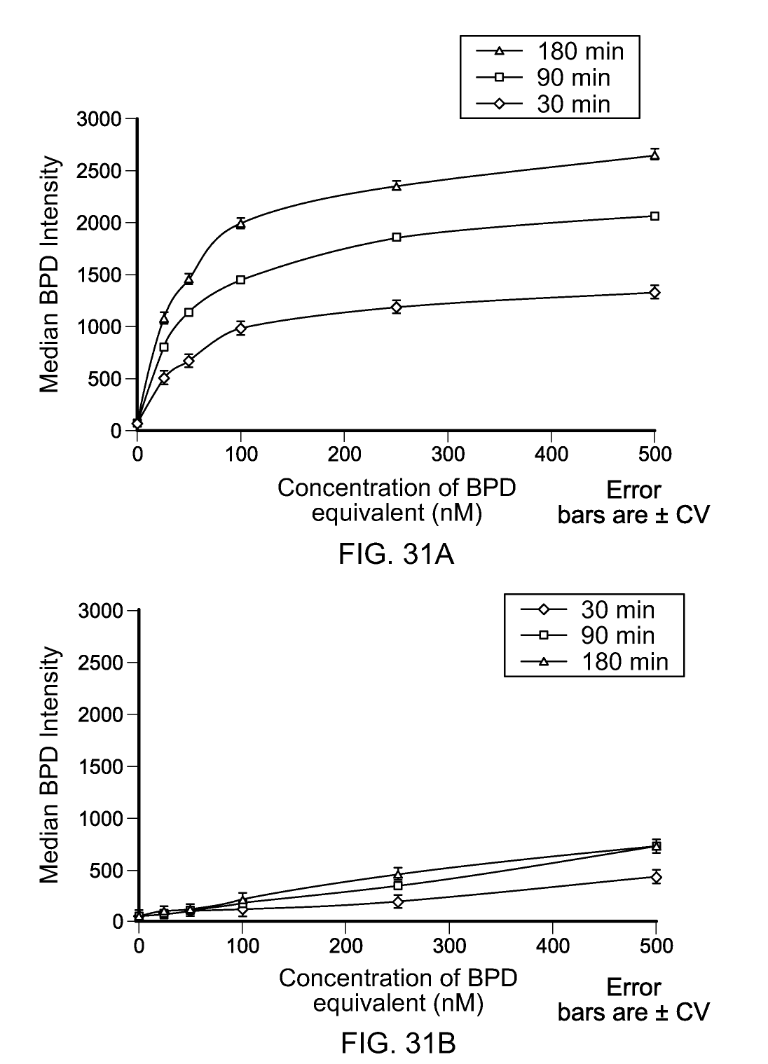

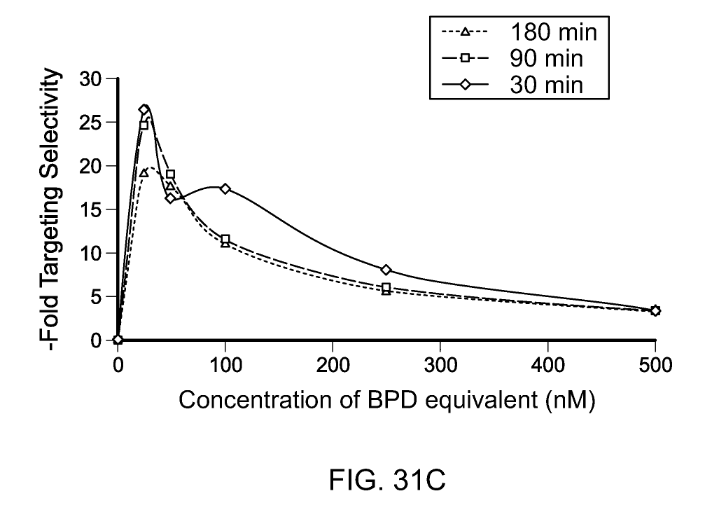

FIGS. 31A and 31B depict flow cytometry data, where the median BPD intensity is the average of 50,000 individually measured cells. The cells were incubated for 30 min, 90 min or 180 minutes with liposomes click conjugated to 50 Cetuximab/liposome (FIG. 31A) versus non-conjugated liposomes (FIG. 31B). The data shows that at all 3 time points, targeted liposomes show preferential binding to OVCAR-5 cells (expressing high EGFR) compared to the non-targeted equivalent. Selectivity of the liposomes (FIG. 31C) is representative of the median BPD intensity of cells incubated with targeted liposomes divided by the median BPD intensity of cells incubated with non-specific liposomes.

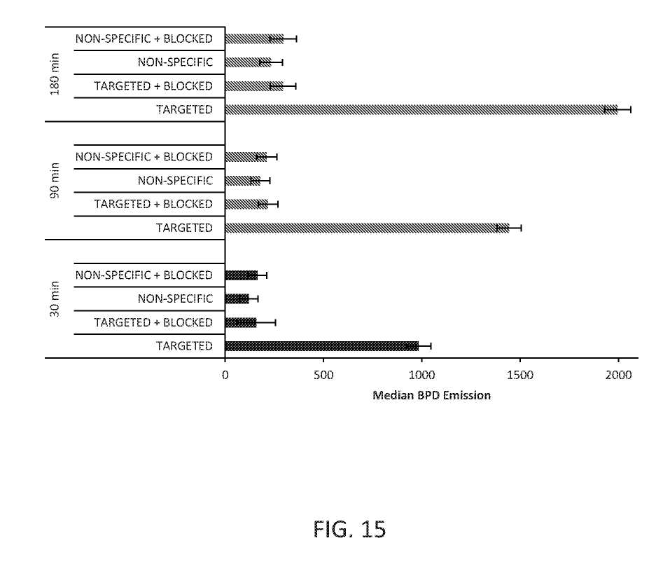

The Cetuximab dependence of the binding of targeted liposomes to the OVCAR-5 cells was validated by incubation with 1 mg/ml free Cetuximab to block the interaction of the targeted liposomes with the cell surface EGFR. FIG. 15 shows flow cytometry data for targeted and non-specific liposomes, as well as targeted and non-specific lyposomes when EGFR was blocked. At all three time points (180 minutes, 90 minutes, and 30 minutes) the presence of free Cetuximab completely blocked the binding of the targeted liposomes to OVCAR-5 cells, as explained by the reduction in the median BPD emission signal that resulted from targeted 16:0 Lyso PC-BPD liposome binding to the cells.

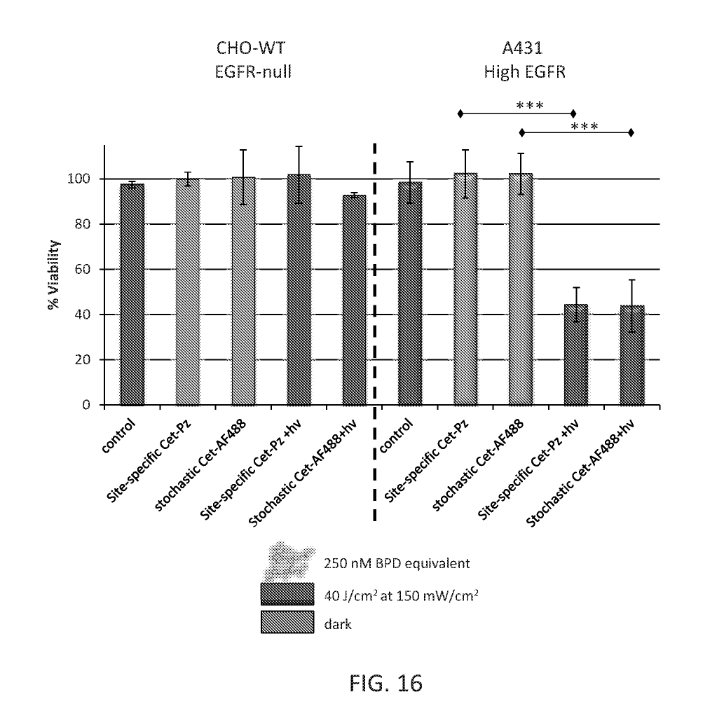

Selectivity of phototoxicity was compared in CHO-WT cells (no EGFR) and A431 cells (high EGFR) using 690 nm laser irradiation. FIG. 16 shows the cell viabilities following selective photodynamic therapy using 16:0 Lyso PC-BPD liposomes conjugated site-specifically and stochastically to Cetuximab. Without irradiation, incubation with either targeted liposomes has no effect on viability on either A431 cells or CHO-WT cells. The targeted liposomes induced negligible levels of phototoxicity in the CHO-WT cells, whereas A431 viability was reduced to approximately 50%, underscoring the capacity for the targeted liposomal platform to mediate selective cancer therapies. Metabolic activity, indicative of viability was assessed 24 hours following PDT treatment using the MTT colorimetric assay (*=P≤0.05, **=P≤0.01, ***=P≤0.001).

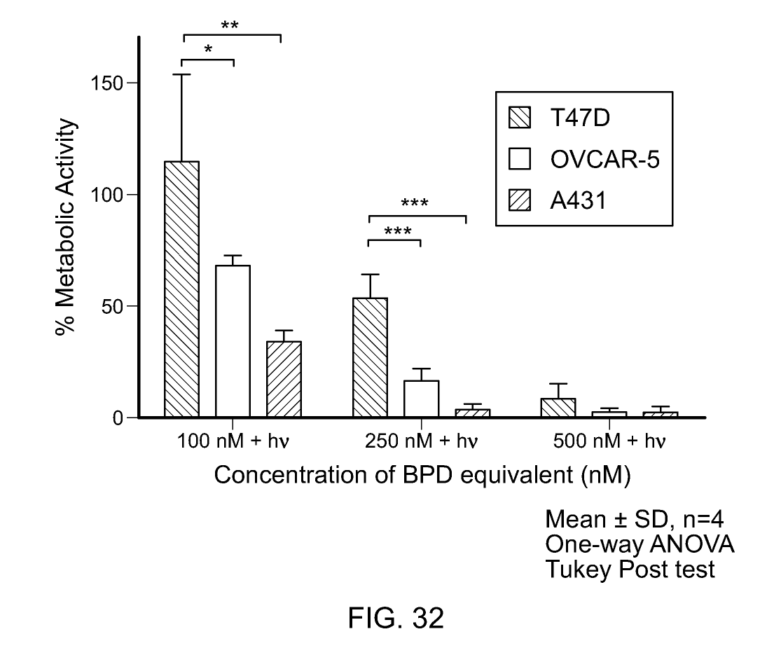

FIG. 32 depicts the results of an MTT assay of A431 and OVCAR-5 cells (high EGFR) and T47D cells (low EGFR) following PDT treatment with 20 J/cm² of 690 nm laser light. The stochastically targeted liposomes were incubated for 24 hours with the cells. Metabolic activity, indicative of cell viability was assessed 24 hours following PDT treatment using the MTS colorimetric assay (*=P≤0.05, **=P≤0.01, ***=P≤0.001). Cell viability of T47D cells was highest at all BPD concentrations, and cell viability was lower with increasing BPD concentration.

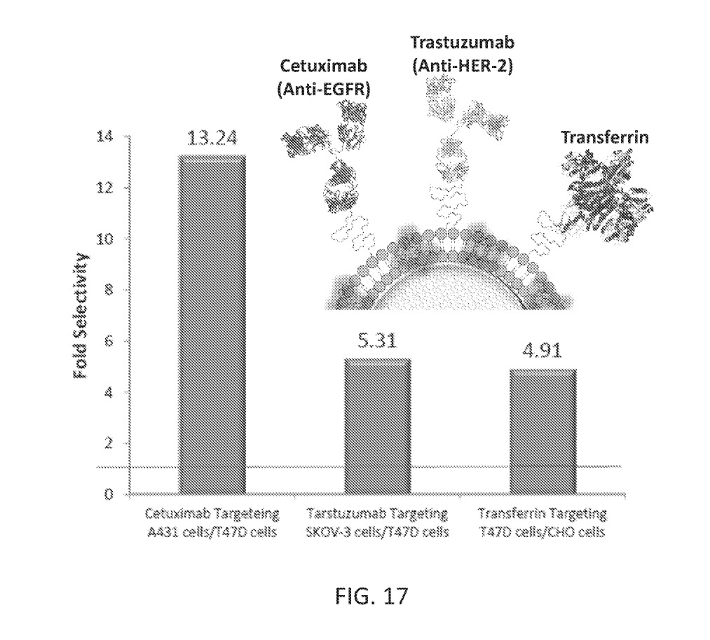

NHS-PEG₄-N₃ can also be stochastically conjugated to any primary amine-containing targeting ligand and can be click conjugated to the same photosensitizing nanoconstruct. This was demonstrated by the conjugation of Cetuximab (anti-EGFR antibody), Trastuzumab (anti-HER-2 antibody) and human transferrin (natural ligand for transferrin receptor). Fold selectivity was obtained using flow cytometry and is representative of the median BPD fluorescence of cells overexpressing the target receptor divided by the median BPD signal of the cells expressing little or no target receptor (FIG. 17). The red line represents 1-fold selectivity where there is no preferential binding of the targeted liposome to the target cells over the non-target cells. A431 cells are human epidermoid carcinoma cells overexpressing the EGFR receptor, SKOV-3 cells are ovarian carcinoma cells overexpressing the HER-2 receptor, T47D cells are breast pleural effusion carcinoma cells overexpressing the transferrin receptor and CHO cells are non-cancerous Chinese Hamster Ovary cells. FIG. 17 demonstrates that stochastic azido modification of Trastuzumab (anti-HER-2 antibody, Herceptin®) and transferrin (natural ligand of transferrin receptor) exhibit cellular selectivity of binding when clicked to ADIBO-modified, 16:0 Lyso PC-BPD-containing liposomes.

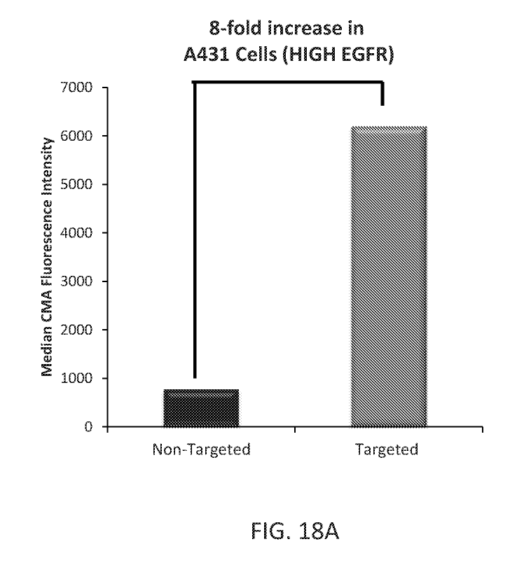

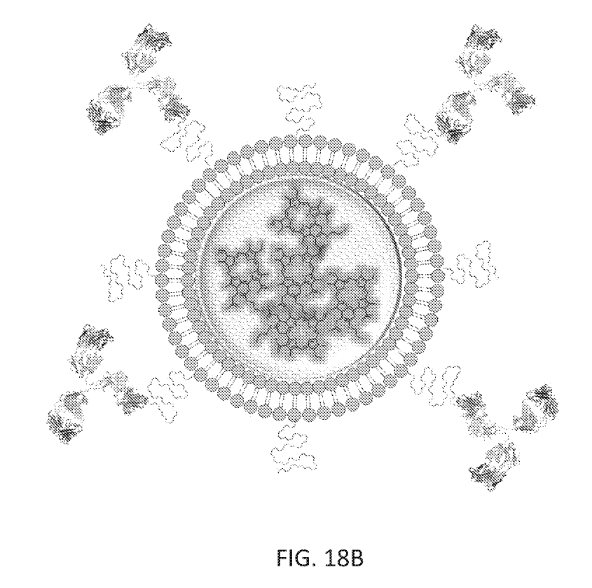

ADIBO-modified liposomes with no membrane entrapped 16:0 Lyso PC-BPD were loaded with the water-soluble photosensitizer chlorin e6 monoethylene diamine monoamide (CMA) and shown to offer cellular selectivity when stochastically conjugated to Cetuximab (FIG. 18A). A schematic of the liposome is shown in FIG. 18B.

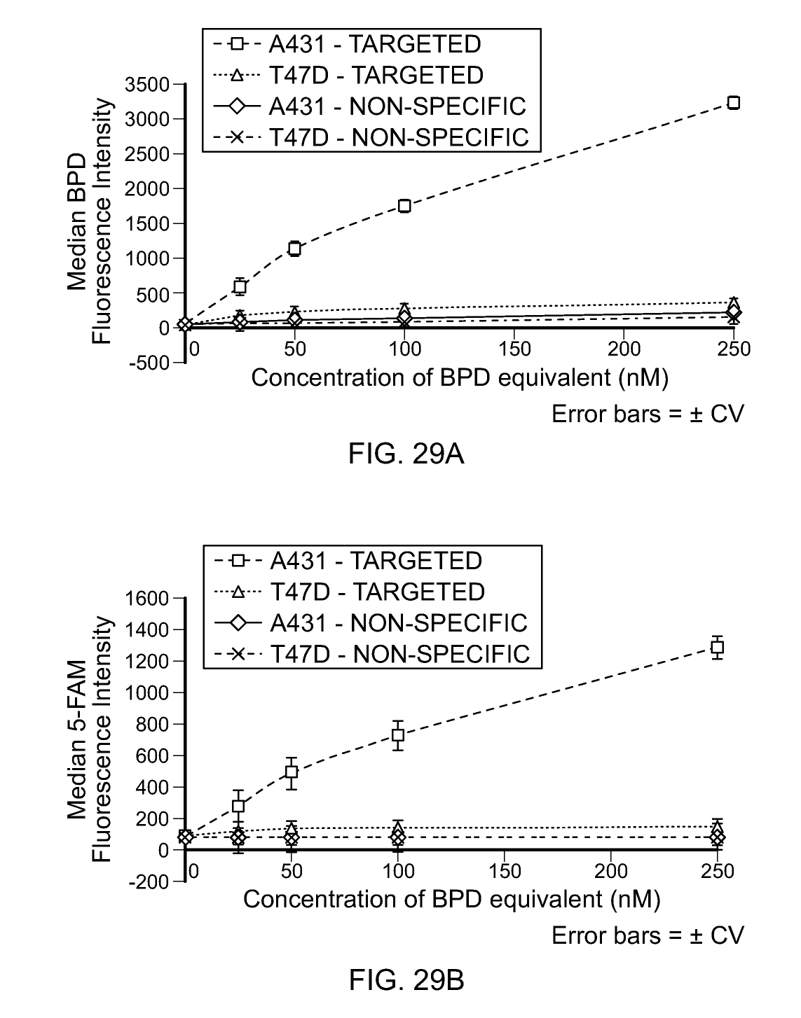

Flow cytometry data was obtained depicting the median photosensitizer (BPD) intensity originating from the liposome membrane (FIG. 29A) and the median 5-FAM fluorescence intensity originating from the fluorescently labeled peptide attached to Protein Z (FIG. 29B). A431 (high EGFR) and T47D (low EGFR) cells were incubated at 37° C. for 30 minutes with 25, 50, 100 and 250 nM BPD equivalent of targeted Cetuximab-Protein Z clicked liposomes or non-specific liposomes without antibody conjugation. This data shows that there is a strong positive correlation between the EGFR-dependent cellular binding of the fluorescently labeled Cetuximab-Protein Z and 16:0 Lyso PC-BPD embedded in the membrane of the click conjugated liposomes. The correlation validates that copper-free click conjugated liposomes are a stable entity that can uniformly deliver liposomal payloads to target cancer cells.

### Selective Endocytosis:

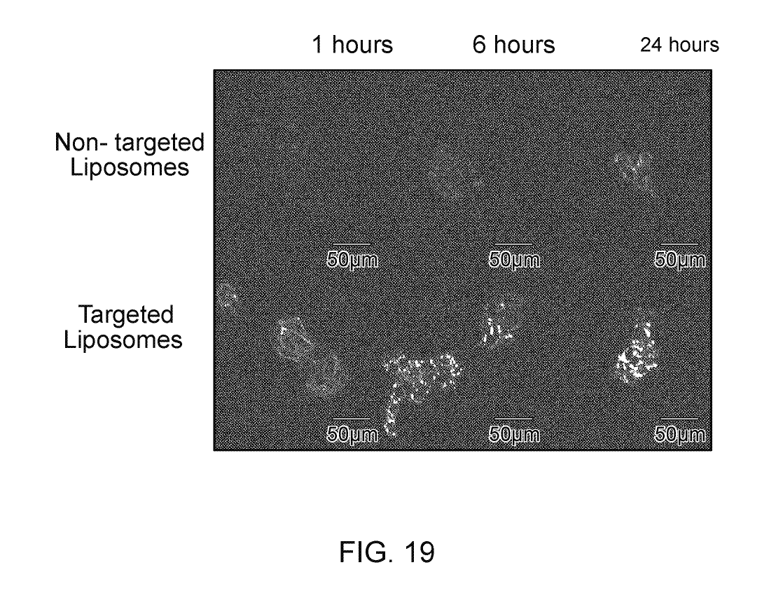

The selective intracellular delivery of the targeted liposomal platform enables intracellular induction of photodynamic therapy and intracellular phototriggered delivery of biological/chemotherapeutic agents. FIG. 19 shows confocal fluorescence microscopy images of non-targeted and stochastically targeted liposomes in OVCAR-5 cells at progressive time intervals, indicating cellular internalization of the targeted liposomes. The micrographs demonstrate that 16:0 Lyso PC-BPD containing liposomes click conjugated to Cetuximab can be selectively delivered intracellularly within OVCAR-5 cells that overexpress EGFR in a time-dependent manner.

### Fluorophore Labeling of Liposomal Nanoconstructs for In Vitro Imaging and In Vivo Bioimaging of Targeted Nanoconstructs.

Stable anchoring of hydrophilic fluorophores can be achieved through their conjugation (1) to cholesterol, (2) to the phosphate head group of phospholipids like 1,2-dipalmitoyl-sn-glycero-3-phosphoethanolamine (DPPE) or (3) to the terminal end of PEGylated phospholipids such as DSPE-PEG₂₀₀₀-NH2.

### Modular Fluorophore Labeling

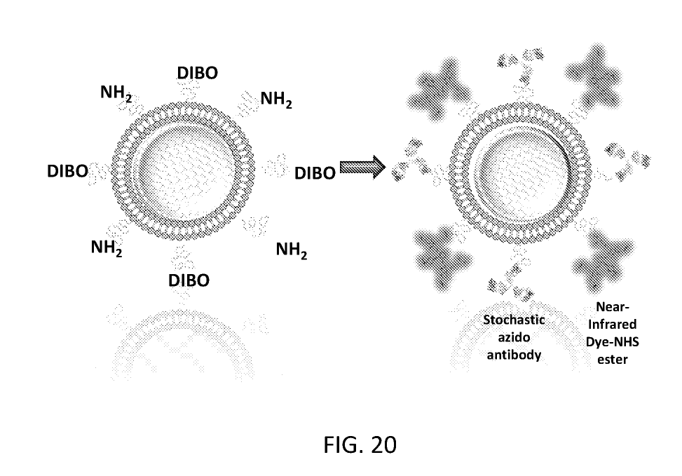

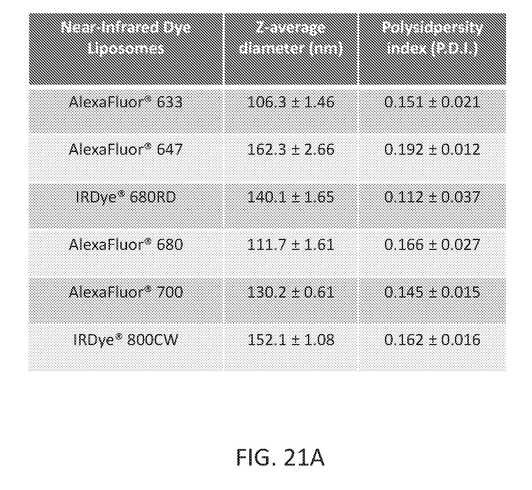

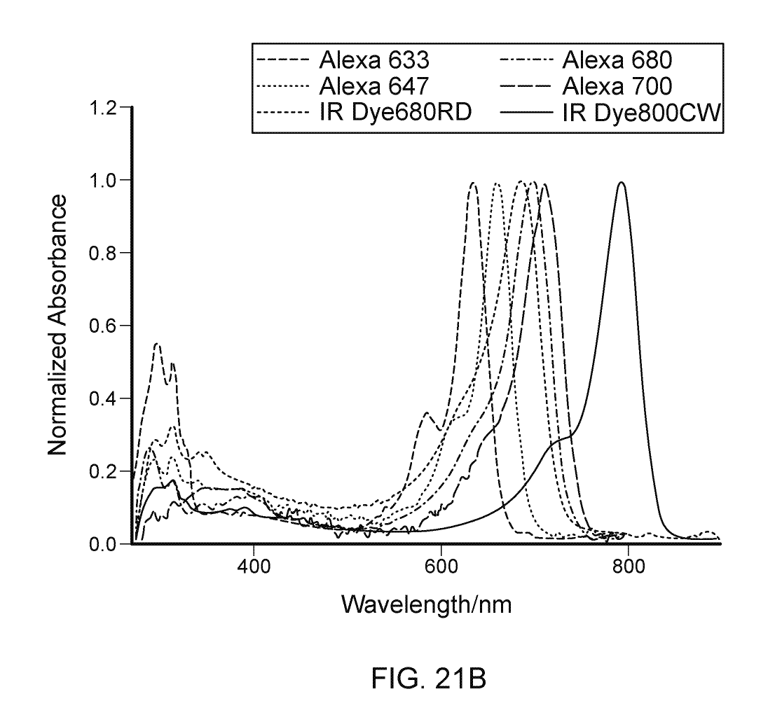

The high chemoselectivity of ADIBO as a component of copper-free click chemistry enabled the modular conjugation of different reactive entities to the surface of the liposomes without cross-reactivity with the ADIBO. 0.2% DSPE-PEG₂₀₀₀-NH2 was incorporated within ADIBO-modified liposomes and conjugated with amine-reactive NHS-esters of the near-infrared dyes to prepare a panel of stable click-reactive liposomes capable of use in in vivo imaging (FIG. 20). The dyes used to label ADIBO-modified liposomes include AlexaFluor® 633, AlexaFluor® 647, AlexaFluor® 680, AlexaFluor®700, IRDye® 680RD and IRDye® 800CW. Formulations prepared included 58.2% DPPC, 7.9% DOPG/DOTAP, 28.9% cholesterol, 4.3% DSPE-mPEG₂₀₀₀, 0.5% DSPE-PEG₂₀₀₀-ADIBO (for click conjugation to antibodies), and 0.2% DSPE-PEG₂₀₀₀-NH2 (for amide coupling to dyes). All the liposomes remained stable after they were click conjugated stochastically to Cetuximab or a sham IgG1 control. FIG. 21A shows size and polydispersity indices of liposomes that include the various dyes, and FIG. 21B shows UV-visible spectra of each.

### In Vitro Selectivity of Fluorophore-Labeled Liposomes

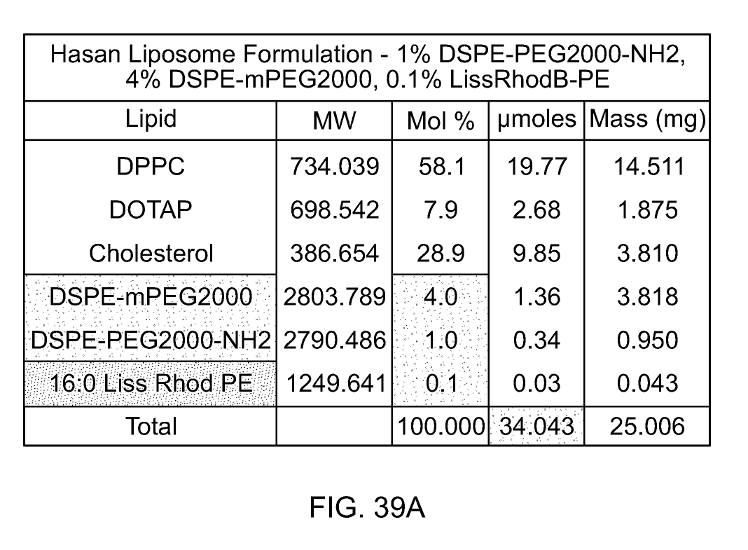

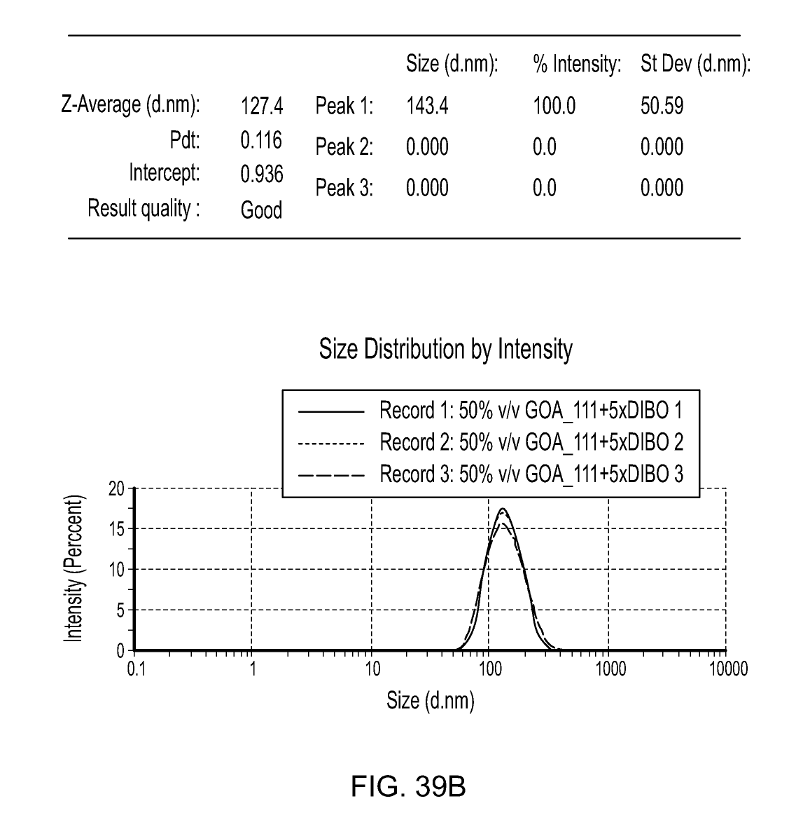

An example formulation incorporating a lipid anchored fluorophore Lissamine Rhodamine B-DPPE is shown in FIG. 39A. To incorporate the ADIBO, 5 equivalents of dibenzocyclooctyne NHS ester (ADIBO-NHS 0.426 mM) was reacted with the amines in pH 8 100 mM phosphate buffer with 10% DMSO. Size and polydispersity indices for three samples (50% v/v GOA_111+5×ADIBO 1 (uppermost plot), 50% v/v GOA_111+5×ADIBO 2 (middle plot), and 50% v/v GOA_111+5×ADIBO 3 (lowest plot) are shown in FIG. 39B.

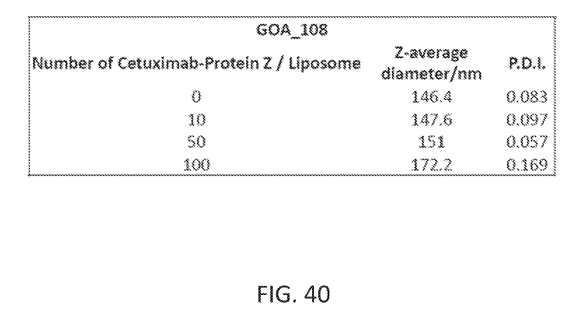

Liposomes including Rhodamine were incubated with 0, 10, 50, and 100 Cetuximab-Protein Z per liposome overnight at room temperature in 50 mL aliquots. Each conjugate was purified from unbound antibody using Sepharose CL-4B gel filtration chromatography and characterized using UV-visible spectroscopy and dynamic light scattering (DLS). FIG. 40 shows size and polydispersity indices for each sample.

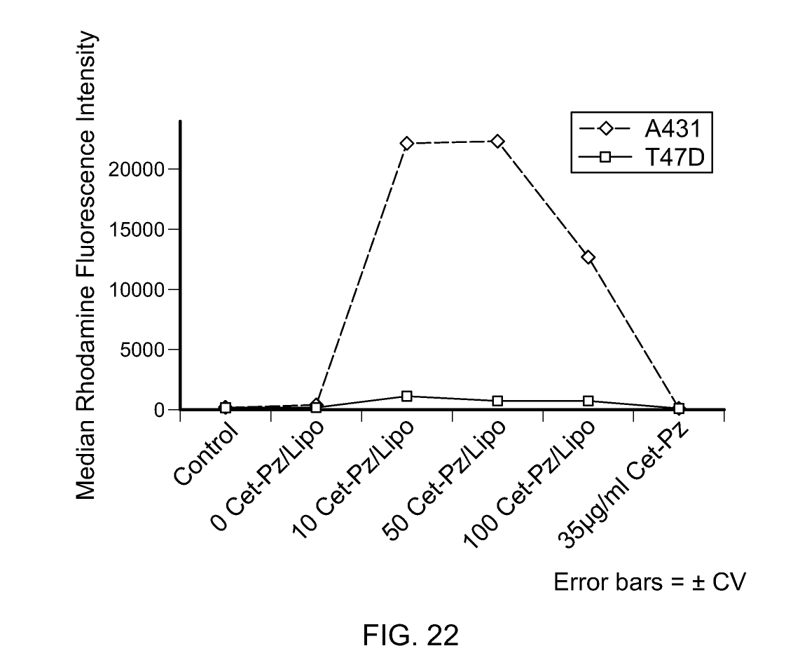

0.1% of DPPE bound to Lissamine Rhodamine B was incorporated within ADIBO-modified liposomes and click conjugated site-specifically to Cetuximab. FIG. 22 shows median Lissamine Rhodamine B intensity as a function of the Cetuximab-Protein Z (Cet-Pz) density per liposome, demonstrating that the fluorescently labeled liposomes selectively bind to A431 cells (high EGFR; upper plot) compared to T47D cells (low EGFR; lower plot). Optimal densities occur between 10 to 50 Cet-Pz/liposome.

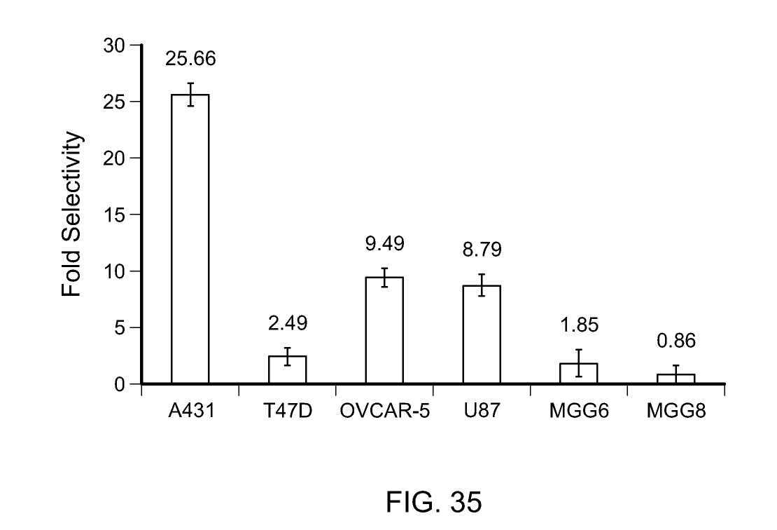

FIG. 35 shows fold selectivity for a variety of cancer cell lines using GOA_111-CetPz (10 CetPz/liposome) median Lissamine Rhodamine B intensity, at 500 nM Lissamine Rhodamine B equivalent. 100 μL liposome samples were incubated for 30 minutes with 50,000 cells at 37° C. The high EGFR A431 cells exhibit the highest selectivity.

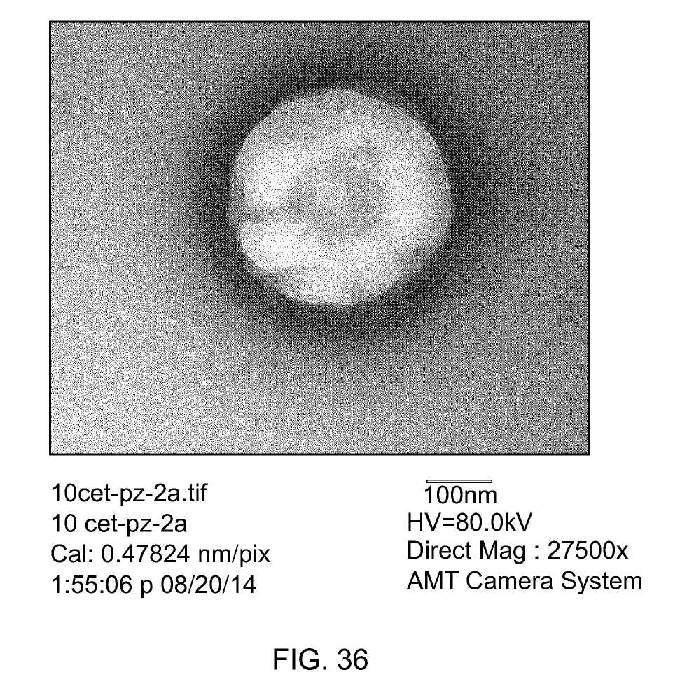

A BPD-Rhodamine liposome conjugated to Cetuximab-Protein Z was prepared and imaged by transmission electron microscopy. 200 μl GOA_113 with 0 or 10 Cetuximab per liposome was prepared, then 2000×g 100 kDa ultrafiltered for 20 minutes. 10 μl of concentrated liposomes were loaded onto copper mesh carbon coated grid for 1 minute, followed by staining with phosphotungstic acid for 10 seconds. The samples were dried overnight. FIG. 36 shows the transmission electron microscope (TEM) image of a liposome processed according to the foregoing procedure.

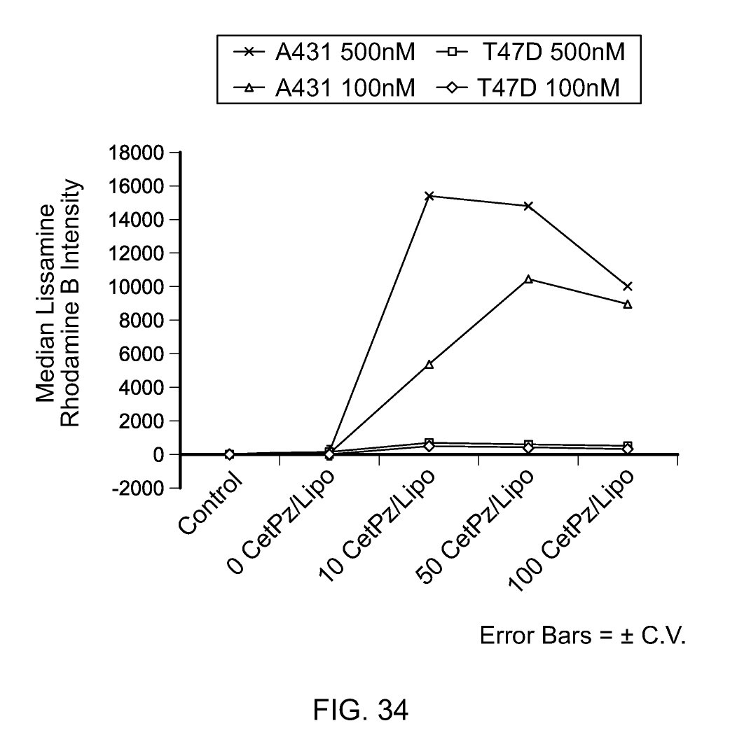

FIG. 34 shows optimal Cetuximab-Protein Z density for A431 cells at 500 nM and 100 nM concentrations of Lissamine Rhodamine B (uppermost plot and next plot down, respectively) and for T47D cells at 500 nM and 100 nM concentrations of Lissamine Rhodamine B (lower two plots). Optimal density is 10 CetPz/liposome at 500 nM in A431, and 50 CetPz/liposome at 100 nM in A431.

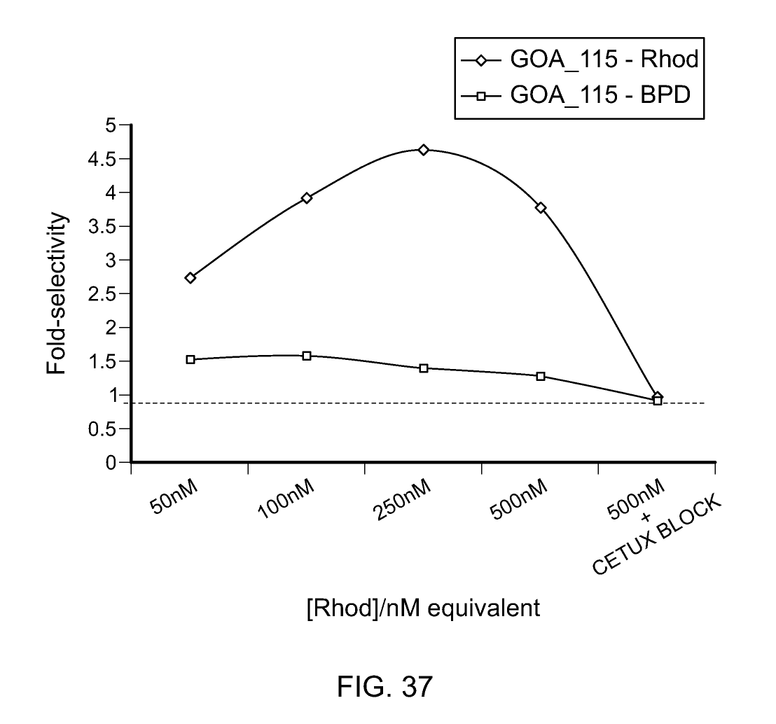

FIG. 37 shows fold selectivity vs. nM equivalent of Rhodamine, showing that liposomes that are click conjugated to Cetuximab and include both a lipid anchored Rhodamine dye and hydrophobically entrapped free BPD show differential levels of selectivity compared to liposomes without antibody conjugation. Because the lipid anchored Rhodamine is fixed in the membrane that is covalently conjugated to the antibody, up to 4.5-fold selectivity in Rhodamine binding (as determined by FACS fluorescence signal) is seen at 30 minutes in a concentration-dependent manner. Free Cetuximab blocks this selectivity, confirming Rhodamine (and liposome) selectivity is dependent on EGFR binding. The free BPD hydrophobically entrapped in the same liposomes has very different selectivity profiles where barely 1.5-fold preference is observed in the targeted liposomes over the non-targeted liposomes. Based on these observations, antibody conjugation provides liposome selectivity, but free BPD leeches out into the cells irrespective of liposome binding.

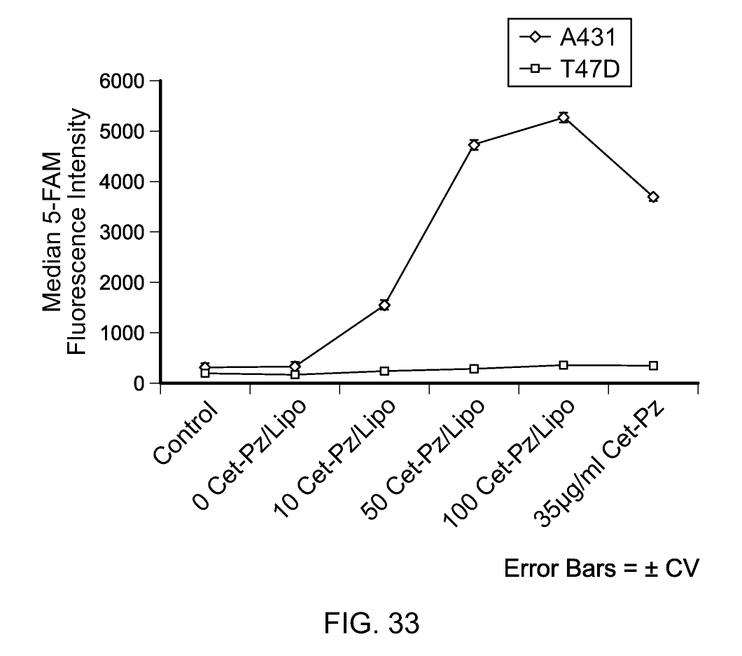

FIG. 33 shows median 5-FAM fluorescence intensity as a function of Cetuximab-Protein Z density per liposome, also demonstrating high selectivity for A431 cells (upper plot) compared to T47D cells (lower plot). Optimal density occurs at about 100 Cet-Pz/liposome.

### In Vivo Bioimaging and Selectivity

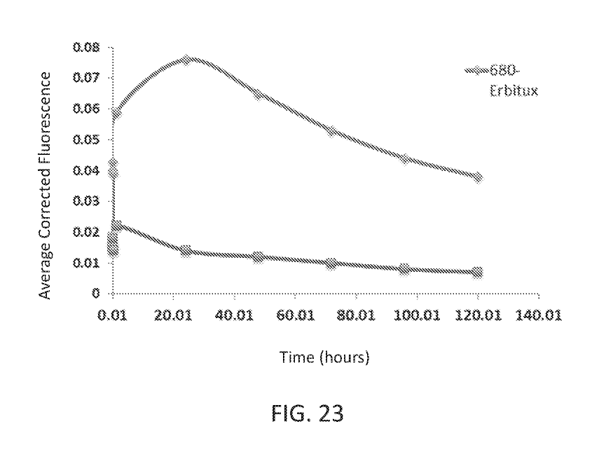

ADIBO-modified liposomes labeled with IRDye® 680RD and IRDye® 800CW can be stochastically click conjugated to Cetuximab or human IgG1 sham antibody control, respectively. FIG. 23 shows the average corrected fluorescence of IRDye® 680RD (upper plot) and IRDye® 800CW (lower plot) vs. time, demonstrating selective binding of the IRDye680RD labeled liposome click conjugated to Cetuximab (Erbitux) to subcutaneous U251 tumors, compared to co-injected IRDye800CW labeled liposome click conjugated to a sham human IgG1 control.

Subcutaneous U251 murine xenograft glioblastoma tumors overexpressing EGFR were imaged following intravenous injection of 0.1 nmol dye equivalent of each liposome into mice (targeted liposomes click conjugated to 50 stochastic PEG-azide Cetuximab molecules per liposome and 50 stochastic PEG-azide non-specific IgG molecules per liposome). The targeted liposomes were tagged with the near-infrared fluorophore IRDye® 680RD and the non-specific liposomes were tagged with the near-infrared fluorophore IRDye® 800CW. A dual tracer imaging approach using the PEARL imaging system was performed. The targeted liposomes were found to reach maximal selectivity at 24 hours after administration, as compared to the IgG1 sham conjugated liposomes.

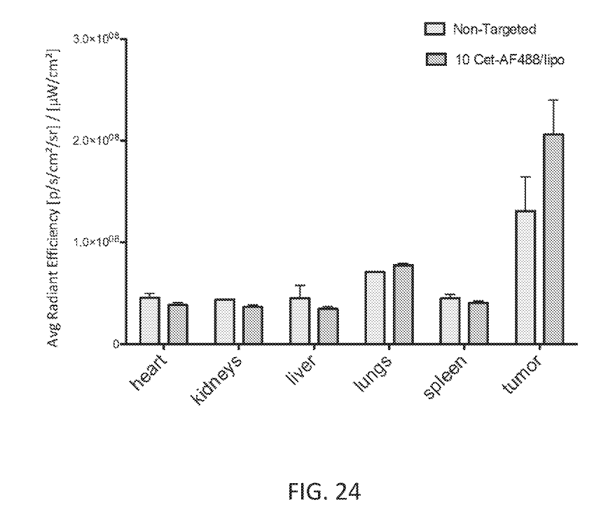

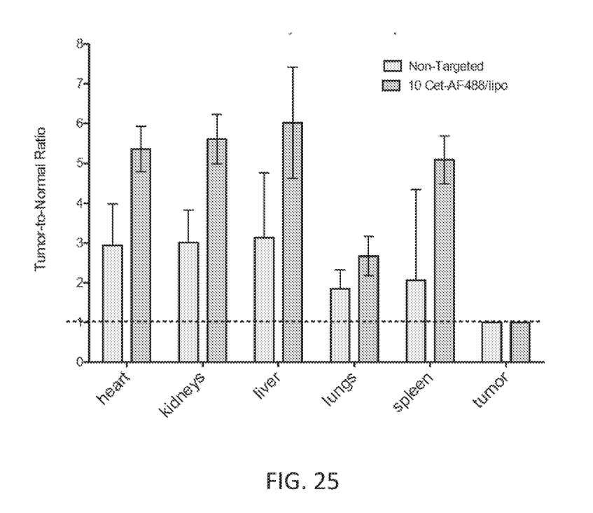

In a separate biodistribution study of targeted phototherapeutic 16:0 Lyso PC-BPD containing liposomes, mice bearing subcutaneous A431 epidermoid carcinoma tumors were injected with 0.25 mg/kg BPD equivalent of non-targeted and stochastic Cetuximab targeted liposomes. The tumors and organs were harvested at 24 hours following injection and the 16:0 Lyso PC-BPD biodistribution was quantitatively imaged. FIG. 24 shows that the targeted liposome (right bar in each pair) was more selective for the A431 tumor than the non-targeted liposome (left bar in each pair of bars). FIG. 25 shows that the fluorescence of 16:0 Lyso PC-BPD can be used to quantitatively image the biodistribution and confirm the tumor selectivity of stochastically click conjugated liposomes in mice with subcutaneous A431 (high EGFR) tumors 24 hours after administration of 0.25 mg/kg BPD equivalent, compared to non-conjugated DIBO-containing 16:0 Lyso PC-BPD liposome controls.

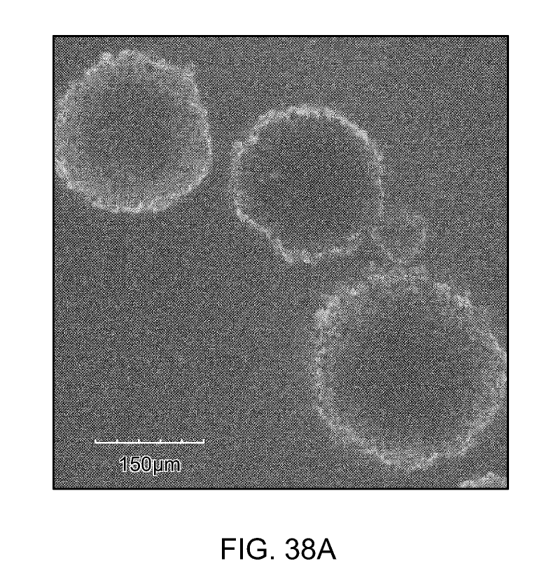

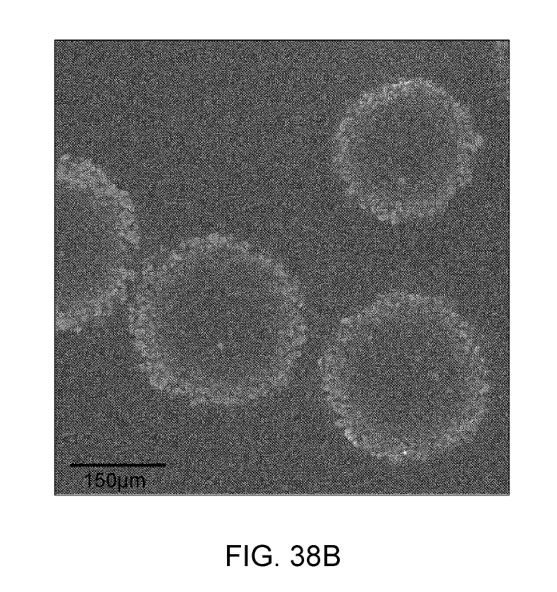

FIG. 38 shows confocal fluorescence microscope images of MGG6 cell neurospheres (nuclei stained with Hoechst 33324, blue) incubated with Rhodamine labelled liposomes (red) for 30 minutes. FIG. 38 shows that at 30 minutes, targeted liposomes that are click conjugated to Cetuximab-anti EGFR show preferential binding (FIG. 38A), compared to non-targeted liposomes (FIG. 38B).

A number of embodiments of the invention have been described. Nevertheless, it will be understood that various modifications may be made without departing from the spirit and scope of the invention. Accordingly, other embodiments are within the scope of the following claims.

1. A liposome comprising:
a) a conjugate comprising a lysophospholipid and a photosensitizer;
b) a first derivatized phospholipid comprising a first phospholipid and a strained cyclooctyne moiety;
c) a second derivatized phospholipid comprising a second phospholipid and a polyethylene glycol polymer; and
d) a cationic or anionic lipid.

2. A liposome comprising:
a) a conjugate comprising a lysophospholipid and a photosensitizer;
b) a first derivatized phospholipid comprising a first phospholipid and a targeting moiety;
c) a second derivatized phospholipid comprising a second phospholipid and a polyethylene glycol polymer; and
d) a cationic or anionic lipid.

3.-10. (canceled)

11. The liposome of claim 1, wherein the conjugate comprising a lysophospholipid and a photosensitizer is present at about 0.01 mol percent to about 1 mole percent in the liposome.

12.-14. (canceled)

15. The liposome of claim 1, wherein the first phospholipid is selected from the group consisting of: hydrogenated soy phosphotidylcholine (HSPC); 1,2-didecanoyl-sn-glycero-3-phosphocholine (DDPC); 1,2-dierucoyl-sn-glycero-3-phosphate (DEPA); 1,2-dielaidoyl-sn-glycero-3-phosphocholine (DEPC); 1,2-dierucoyl-sn-glycero-3-phosphoethanolamine (DEPE); 1, 2-dielaidoyl-phosphatidylglycerol (DEPG); 1,2-dilinoleoyl-sn-glycero-3-phosphocholine (DLOPC); 1,2-dilauroyl-sn-glycero-3-phosphate (DLPA); 1,2-dilauroyl-sn-glycero-3-phosphocholine (DLPC); 1,2-dilauroyl-sn-glycero-3-phosphoethanolamine (DLPE); 1,2-dilauroyl-sn-glycero-3-phosphatidylglycerol (DLPG); 1,2-dilauroyl-sn-glycero-3-phosphoserine (DLPS); 1,2-dimyristoyl-sn-glycero-3-phosphate (DMPA); 1,2-dimyristoyl-sn-glycero-3-phosphocholine (DMPC); 1,2-dimyristoyl-sn-glycero-3-phosphoethanolamine (DMPE); 1,2-dimyristoyl-sn-glycero-3-phosphatidylglycerol (DMPG); 1,2-dimyristoyl-sn-glycero-3-phosphoserine (DMPS); 1,2-dioleoyl-sn-glycero-3-phosphate (DOPA); 1,2-dioleoyl-sn-glycero-3-phosphocholine (DOPC); 1,2-dioleoyl-sn-glycero-3-phosphoethanolamine (DOPE); 1,2-dioleoyl-sn-glycero-3-phosphatidylglycerol (DOPG); 1,2-dioleoyl-sn-glycero-3-phosphoserine (DOPS); 1,2-dipalmitoyl-sn-glycero-3-phosphate (DPPA); 1,2-dipalmitoyl-sn-glycero-3-phosphocholine (DPPC); 1,2-dipalmitoyl-sn-glycero-3-phosphoethanolamine (DPPE); 1,2-dipalmitoyl-sn-glycero-3-phosphatidylglycerol (DPPG); 1,2-dipalmitoyl-sn-glycero-3-phosphoserine (DPPS); 1,2-distearoyl-sn-glycero-3-phosphate (DSPA); 1,2-distearoyl-sn-glycero-3-phosphocholine (DSPC); 1,2-distearoyl-sn-glycero-3-phosphoethanolamine (DSPE); 1,2-distearoyl-sn-glycero-3-phosphatidylglycerol (DSPG); 1,2-distearoyl-sn-glycero-3-phosphoserine (DSPS); 1-myristoyl-2-palmitoyl-sn-glycero 3-phosphocholine (MPPC); 1-myristoyl-2-stearoyl-sn-glycero-3-phosphocholine (MSPC); 1-palmitoyl-2-myristoyl-sn-glycero-3-phosphocholine (PMPC); 1-palmitoyl-2-oleoyl-sn-glycero-3-phosphocholine (POPC); 1-palmitoyl-2-oleoyl-sn-glycero-3-phosphoethanolamine (POPE); 1-palmitoyl-2-oleoyl-sn-glycero-3-phosphatidylglycerol (POPG); 1-palmitoyl-2-stearoyl-sn-glycero-3-phosphocholine (PSPC); 1-stearoyl-2-myristoyl-sn-glycero-3-phosphocholine (SMPC); and 1-stearoyl-2-oleoyl-sn-glycero-3-phosphocholine (SOPC); 1-stearoyl-2-palmitoyl-sn-glycero-3-phosphocholine (SPPC), and combinations thereof; and
wherein the strained cyclooctyne comprises an aza-dibenzocyclooctyne (ADIBO), dibenzocyclooctyne (DIBO), (OCT), aryl-less octyne (ALO), monofluorinated cyclooctyne (MOFO), difluorinated cyclooctyne (DIFO), biarylazacyclooctynone (BARAC), or a dimethoxyazacyclooctyne (DIMAC) moiety.

16. (canceled)

17. The liposome of claim 15, wherein the first derivatized phospholipid further comprises a linker.

18. The liposome of claim 15, wherein the linker is a polyethylene glycol having a molecular weight selected from the group consisting of 350 g/mol, 550 g/mol, 750 g/mol, 1000 g/mol, 2000 g/mol, 3000 g/mol, 5000 g/mol, 10,000 g/mol, 20,000 g/mol and 30,000 g/mol, and
wherein the first derivatized phospholipid comprises DSPE-PEG₂₀₀₀.

19.-24. (canceled)

25. The liposome of claim 2, wherein the targeting moiety is selected from the group consisting of: a folate, a RGD peptide, a natural protein ligand, an affibody, an antibody, an antibody fragment, and an engineered antibody-based protein.

26. (canceled)

27. The liposome of claim 2, wherein the targeting moiety is conjugated to the first derivatized phospholipid using Cu-free click chemistry.

28. The liposome of claim 2, wherein the targeting moiety comprises an azide moiety prior to conjugating with the first phospholipid.

29. The liposome of claim 28, wherein the first derivatized phospholipid comprises the first phospholipid and the reaction product of a strained cyclooctyne and the targeting moiety comprising an azide.

30.-31. (canceled)

32. The liposome of claim 1, wherein the second phospholipid is selected from the group consisting of: hydrogenated soy phosphotidylcholine (HSPC); 1,2-didecanoyl-sn-glycero-3-phosphocholine (DDPC); 1,2-dierucoyl-sn-glycero-3-phosphate (DEPA); 1,2-dielaidoyl-sn-glycero-3-phosphocholine (DEPC); 1,2-dierucoyl-sn-glycero-3-phosphoethanolamine (DEPE); 1, 2-dielaidoyl-phosphatidylglycerol (DEPG); 1,2-dilinoleoyl-sn-glycero-3-phosphocholine (DLOPC); 1,2-dilauroyl-sn-glycero-3-phosphate (DLPA); 1,2-dilauroyl-sn-glycero-3-phosphocholine (DLPC); 1,2-dilauroyl-sn-glycero-3-phosphoethanolamine (DLPE); 1,2-dilauroyl-sn-glycero-3-phosphatidylglycerol (DLPG); 1,2-dilauroyl-sn-glycero-3-phosphoserine (DLPS); 1,2-dimyristoyl-sn-glycero-3-phosphate (DMPA); 1,2-dimyristoyl-sn-glycero-3-phosphocholine (DMPC); 1,2-dimyristoyl-sn-glycero-3-phosphoethanolamine (DMPE); 1,2-dimyristoyl-sn-glycero-3-phosphatidylglycerol (DMPG); 1,2-dimyristoyl-sn-glycero-3-phosphoserine (DMPS); 1,2-dioleoyl-sn-glycero-3-phosphate (DOPA); 1,2-dioleoyl-sn-glycero-3-phosphocholine (DOPC); 1,2-dioleoyl-sn-glycero-3-phosphoethanolamine (DOPE); 1,2-dioleoyl-sn-glycero-3-phosphatidylglycerol (DOPG); 1,2-dioleoyl-sn-glycero-3-phosphoserine (DOPS); 1,2-dipalmitoyl-sn-glycero-3-phosphate (DPPA); 1,2-dipalmitoyl-sn-glycero-3-phosphocholine (DPPC); 1,2-dipalmitoyl-sn-glycero-3-phosphoethanolamine (DPPE); 1,2-dipalmitoyl-sn-glycero-3-phosphatidylglycerol (DPPG); 1,2-dipalmitoyl-sn-glycero-3-phosphoserine (DPPS); 1,2-distearoyl-sn-glycero-3-phosphate (DSPA); 1,2-distearoyl-sn-glycero-3-phosphocholine (DSPC); 1,2-distearoyl-sn-glycero-3-phosphoethanolamine (DSPE); 1,2-distearoyl-sn-glycero-3-phosphatidylglycerol (DSPG); 1,2-distearoyl-sn-glycero-3-phosphoserine (DSPS); 1-myristoyl-2-palmitoyl-sn-glycero 3-phosphocholine (MPPC); 1-myristoyl-2-stearoyl-sn-glycero-3-phosphocholine (MSPC); 1-palmitoyl-2-myristoyl-sn-glycero-3-phosphocholine (PMPC); 1-palmitoyl-2-oleoyl-sn-glycero-3-phosphocholine (POPC); 1-palmitoyl-2-oleoyl-sn-glycero-3-phosphoethanolamine (POPE); 1-palmitoyl-2-oleoyl-sn-glycero-3-phosphatidylglycerol (POPG); 1-palmitoyl-2-stearoyl-sn-glycero-3-phosphocholine (PSPC); 1-stearoyl-2-myristoyl-sn-glycero-3-phosphocholine (SMPC); and 1-stearoyl-2-oleoyl-sn-glycero-3-phosphocholine (SOPC); 1-stearoyl-2-palmitoyl-sn-glycero-3-phosphocholine (SPPC); and combinations thereof.

33.-36. (canceled)

37. The liposome of claim 1, wherein the polyethylene glycol polymer is terminated with an alkoxyl, carboxyl, amine, biotin, maleimide, succinyl, N-hydroxysuccinimidyl ester, sulfo-N-hydroxysuccinimidyl ester, silane, pyridyldithiol or cyanur moiety.

38.-41. (canceled)

42. The liposome of claim 1, wherein the liposome comprises an anionic lipid.

43. (canceled)

44. The liposome of claim 42, wherein the anionic lipid is selected from the group consisting of: 1,2-dioleoyl-sn-glycero-3-phospho-(1′-rac-glycerol) (DOPG); phosphatidyl glycerol (PG); 1,2-dimyristoyl-sn-glycero-3-phospho-(1′-rac-glycerol) (DMPG); phosphatidylserine (PS); 1,2-dioleoyl-sn-glycero-3-phospho-L-serine (DOPS); and dicetylphosphate (DCP).

45.-46. (canceled)

47. The liposome of claim 1, wherein the liposome comprises a cationic lipid.

48.-51. (canceled)

52. The liposome of claim 1, wherein the liposome further comprises a cargo selected from the group consisting of one or more therapeutic compounds; one or more polymeric nanoparticles; one or more protein-based nanoparticles; one or more dendrimeric structures; one or more chitosan nanoparticle; one or more polyglycan structures; one or more inorganic nanoparticles; one or more imaging agents; one or more kinase inhibitors; one or more biologics; and combinations of two or more thereof;
wherein the one or more kinase inhibitors is a receptor tyrosine kinase inhibitor.

53. (canceled)

54. The liposome of claim 52, wherein one or more of the one or more therapeutic compounds exhibit synergy with the photosensitizer.

55. The liposome of claim 1, wherein the liposome further comprises one or more phospholipids, sphingolipids, bioactive lipids, natural lipids, cholesterol, sterols or a combination thereof.

56. (canceled)

57. The liposome of claim 55, wherein the one or more phosphoplipids is selected from the group consisting of: hydrogenated soy phosphotidylcholine (HSPC); 1,2-didecanoyl-sn-glycero-3-phosphocholine (DDPC); 1,2-dierucoyl-sn-glycero-3-phosphate (DEPA); 1,2-dielaidoyl-sn-glycero-3-phosphocholine (DEPC); 1,2-dierucoyl-sn-glycero-3-phosphoethanolamine (DEPE); 1, 2-dielaidoyl-phosphatidylglycerol (DEPG); 1,2-dilinoleoyl-sn-glycero-3-phosphocholine (DLOPC); 1,2-dilauroyl-sn-glycero-3-phosphate (DLPA); 1,2-dilauroyl-sn-glycero-3-phosphocholine (DLPC); 1,2-dilauroyl-sn-glycero-3-phosphoethanolamine (DLPE); 1,2-dilauroyl-sn-glycero-3-phosphatidylglycerol (DLPG); 1,2-dilauroyl-sn-glycero-3-phosphoserine (DLPS); 1,2-dimyristoyl-sn-glycero-3-phosphate (DMPA); 1,2-dimyristoyl-sn-glycero-3-phosphocholine (DMPC); 1,2-dimyristoyl-sn-glycero-3-phosphoethanolamine (DMPE); 1,2-dimyristoyl-sn-glycero-3-phosphatidylglycerol (DMPG); 1,2-dimyristoyl-sn-glycero-3-phosphoserine (DMPS); 1,2-dioleoyl-sn-glycero-3-phosphate (DOPA); 1,2-dioleoyl-sn-glycero-3-phosphocholine (DOPC); 1,2-dioleoyl-sn-glycero-3-phosphoethanolamine (DOPE); 1,2-dioleoyl-sn-glycero-3-phosphatidylglycerol (DOPG); 1,2-dioleoyl-sn-glycero-3-phosphoserine (DOPS); 1,2-dipalmitoyl-sn-glycero-3-phosphate (DPPA); 1,2-dipalmitoyl-sn-glycero-3-phosphocholine (DPPC); 1,2-dipalmitoyl-sn-glycero-3-phosphoethanolamine (DPPE); 1,2-dipalmitoyl-sn-glycero-3-phosphatidylglycerol (DPPG); 1,2-dipalmitoyl-sn-glycero-3-phosphoserine (DPPS); 1,2-distearoyl-sn-glycero-3-phosphate (DSPA); 1,2-distearoyl-sn-glycero-3-phosphocholine (DSPC); 1,2-distearoyl-sn-glycero-3-phosphoethanolamine (DSPE); 1,2-distearoyl-sn-glycero-3-phosphatidylglycerol (DSPG); 1,2-distearoyl-sn-glycero-3-phosphoserine (DSPS); 1-myristoyl-2-palmitoyl-sn-glycero 3-phosphocholine (MPPC); 1-myristoyl-2-stearoyl-sn-glycero-3-phosphocholine (MSPC); 1-palmitoyl-2-myristoyl-sn-glycero-3-phosphocholine (PMPC); 1-palmitoyl-2-oleoyl-sn-glycero-3-phosphocholine (POPC); 1-palmitoyl-2-oleoyl-sn-glycero-3-phosphoethanolamine (POPE); 1-palmitoyl-2-oleoyl-sn-glycero-3-phosphatidylglycerol (POPG); 1-palmitoyl-2-stearoyl-sn-glycero-3-phosphocholine (PSPC); 1-stearoyl-2-myristoyl-sn-glycero-3-phosphocholine (SMPC); and 1-stearoyl-2-oleoyl-sn-glycero-3-phosphocholine (SOPC); 1-stearoyl-2-palmitoyl-sn-glycero-3-phosphocholine (SPPC); and a combination thereof.

58.-63. (canceled)

64. The liposome of claim 1, wherein the liposome further comprises a fluorophore, a contrast agent, or a combination thereof.

65. A pharmaceutical composition comprising a liposome of claim 1, and a pharmaceutically acceptable excipient.

66. A method of treating a cancer in a patient, the method comprising:
(a) administering to the patient a therapeutically effective amount of a liposome of claim 2; and
(b) irradiating the patient to break the liposome.

67. A method of imaging a cancer in a patient, the method comprising:
(a) administering to the patient a therapeutically effective amount of a liposome of claim 1;
(b) irradiating the patient to break the liposome; and
(c) imaging the patient.

68. The liposome of claim 2, wherein the conjugate comprising a lysophospholipid and a photosensitizer is present at about 0.01 mol percent to about 1 mole percent in the liposome.

69. The liposome of claim 2, wherein the first phospholipid is selected from the group consisting of: hydrogenated soy phosphotidylcholine (HSPC); 1,2-didecanoyl-sn-glycero-3-phosphocholine (DDPC); 1,2-dierucoyl-sn-glycero-3-phosphate (DEPA); 1,2-dielaidoyl-sn-glycero-3-phosphocholine (DEPC); 1,2-dierucoyl-sn-glycero-3-phosphoethanolamine (DEPE); 1, 2-dielaidoyl-phosphatidylglycerol (DEPG); 1,2-dilinoleoyl-sn-glycero-3-phosphocholine (DLOPC); 1,2-dilauroyl-sn-glycero-3-phosphate (DLPA); 1,2-dilauroyl-sn-glycero-3-phosphocholine (DLPC); 1,2-dilauroyl-sn-glycero-3-phosphoethanolamine (DLPE); 1,2-dilauroyl-sn-glycero-3-phosphatidylglycerol (DLPG); 1,2-dilauroyl-sn-glycero-3-phosphoserine (DLPS); 1,2-dimyristoyl-sn-glycero-3-phosphate (DMPA); 1,2-dimyristoyl-sn-glycero-3-phosphocholine (DMPC); 1,2-dimyristoyl-sn-glycero-3-phosphoethanolamine (DMPE); 1,2-dimyristoyl-sn-glycero-3-phosphatidylglycerol (DMPG); 1,2-dimyristoyl-sn-glycero-3-phosphoserine (DMPS); 1,2-dioleoyl-sn-glycero-3-phosphate (DOPA); 1,2-dioleoyl-sn-glycero-3-phosphocholine (DOPC); 1,2-dioleoyl-sn-glycero-3-phosphoethanolamine (DOPE); 1,2-dioleoyl-sn-glycero-3-phosphatidylglycerol (DOPG); 1,2-dioleoyl-sn-glycero-3-phosphoserine (DOPS); 1,2-dipalmitoyl-sn-glycero-3-phosphate (DPPA); 1,2-dipalmitoyl-sn-glycero-3-phosphocholine (DPPC); 1,2-dipalmitoyl-sn-glycero-3-phosphoethanolamine (DPPE); 1,2-dipalmitoyl-sn-glycero-3-phosphatidylglycerol (DPPG); 1,2-dipalmitoyl-sn-glycero-3-phosphoserine (DPPS); 1,2-distearoyl-sn-glycero-3-phosphate (DSPA); 1,2-distearoyl-sn-glycero-3-phosphocholine (DSPC); 1,2-distearoyl-sn-glycero-3-phosphoethanolamine (DSPE); 1,2-distearoyl-sn-glycero-3-phosphatidylglycerol (DSPG); 1,2-distearoyl-sn-glycero-3-phosphoserine (DSPS); 1-myristoyl-2-palmitoyl-sn-glycero 3-phosphocholine (MPPC); 1-myristoyl-2-stearoyl-sn-glycero-3-phosphocholine (MSPC); 1-palmitoyl-2-myristoyl-sn-glycero-3-phosphocholine (PMPC); 1-palmitoyl-2-oleoyl-sn-glycero-3-phosphocholine (POPC); 1-palmitoyl-2-oleoyl-sn-glycero-3-phosphoethanolamine (POPE); 1-palmitoyl-2-oleoyl-sn-glycero-3-phosphatidylglycerol (POPG); 1-palmitoyl-2-stearoyl-sn-glycero-3-phosphocholine (PSPC); 1-stearoyl-2-myristoyl-sn-glycero-3-phosphocholine (SMPC); and 1-stearoyl-2-oleoyl-sn-glycero-3-phosphocholine (SOPC); 1-stearoyl-2-palmitoyl-sn-glycero-3-phosphocholine (SPPC), and combinations thereof; and
wherein the strained cyclooctyne comprises an aza-dibenzocyclooctyne (ADIBO), dibenzocyclooctyne (DIBO), (OCT), aryl-less octyne (ALO), monofluorinated cyclooctyne (MOFO), difluorinated cyclooctyne (DIFO), biarylazacyclooctynone (BARAC), or a dimethoxyazacyclooctyne (DIMAC) moiety.

70. The liposome of claim 69, wherein the first derivatized phospholipid further comprises a linker.

71. The liposome of claim 69, wherein the linker is a polyethylene glycol having a molecular weight selected from the group consisting of 350 g/mol, 550 g/mol, 750 g/mol, 1000 g/mol, 2000 g/mol, 3000 g/mol, 5000 g/mol, 10,000 g/mol, 20,000 g/mol and 30,000 g/mol; and
wherein the first derivatized phospholipid comprises DSPE-PEG₂₀₀₀.

72. The liposome of claim 2, wherein the second phospholipid is selected from the group consisting of: hydrogenated soy phosphotidylcholine (HSPC); 1,2-didecanoyl-sn-glycero-3-phosphocholine (DDPC); 1,2-dierucoyl-sn-glycero-3-phosphate (DEPA); 1,2-dielaidoyl-sn-glycero-3-phosphocholine (DEPC); 1,2-dierucoyl-sn-glycero-3-phosphoethanolamine (DEPE); 1, 2-dielaidoyl-phosphatidylglycerol (DEPG); 1,2-dilinoleoyl-sn-glycero-3-phosphocholine (DLOPC); 1,2-dilauroyl-sn-glycero-3-phosphate (DLPA); 1,2-dilauroyl-sn-glycero-3-phosphocholine (DLPC); 1,2-dilauroyl-sn-glycero-3-phosphoethanolamine (DLPE); 1,2-dilauroyl-sn-glycero-3-phosphatidylglycerol (DLPG); 1,2-dilauroyl-sn-glycero-3-phosphoserine (DLPS); 1,2-dimyristoyl-sn-glycero-3-phosphate (DMPA); 1,2-dimyristoyl-sn-glycero-3-phosphocholine (DMPC); 1,2-dimyristoyl-sn-glycero-3-phosphoethanolamine (DMPE); 1,2-dimyristoyl-sn-glycero-3-phosphatidylglycerol (DMPG); 1,2-dimyristoyl-sn-glycero-3-phosphoserine (DMPS); 1,2-dioleoyl-sn-glycero-3-phosphate (DOPA); 1,2-dioleoyl-sn-glycero-3-phosphocholine (DOPC); 1,2-dioleoyl-sn-glycero-3-phosphoethanolamine (DOPE); 1,2-dioleoyl-sn-glycero-3-phosphatidylglycerol (DOPG); 1,2-dioleoyl-sn-glycero-3-phosphoserine (DOPS); 1,2-dipalmitoyl-sn-glycero-3-phosphate (DPPA); 1,2-dipalmitoyl-sn-glycero-3-phosphocholine (DPPC); 1,2-dipalmitoyl-sn-glycero-3-phosphoethanolamine (DPPE); 1,2-dipalmitoyl-sn-glycero-3-phosphatidylglycerol (DPPG); 1,2-dipalmitoyl-sn-glycero-3-phosphoserine (DPPS); 1,2-distearoyl-sn-glycero-3-phosphate (DSPA); 1,2-distearoyl-sn-glycero-3-phosphocholine (DSPC); 1,2-distearoyl-sn-glycero-3-phosphoethanolamine (DSPE); 1,2-distearoyl-sn-glycero-3-phosphatidylglycerol (DSPG); 1,2-distearoyl-sn-glycero-3-phosphoserine (DSPS); 1-myristoyl-2-palmitoyl-sn-glycero 3-phosphocholine (MPPC); 1-myristoyl-2-stearoyl-sn-glycero-3-phosphocholine (MSPC); 1-palmitoyl-2-myristoyl-sn-glycero-3-phosphocholine (PMPC); 1-palmitoyl-2-oleoyl-sn-glycero-3-phosphocholine (POPC); 1-palmitoyl-2-oleoyl-sn-glycero-3-phosphoethanolamine (POPE); 1-palmitoyl-2-oleoyl-sn-glycero-3-phosphatidylglycerol (POPG); 1-palmitoyl-2-stearoyl-sn-glycero-3-phosphocholine (PSPC); 1-stearoyl-2-myristoyl-sn-glycero-3-phosphocholine (SMPC); and 1-stearoyl-2-oleoyl-sn-glycero-3-phosphocholine (SOPC); 1-stearoyl-2-palmitoyl-sn-glycero-3-phosphocholine (SPPC); and combinations thereof.

73. The liposome of claim 2, wherein the polyethylene glycol polymer is terminated with an alkoxyl, carboxyl, amine, biotin, maleimide, succinyl, N-hydroxysuccinimidyl ester, sulfo-N-hydroxysuccinimidyl ester, silane, pyridyldithiol or cyanur moiety.

74. The liposome of claim 2, wherein the liposome comprises an anionic lipid.

75. The liposome of claim 74, wherein the anionic lipid is selected from the group consisting of: 1,2-dioleoyl-sn-glycero-3-phospho-(1′-rac-glycerol) (DOPG); phosphatidyl glycerol (PG); 1,2-dimyristoyl-sn-glycero-3-phospho-(1′-rac-glycerol) (DMPG); phosphatidylserine (PS); 1,2-dioleoyl-sn-glycero-3-phospho-L-serine (DOPS); and dicetylphosphate (DCP).

76. The liposome of claim 2, wherein the liposome comprises a cationic lipid.

77. The liposome of claim 2, wherein the liposome further comprises a cargo selected from the group consisting of one or more therapeutic compounds; one or more polymeric nanoparticles; one or more protein-based nanoparticles; one or more dendrimeric structures; one or more chitosan nanoparticle; one or more polyglycan structures; one or more inorganic nanoparticles; one or more imaging agents; one or more kinase inhibitors; one or more biologics; and combinations of two or more thereof;
wherein the one or more kinase inhibitors is a receptor tyrosine kinase inhibitor.

78. The liposome of claim 77, wherein one or more of the one or more therapeutic compounds exhibit synergy with the photosensitizer.

79. The liposome of claim 2, wherein the liposome further comprises one or more phospholipids, sphingolipids, bioactive lipids, natural lipids, cholesterol, sterols or a combination thereof.

80. The liposome of claim 79, wherein the one or more phosphoplipids is selected from the group consisting of: hydrogenated soy phosphotidylcholine (HSPC); 1,2-didecanoyl-sn-glycero-3-phosphocholine (DDPC); 1,2-dierucoyl-sn-glycero-3-phosphate (DEPA); 1,2-dielaidoyl-sn-glycero-3-phosphocholine (DEPC); 1,2-dierucoyl-sn-glycero-3-phosphoethanolamine (DEPE); 1, 2-dielaidoyl-phosphatidylglycerol (DEPG); 1,2-dilinoleoyl-sn-glycero-3-phosphocholine (DLOPC); 1,2-dilauroyl-sn-glycero-3-phosphate (DLPA); 1,2-dilauroyl-sn-glycero-3-phosphocholine (DLPC); 1,2-dilauroyl-sn-glycero-3-phosphoethanolamine (DLPE); 1,2-dilauroyl-sn-glycero-3-phosphatidylglycerol (DLPG); 1,2-dilauroyl-sn-glycero-3-phosphoserine (DLPS); 1,2-dimyristoyl-sn-glycero-3-phosphate (DMPA); 1,2-dimyristoyl-sn-glycero-3-phosphocholine (DMPC); 1,2-dimyristoyl-sn-glycero-3-phosphoethanolamine (DMPE); 1,2-dimyristoyl-sn-glycero-3-phosphatidylglycerol (DMPG); 1,2-dimyristoyl-sn-glycero-3-phosphoserine (DMPS); 1,2-dioleoyl-sn-glycero-3-phosphate (DOPA); 1,2-dioleoyl-sn-glycero-3-phosphocholine (DOPC); 1,2-dioleoyl-sn-glycero-3-phosphoethanolamine (DOPE); 1,2-dioleoyl-sn-glycero-3-phosphatidylglycerol (DOPG); 1,2-dioleoyl-sn-glycero-3-phosphoserine (DOPS); 1,2-dipalmitoyl-sn-glycero-3-phosphate (DPPA); 1,2-dipalmitoyl-sn-glycero-3-phosphocholine (DPPC); 1,2-dipalmitoyl-sn-glycero-3-phosphoethanolamine (DPPE); 1,2-dipalmitoyl-sn-glycero-3-phosphatidylglycerol (DPPG); 1,2-dipalmitoyl-sn-glycero-3-phosphoserine (DPPS); 1,2-distearoyl-sn-glycero-3-phosphate (DSPA); 1,2-distearoyl-sn-glycero-3-phosphocholine (DSPC); 1,2-distearoyl-sn-glycero-3-phosphoethanolamine (DSPE); 1,2-distearoyl-sn-glycero-3-phosphatidylglycerol (DSPG); 1,2-distearoyl-sn-glycero-3-phosphoserine (DSPS); 1-myristoyl-2-palmitoyl-sn-glycero 3-phosphocholine (MPPC); 1-myristoyl-2-stearoyl-sn-glycero-3-phosphocholine (MSPC); 1-palmitoyl-2-myristoyl-sn-glycero-3-phosphocholine (PMPC); 1-palmitoyl-2-oleoyl-sn-glycero-3-phosphocholine (POPC); 1-palmitoyl-2-oleoyl-sn-glycero-3-phosphoethanolamine (POPE); 1-palmitoyl-2-oleoyl-sn-glycero-3-phosphatidylglycerol (POPG); 1-palmitoyl-2-stearoyl-sn-glycero-3-phosphocholine (PSPC); 1-stearoyl-2-myristoyl-sn-glycero-3-phosphocholine (SMPC); and 1-stearoyl-2-oleoyl-sn-glycero-3-phosphocholine (SOPC); 1-stearoyl-2-palmitoyl-sn-glycero-3-phosphocholine (SPPC); and a combination thereof.

81. The liposome of claim 2, wherein the liposome further comprises a fluorophore, a contrast agent, or a combination thereof.

82. A pharmaceutical composition comprising a liposome of claim 2, and a pharmaceutically acceptable excipient.

83. A method of imaging a cancer in a patient, the method comprising:
(a) administering to the patient a therapeutically effective amount of a liposome of claim 2;
(b) irradiating the patient to break the liposome; and
(c) imaging the patient.
